(19) United States
(12) Patent Application Publication
(10) Pub. No.: US 2026/0034259 A1
(43) Pub. Date: Feb. 5, 2026

(54) TRANSFER DEVICE

(71) Applicant: KyooBe Tech GmbH, Leinfelden-Echterdingen (DE)

(72) Inventors: David WELLER, Esslingen am Neckar (DE); Vincent EUCHNER, Stuttgart (DE); Lena SCHOBER, Grossbottwar (DE)

(73) Assignee: KyooBe Tech GmbH, Leinfelden-Echterdingen (DE)

(21) Appl. No.: 19/351,822

(22) Filed: Oct. 7, 2025

Related U.S. Application Data
(63) Continuation of application No. PCT/EP2024/062205, filed on May 3, 2024.

(30) Foreign Application Priority Data
May 5, 2023 (DE) 10 2023 111 765.3

(51) Int. Cl. A61L 2/26; A61L 2/00; A61L 2/07; A61L 2/24; B01L 3/00; B25J 21/00

(52) U.S. Cl. CPC A61L 2/26; A61L 2/0023; A61L 2/07; A61L 2/24; B01L 3/563; B25J 21/005; A61L 2202/121; A61L 2202/14; A61L 2202/21; B01L 2200/026

(57) ABSTRACT

A transfer device for enabling an aseptic transition between a first object, in particular a first process unit, and a second object, in particular a second process unit. Furthermore, a system consisting of at least two objects, in particular process units, and to a system consisting of three or four or more objects and/or process units.

### RELATED APPLICATION

This application is a continuation of international application No. PCT/EP2024/062205 filed on May 3, 2024, and claims the benefit of German application No. 10 2023 111 765.3 filed on May 5, 2023, which are incorporated herein by reference in their entirety and for all purposes.

### FIELD OF DISCLOSURE

The present invention relates to a transfer device for enabling an in particular aseptic transition between a first object, in particular a first process unit, and a second object, in particular a second process unit.

### BACKGROUND

In particular in the pharmaceutical field, for example for personalized therapeutics, a plurality of process units and/or process rooms and/or process areas are used for manufacturing a biological-pharmaceutical product, for example, in order to be able to carry out different treatment processes for the production of the product. Often it can be necessary to connect such objects, for example the process units and/or process rooms and/or process areas, in order to be able to perform a specific treatment procedure. For example, it can be necessary to efficiently channel a material and/or an object from one process unit into and/or out of another process unit. This requires the highest degree of sterility.

Furthermore, these highly personalized therapeutics are associated with high costs, in particular personnel costs, due to their individual and/or very small production runs, and there is often no possibility for large-scale application and/or industrial production of individual and/or very small batches.

### SUMMARY OF THE INVENTION

The present invention is based on the object of providing a transfer device for enabling an in particular aseptic transition between a first object, in particular a first process unit, and a second object, in particular a second process unit, which is preferably able to be operated in an automated manner.

This object is achieved according to the invention by the features of the independent claim. Advantageous refinements of the invention are described in the dependent claims.

A transfer device according to the invention for enabling an in particular aseptic transition between a first object, in particular a first process unit, and a second object, in particular a second process unit, is able to be at least partially installed, in particular disposed and/or assembled, in the first object, in particular the first process unit, and is able to be at least partially installed, in particular disposed and/or assembled, in the second object, in particular the second process unit.

For example, a first part of the transfer device can be able to be installed in the first object, in particular the first process unit, and a second part of the transfer device can be able to be installed in the second object, in particular the second process unit.

The transfer device according to the invention has the following:
a first door element, which in the installed state is disposed on the first object, in particular the first process unit, for opening and closing an access opening formed in a wall portion of the first object, in particular of the first process unit, and leading to an interior of the first object, in particular of the first process unit;
a second door element, which in the installed state is disposed on the second object, in particular the second process unit, for opening and closing an access opening formed in a wall portion of the second object, in particular of the second process unit, and leading to an interior of the second object, in particular of the second process unit;
a locking apparatus for locking the first door element in a closed state and for releasing the first door element for opening the latter, wherein the locking apparatus in the installed state is disposed in and/or on the first object, in particular the first process unit; and
an opener apparatus for opening and closing at least one of the door elements, wherein the opener apparatus in the installed state is disposed in and/or on the second object, in particular the second process unit,
   wherein the first door element and the second door element are able to be brought to engage with one another and in the engaged state, when the first door element is released by means of the locking apparatus, are able to be conjointly removed from the access openings by means of the opener apparatus, so as to enable the in particular aseptic transition between the first object, in particular the first process unit, and the second object, in particular the second process unit.

The transfer device according to the invention can be particularly advantageously able to be installed and used in process units configured as described herein. However, it is understood that the transfer device according to the invention is not limited to such process units. Therefore, a first and/or a second object may also be, for example, any solid volumetric body, for example a vessel and/or a container and/or a production room and/or a conveyor, and/or even only a part of such a volumetric body, for example a wall or a wall structure.

Advantageously, it can be provided that the first object is a process unit, in particular a first process unit, and that the second object is a further process unit, in particular a second process unit.

It can be provided, for example, that a process unit is designed as a treatment box, e.g. a patient box, in particular for receiving and/or for transporting and/or treating a treatment object.

Additionally or alternatively, it can be provided that a process unit is designed as a tool box, in particular for treating a treatment object, which in particular can comprise a tool unit in particular for treating the treatment object and/or a sensor unit, in particular for analyzing the treatment object.

Additionally or alternatively, it can be provided that a process unit is designed as a supply box in particular for providing a material, e.g. a consumable material, and/or a substance, which can be required for treating a treatment object.

It can be provided, for example, that the first process unit or the second process unit is designed as a treatment box and the respective other process unit is designed as a tool box and/or a supply box.

Additionally or alternatively, it can be provided, for example, that the first process unit or the second process unit is designed as a tool box and the respective other process unit is designed as a supply box.

It can be provided, for example, that the in particular aseptic transition between the objects, in particular process units, serves to supply any item, for example a treatment object, a tool unit, a sensor unit, a material and/or a substance, from one process unit to the other process unit, in particular temporarily and/or permanently.

The in particular aseptic transition between the objects, in particular process units, can be used bidirectionally and/or unidirectionally, for example for an exchange of any item between the objects, in particular process units. In other words, the in particular aseptic transition between the objects, in particular process units, can be a bidirectional and/or a unidirectional transition.

For example, a treatment object can include at least one of the following:
an educt, in particular a cell-containing material, for example a cell-containing primary material, and/or a biological-pharmaceutical material and/or a biological-pharmaceutical workpiece,
an intermediate product, in particular a cell-containing intermediate product and/or a biological-pharmaceutical intermediate product,
a product, for example the product to be manufactured, in particular a biological-pharmaceutical product, for example the biological-pharmaceutical product to be manufactured.

It is to be understood that the previous enumeration is exemplary and not exhaustive. Alternatively or additionally, it can be provided that the treatment object comprises one or a plurality of, and/or a combination of, the enumeration elements described above.

It can be provided that a process unit is movable and/or transportable and/or in particular automatically displaceable relative to its environment.

For example, a process unit can comprise an in particular freely moving transport vehicle or be designed as such, and/or be able to be coupled to an in particular freely moving transport vehicle.

It can be provided that the in particular freely moving transport vehicle is guided by induction loops and/or is magnetically guided, in particular guided by permanent magnets and/or solenoids. Additionally or alternatively, a sensor-based guidance, in particular a camera-based guidance and/or a radar/LIDAR-based guidance, can be provided.

It can be provided that a process unit has one or a plurality of coupling devices by means of which the process unit is connectable, in particular mechanically able to be coupled, to one or a plurality of further process units. For example, the coupling devices can be fully mechanically operable. In other words, the connectable process units can be connectable to one another fully automatically.

It can be provided, for example, that a coupling device has one or a plurality of locking elements for locking and/or interlocking the process unit to/with at least one further process unit, and/or is designed as such locking element(s).

For example, it can be provided that the at least one further process unit can have coupling devices, in particular locking elements, corresponding to the locking elements.

It can be conceivable, for example, that such a locking element can be designed as a holding element for engaging in a holding contour, and/or as a holding contour for receiving a holding element. The holding elements can, for example, be motorized in such a way that locking and/or interlocking can preferably take place in an automated manner, in particular in a fully automatic manner.

Additionally or alternatively, it can be provided, for example, that a coupling device has one or a plurality of positioning elements for positioning the process unit with and/or relative to at least one further process unit, and/or is designed as such positioning element(s).

For example, it can be provided that the at least one further process unit can have coupling devices, in particular positioning elements, corresponding to the positioning elements.

It can be conceivable, for example, that such a positioning element can be designed as a positioning protrusion for insertion into a positioning receptacle and/or as a positioning receptacle for receiving a positioning protrusion.

Additionally or alternatively, it can be conceivable that such a positioning element and corresponding positioning element are able to be brought to magnetically engage with one another. For example, positioning elements of this type can additionally be part of an apparatus, in particular a coil apparatus, for the wireless and/or inductive transmission of energy between coupled process units.

Additionally or alternatively, it can be provided, for example, that a coupling device has one or a plurality of plug elements and/or socket elements for the transmission of energy and/or fluids, in particular gases, gas mixtures and/or liquids, between coupled process units, and/or are designed as such plug elements and/or socket elements.

For example, it can be provided that the at least one further process unit can have coupling devices, in particular plug elements and/or socket elements, corresponding to the plug elements and/or socket elements.

Additionally or alternatively, it can be provided, for example, that a coupling device has an apparatus, in particular a coil apparatus, for the wireless and/or inductive transmission of energy between coupled process units, and/or is designed as such an apparatus.

For example, it can be provided that the at least one further process unit can have at least one coupling device, in particular complementary coil apparatus, corresponding to this apparatus, in particular coil apparatus.

It can be provided that the wireless and/or inductive transmission of energy between coupled process units can take place unidirectionally and/or bidirectionally.

It can be provided that a process unit, in particular a treatment box and/or a tool box and/or a supply box, defines a process cell, in particular an aseptic process cell. In other words, a process unit, in particular a process unit designed as a treatment box and/or tool box and/or supply box, can be a process cell, in particular an aseptic process cell.

It can be provided that each process unit, in particular treatment box and/or tool box and/or supply box, has an in particular lockable interior space.

Said interior space can correspond to the respective interior of the first and/or the second process unit, and/or be and/or form this interior partially or completely.

It can be provided that each process unit comprises a shell wall which encloses the respective interior space and/or the respective interior, thus defining the latter.

Said shell wall can comprise the respective wall portion of the first and/or of the second process unit, and/or be partially formed by the respective wall portion.

An in particular aseptic production area can be able to be defined, or be defined and/or formed by the interior space and/or the interior of a process unit. In other words, it can be provided that the interior space and/or the interior of a process unit is an in particular aseptic production area.

In other words, the respective access opening of the first and of the second process unit can enable a respective access to a respective in particular aseptic production area of the first and of the second process unit.

In other words, it can be provided that the first door element which in the installed state is disposed on the first process unit serves to open and close the access opening to an in particular aseptic production area in the interior of the first process unit.

In other words, it can be provided that the second door element which in the installed state is disposed on the second process unit serves to open and close the access opening to an in particular aseptic production area in the interior of the second process unit.

It can be provided that the first door element comprises an in particular truncated conical or truncated pyramidal portion which tapers in a direction which in the closed state of the first door element points toward the interior of the first object, in particular of the first process unit.

It is understood that a truncated pyramidal portion can have a triangular, quadrangular, or polygonal cross section.

It can be provided that a circumferential surface of said portion of the first door element, in the closed state of the first door element, rests on an inner circumferential surface of the access opening of the first object, in particular of the first process unit.

Additionally or alternatively, it can be provided that the second door element comprises an in particular truncated conical or truncated pyramidal portion which tapers in a direction which in the closed state of the second door element is directed outward relative to the second object, in particular the second process unit.

It can be provided that a circumferential surface of said portion of the second door element in the closed state of the second door element rests on an inner circumferential surface of the access opening of the second object, in particular of the second process unit.

It can be provided that the transfer device has a sealing apparatus which is formed from, or comprises, a plurality of sealing sub-elements.

It can be provided that in the closed state of the first door element, the first door element and an inner circumferential surface of the access opening of the first object, in particular of the first process unit, are able to be sealed, or are sealed, in relation to the outside by means of the sealing apparatus.

Additionally or alternatively, it can be provided that in the closed state of the second door element, the second door element and an inner circumferential surface of the access opening of the second object, in particular of the second process unit, are able to be sealed, or are sealed, in relation to the outside by means of the sealing apparatus.

Additionally or alternatively, it can be provided that in the engaged state of the first and the second door element, respective surfaces of both door elements, which in the respective closed state of the corresponding door elements are directed outward relative to the corresponding object, in particular the corresponding process unit, are able to be sealed, or are sealed, in relation to the outside by means of the sealing apparatus.

Additionally or alternatively, it can be provided that in a state in which the respective wall portions of the objects, in particular of the process units, rest on one another in order to bring the first and the second door element to engage, respective opening regions of both access openings of both objects, in particular process units, are able to be sealed, or are sealed, in relation to the outside by means of the sealing apparatus.

It can be provided that the sealing apparatus, in particular each sealing sub-element, has a closed and in particular circular or polygonal, e.g. quadrangular and/or quadrangular-like, contour.

It can be provided, for example, that such a polygonal, for example quadrangular and/or quadrangular-like, contour can have rounded corner regions, and/or radiused corner regions, preferably so as to guarantee a hygienically compliant design embodiment, in particular a hygienic design.

It can be provided that the sealing apparatus comprises a plurality of, for example four, sealing sub-elements.

For example, a first sealing sub-element can be disposed on a peripheral region of the first door element, which in the closed state of the first door element is adjacent to the inner circumferential surface of the access opening of the first object, in particular of the first process unit, and is directed outward relative to the first object, in particular the first process unit.

For example, a second sealing sub-element can be able to be disposed, or can be disposed, on an edge region of the access opening of the first object, in particular of the first process unit, which in the closed state of the first door element is adjacent to the first door element, in particular to the said peripheral region of the first door element, and is directed outward relative to the first object, in particular the first process unit.

For example, a third sealing sub-element can be disposed on a peripheral region of the second door element, which in the closed state of the second door element is adjacent to the inner circumferential surface of the access opening of the second object, in particular of the second process unit, and is directed outward relative to the second object, in particular the second process unit.

For example, a fourth sealing sub-element can be able to be disposed, or can be disposed, on an edge region of the access opening of the second object, in particular of the second process unit, which in the closed state of the second door element is adjacent to the second door element, in particular to the said peripheral region of the second door element, and is directed outward relative to the second object, in particular the second process unit.

It can be provided that the first and the third sealing sub-element are disposed directly next to one another, in particular in the engaged state of the first and the second door element.

The respective surfaces of both door elements which in the respective closed state of the corresponding door elements are directed outward relative to the corresponding object, in particular the corresponding process unit, and in particular are bordered by the respectively assigned first and third sealing sub-element, can be able to be sealed, or can be sealed, in relation to the outside by means of the first and the third sealing sub-element. This can be understood to mean in particular that a respective surface of the mutually engaged door elements, which face one another in the engaged state of the door elements and/or on which the door elements rest on one another, can be able to be sealed, or can be sealed, in relation to the outside by means of the first and the third sealing sub-element.

It can be provided that the second and the fourth sealing sub-element can be able to be disposed, or can be disposed, directly next to one another, in particular in a, or the, state in which the respective wall portions of the objects, in particular of the process units, rest on one another for a, or the, bringing of the first and the second door element to engage with one another.

Respective opening regions of both access openings of both objects, in particular process units, can be able to be sealed, or can be sealed, in relation to the outside by means of the second and the fourth sealing sub-element.

It can be provided that a respective sealing sub-element has a circular cross section. Additionally or alternatively, it can be provided that a respective sealing sub-element has a polygonal, e.g. wedge-shaped, cross section.

For example, a respective sealing sub-element can have at least one, for example conically, tapered portion in the cross section. The tapered portion can taper toward the outside relative to the respective object, in particular the respective process unit, and in particular the respective door element, on which the respective sealing sub-element can be disposed. As a result, a sealing surface can advantageously be able to be reduced, whereby high contact forces which can be required for large sealing surfaces can be avoidable.

It can be provided, for example, that a respective sealing sub-element is able to be manufactured, or is manufactured, from plastics material. For example, a respective sealing sub-element can comprise and/or at least be partially formed from polytetrafluoroethylene (PTFE) and/or polyvinyl chloride (PVC) and/or silicone. This can advantageously enable a positive autoclaving capability.

It can be conceivable, for example, that the sealing apparatus, for example, comprises two sealing sub-elements, preferably the second and the third sealing sub-element, or the first and the fourth sealing sub-element.

For example, in particular in the engaged state of the first and the second door element, the respective surfaces of both door elements, which in the respective closed state of the corresponding door elements are directed outward relative to the corresponding object, in particular the corresponding process unit, can be able to be sealed, or can be sealed, by means of the third and/or the first sealing sub-element.

For example, respective opening regions of both access openings of both objects, in particular process units, can be able to be sealed, or can be sealed, in relation to the outside by means of the second and/or the fourth sealing sub-element, in particular when the objects, in particular process units, are disposed on one another and/or are coupled to one another.

It can be provided that the second door element comprises a coupling element which is provided in and/or on the second door element and forms at least a part of the second door element.

The first door element can be able to be brought to engage with the second door element and able to be held thereon by means of the coupling element.

It can be provided that the coupling element is configured to bring the first door element to engage with the second door element and to be held thereon in particular in a magnet-based, force-fitting, form-fitting and/or vacuum-based manner.

For example, it can be provided that the coupling element forms a part of the second door element which is directed outward relative to the second object, in particular the second process unit, and in particular has a flush transition to the remaining part of the second door element, which part is directed outward relative to the second object, in particular the second process unit.

It can be provided, for example, that the coupling element is designed as at least one solenoid, or comprises at least one solenoid, and that the first door element is designed partially or completely as a magnetic counter-element, or comprises such a magnetic counter-element.

For example, the coupling element can comprise one or a plurality of, e.g. two, solenoids.

It can be provided that the coupling element designed as a solenoid is actuatable by means of a control unit of the transfer device and/or by means of a control unit assignable or assigned to the transfer device, in particular in order to bring the first door element to engage with the second door element and to hold it thereon.

It can be provided that the coupling element is provided, in particular centrically, in and/or on the in particular truncated conical or truncated pyramidal portion of the second door element.

It can be provided, for example, that the coupling element is formed as and/or comprises a bayonet apparatus. Male and/or female bayonet elements can be provided in and/or on the second door element. It is understood that female and/or male bayonet counter-elements can be correspondingly provided in and/or on the first door element in order to be able to be brought to engage with the bayonet elements. Furthermore, a rotary apparatus can be provided in and/or on the first and/or the second door element, so as to be able to bring the bayonet elements and/or bayonet counter-elements to engage, in particular in a rotational manner. It can be provided, for example, that the rotary apparatus is actuatable by means of a control unit of the transfer device and/or by means of a control unit assignable or assigned to the transfer device, in particular in order to bring the first door element to engage with the second door element and to hold it thereon.

It can be provided, for example, that the coupling element is designed and/or comprises a screw element. The screw element can be provided in and/or on the second door element. It is understood that a screw counter-element can be correspondingly provided in and/or on the first door element, in order to be able to be brought to engage with the screw element. Furthermore, a rotary apparatus can be provided in and/or on the first and/or the second door element in order to be able to bring the screw element and/or the screw counter-element to engage, in particular in a screwing manner. It can be provided, for example, that the rotary apparatus is actuatable by means of a control unit of the transfer device and/or by means of a control unit assignable or assigned to the transfer device, in particular in order to bring the first door element to engage with the second door element and to hold it thereon.

It can be provided, for example, that the coupling element is designed as part of a fluid apparatus for providing a vacuum and/or comprises such a fluid apparatus. The fluid apparatus can be able to be disposed, and/or can be disposed, in and/or on the second object, in particular the second process unit, and can optionally be comprised by the transfer device. The fluid can be a gas or gas mixture, particularly air. By means of the fluid apparatus, a vacuum can be able to be provided between the first and the second door element, in particular by way of the coupling element, when the first and the second door element are disposed so as to rest on one another. As a result, the first and the second door element can be able to be brought to engage with one another and be able to be held on one another, for example by suction. It can be provided, for example, that the fluid apparatus can be actuatable by means of a control unit of the transfer device and/or by means of a control unit assignable or assigned to the transfer device, in particular in order to bring the first door element to engage with the second door element and to hold it thereon.

It can be provided, for example, that the coupling element is formed as and/or comprises an eccentric apparatus. The eccentric apparatus can comprise a shaft on which an eccentric element is formed, and a motion apparatus by means of which the shaft is movable, in particular linearly and/or rotationally movable. The shaft can be provided in and/or on the second door element. The motion apparatus can be able to be disposed, or can be disposed, in and/or on the second object, in particular the second process unit, and can optionally be comprised by the transfer device. A counter-element can be provided in and/or on the first door element in order to be able to be brought to engage with the shaft and in particular with the eccentric element. For example, the shaft and in particular the eccentric element can be able to be positioned linearly relative to the counter-element and subsequently rotatable by means of the motion apparatus, in order to bring the eccentric element and the counter-element to engage with one another and to push the counter-element from the first to the second door element. As a result, the first and the second door element can be able to be brought to engage with one another and to be held on one another. It can be provided, for example, that the motion apparatus is actuatable by means of a control unit of the transfer device and/or by means of a control unit assignable or assigned to the transfer device, in particular in order to bring the first door element to engage with the second door element and to hold it thereon.

It can be provided, for example, that the coupling element is designed as or comprises a wedge apparatus. The wedge apparatus can comprise a displacement element on which a wedge-shaped element is formed, and a motion apparatus by means of which the displacement element is movable, in particular linearly movable. The displacement element can be provided in and/or on the second door element. The motion apparatus can be able to be disposed, or can be disposed, in and/or on the second object, in particular the second process unit, and can optionally be comprised by the transfer device. A wedge-shaped counter-element can be provided in and/or on the first door element, in order to be able to be brought to engage with the displacement element and in particular with the wedge-shaped element. For example, the displacement element, and in particular the wedge-shaped element, can be movable linearly relative to the counter-element by means of the motion apparatus, and subsequently be movable along one another while contacting one another in order to push the counter-element from the first to the second door element. As a result, the first and the second door element can be able to be brought to engage with one another and to be held on one another. It can be provided, for example, that the motion apparatus is actuatable by means of a control unit of the transfer device and/or by means of a control unit assignable or assigned to the transfer device, in particular in order to bring the first door element to engage with the second door element and to hold it thereon.

It is understood that the respective design embodiments of the coupling element can in each case be designed to be encapsulated and/or separate relative to the respective interior of the respective objects, in particular process units.

It can be provided that the first door element and/or the second door element each have an outwardly directed shell surface with a hygienically compliant design embodiment, in particular a hygienic design.

Hygienic design can be understood in particular to mean a design of parts, components and/or production systems that is suitable for cleaning and/or sterilization.

For the hygienically compliant design, the first door element and/or the second door element can have no undercuts on the outside and in particular on the shell surface.

It can be provided that the opener apparatus comprises a pivotable lever element, which, in the installed state in the second object, in particular the second process unit, is pivotably fixed on a first end region of the lever element, and on which the second door element is fastened to a second end region of the lever element.

It can be provided that by means of the lever element, the second door element, or the mutually engaged door elements conjointly, is/are able to be moved, in particular in a pivoting manner, into the interior of the second object, in particular of the second process unit, and is/are thus able to be removed from the access openings.

In other words, it can be provided that the pivotable lever element has the first end region for in particular pivotably fixing in an interior of an, in particular the second, object, preferably of an, in particular the second, process unit, and has the second end region for holding an, in particular the second, door element, in particular extending therebetween by way of a central region.

It can be provided that the pivotable lever element, in particular the second end region of the lever element, is rigidly connected to the second door element, or is movably connected thereto in particular by way of an articulation apparatus.

It can be provided that by means of the lever element, the second door element or the mutually engaged door elements conjointly, are movable in a purely pivoting manner, or in a pivoting and linear manner, into the interior of the second object, in particular of the second process unit, and thus are able to be removed from the access openings.

The lever element can preferably be able to be fixed in the second object, in particular the second process unit, in order to be able to pivot the second door element to a lateral and/or upper surface of the interior of the second object, in particular of the second process unit.

It can be provided that at least a part of the opener apparatus is rotatable and/or pivotable about a center of rotation which is able to be disposed, or is disposed, in the interior of the second object, in particular of the second process unit. For example, the part can be the first and/or the second end region and/or the central region.

It can be provided that the center of rotation is able to be disposed, or is disposed, partially or completely in the interior of the second object, in particular of the second process unit.

It can be provided that the center of rotation is able to be disposed, or is disposed, partially outside the interior of the second object, in particular of the second process unit, e.g. outside and/or in a separate space, in particular interior space, of the second object.

It can be provided, for example, that the center of rotation forms a part of the first end region of the lever element, by way of which the latter is able to be fixed, or is fixed, in the second object, in particular the second process unit, or is formed as a separate component on which the lever element is disposed by way of its first end region and by way of which the lever element is able to be fixed, or is fixed, in the second object, in particular the second process unit.

It can be provided, for example, that the center of rotation is and/or is comprised by part of a drive unit of the opener apparatus.

It can be conceivable here that the lever element is indirectly or directly disposed on and/or fastened to the center of rotation of the drive unit by way of the first end region, preferably so as to be linearly movable, and the lever element is able to be fixed, or is fixed, in the second object, in particular the second process unit, by way of the drive unit and in particular the center of rotation, preferably so as to be able to be movably fixed, or so as to be movably fixed.

It can be provided that the opening and closing of at least one of the door elements is able to be initiated by means of a corresponding rotating and/or pivoting movement. For example, the rotating and/or pivoting movement can be able to take place, or can take place, about the center of rotation.

Preferably, the center of rotation in the installed state, relative to the access opening of the second object, in particular of the second process unit, can be disposed at a spacing which is greater than, or greater than or equal to, a widest opening dimension of the access opening of the second object, in particular of the second process unit, in particular than an opening diameter of the access opening of the second object, in particular of the second process unit.

In particular, said spacing can extend between the center of rotation and a center, in particular a center point, of the access opening of the second object, in particular of the second process unit.

For example, the spacing can be at least 100%, in particular at least 150%, preferably at least 200%, further preferably at least 250%, in particular preferably at least 300%, greater than, or greater than or equal to, the widest opening dimension of the access opening of the second object, in particular of the second process unit, in particular than the opening diameter of the access opening of the second object, in particular of the second process unit.

It can be provided that the opener apparatus comprises a contact pressure apparatus for generating a contact pressure and/or for maintaining tightness of the second door element in relation to the wall portion and/or access opening assigned to the second door element.

The contact pressure apparatus can, for example, be partially fixed on the center of rotation and can be partially fixed or is partially fixed on the second object, in particular the second process unit. In particular, the part proximal to the center of rotation can be disposed so as to be spaced apart from the lever element and/or from the second door element and be rigidly connected to the center of rotation.

It can be provided, for example, that the part proximal to the center of rotation is designed as, and/or comprises, a wedge-like activation protrusion, and the part proximal to the object is designed as, and/or comprises, an active or passive linear actuator with an activation contour. For example, an active linear actuator can be magnetically activatable. A passive linear actuator, for example, can operate on a spring-force basis. By means of the linear actuator, the activation contour can be able to be brought to engage with the wedge-like activation protrusion and/or to be held thereon. In the engaged state, a deployment of the linear actuator can increase a contact pressure and/or a tightness of the second door element in relation to the wall portion and/or access opening assigned to the second door element, and a corresponding retraction of the linear actuator can cause the opposite. During deployment and retraction, the activation contour in the engaged state can be able to be moved, or can be moved, along an oblique surface of the wedge-like activation protrusion. It is understood that here the pivoting movement which is able to be generated by means of the activation contour and the activation protrusion is transferred by way of the center of rotation to the second door element.

It can be provided, for example, that the part proximal to the center of rotation is formed as and/or comprises a wedge-like activation protrusion, and the part proximal to the object is formed as and/or comprises an activation contour preloaded with a spring element. The activation contour can press onto the activation protrusion, in particular an oblique surface of the activation protrusion, based on a spring force, and thus generate the contact pressure. When opening the second door element, a dead center of the spring element can be able to be overcome in order to be able to open the second door element. When closing the second door element, the dead center of the spring element can be able to be overcome again in order to be able to close the second door element, whereby the activation contour can then exert the contact pressure again based on the spring force.

It can be provided, for example, that the part proximal to the center of rotation is designed as and/or comprises a first magnetic element, and the part proximal to the object is designed as and/or comprises a second magnetic element. For example, the magnetic elements can rest on one another, or can be mutually adjacent, when the second door element is closed, so that the contact pressure and/or maintaining tightness is possible based on a magnetic force. When opening the second door element, the magnetic elements can be able to be brought to a mutual spacing via a pivoting movement of the center of rotation, in particular after overcoming the magnetic force and/or after corresponding de-energizing. It is therefore understood that the magnetic elements can be designed as a permanent magnet element and/or solenoid element.

It can be provided, for example, that the contact pressure apparatus for generating a contact pressure and/or for maintaining tightness of the second door element in relation to the wall portion and/or access opening assigned to the second door element comprises one or a plurality of magnetic elements.

Preferably, the magnetic elements can be disposed on and/or in the assigned wall portion of the second object, in particular of the second process unit, preferably so as to be adjacent to and/or on the assigned access opening.

For example, the magnetic elements can be disposed externally on the assigned wall portion.

It can be provided that the second door element is partially or completely designed as a magnetic counter-element or comprises at least one such magnetic counter-element.

For example, a respective counter-element comprised by the second door element can be disposed so as to correspond to said magnetic elements of the contact pressure apparatus.

For example, due to their corresponding disposal, the magnetic elements and the one or more counter-elements can rest on one another or be disposed mutually adjacent when the second door element is closed, or is received in the corresponding access opening, so that the contact pressure and/or maintaining tightness are/is able to be effected based on a magnetic force.

Said counter-element can be able to be manufactured, or be manufactured, for example, from a magnetizable metal or alloy, e.g. magnetizable steel.

Additionally or alternatively, said counter-element can be able to be manufactured, or be manufactured, for example, from a magnetic material. It can be conceivable, for example, that this counter-element is fixed in and/or on the respective door element, for example in an in particular integrated enclosure, e.g. stainless steel enclosure, of the respective door element and/or by means of overmolding with a plastics material, e.g. epoxy resin. This can advantageously enable a hygienically compliant design embodiment, in particular a hygienic design.

For example, a magnetic element can be designed as a solenoid. It can be conceivable, for example, that the latter can effect the contact pressure and/or maintaining tightness when de-energized. In other words, energizing can initiate a release of the second door element.

It can be provided that the first door element is able to be manufactured, or is manufactured, partially or completely from a plastics material, preferably from a plastics material capable of being sterilized and/or autoclaved, for example polyether ether ketone (PEEK).

It can be provided that the second door element is able to be manufactured, or is manufactured, partially or completely from a plastics material, preferably from a plastics material capable of being sterilized and/or autoclaved, for example polyether ether ketone (PEEK).

It can be provided that the opener apparatus and in particular the lever element is able to be motorized, or is motorized, for said opening and closing.

For example, a motor apparatus can be provided for this purpose, which can be able to be disposed, or can be disposed, in and/or on the second object, in particular the second process unit, and can optionally be comprised by the transfer device, in particular the opener apparatus.

The motor apparatus may, for example, be part of the said drive unit and/or be comprised by the latter.

The opener apparatus, in particular the lever element, can be actuated in an in particular automated manner by means of the motor apparatus.

The motor apparatus can be actuatable by means of a, or the, control unit of the transfer device and/or by means of a, or the, control unit which is assignable, or is assigned, to the transfer device.

Additionally or alternatively, it can be provided that the pivotable lever element is formed from a plurality of, e.g. two or more, lever links which can be in particular movably connected to one another.

For example, the pivotable lever element can be formed from a first and a second lever link. In the installed state, the first lever link can be pivotably fixed in the second object, in particular the second process unit, on a first end region of the first lever link, and a first end region of the second lever link can be pivotably fixed on a second end region of the first lever link. The second door element can be pivotably fixed on a second end region of the second lever link.

For example, a third lever link of the lever element can be disposed, and in particular pivotably fixed, between the second lever link and the second door element. A first end region of the third lever link can be pivotably fixed on the second end region of the second lever link, and a second end region of the third lever link can be pivotably fixed on the second door element. Preferably, the first, the second and the third lever link can be disposed in, and/or form, a parallelogram arrangement and/or a parallelogram guide. The parallelogram arrangement and/or parallelogram guide can be advantageously space-saving in the interior of the second object, in particular of the second process unit.

By means of the lever links, the second door element, or the mutually engaged door elements conjointly, can be able to be moved into the interior of the second object, in particular of the second process unit, and thus be able to be removed from the access openings, in particular by a movement in a plane, advantageously by a one-dimensional and/or two-dimensional linear movement in a plane.

By means of the parallelogram arrangement and/or parallelogram guide, the second door element, or the mutually engaged door elements conjointly, can be moved into the interior of the second object, in particular of the second process unit, and thus be removed from the access openings, in particular by a movement in a plane, advantageously by a one-dimensional and/or two-dimensional linear movement in a plane.

It can be provided that a first motor is disposed between the second object, in particular the second process unit, and the first lever link, and a second motor is disposed between the first lever link and the second lever link. The first and the second motor can form a, or said, motor apparatus and/or be comprised by the latter.

Additionally or alternatively, it can be provided that a motor is disposed between the second object, in particular the second process unit, and the first lever link, and/or the first lever link is connected by way of the motor to the second object, wherein the first lever link and the second lever link can form a lever kinematics in such a way that the second door element disposed on the second lever link, or the mutually engaged door elements conjointly, is/are movable linearly into the interior of the second object, in particular of the second process unit, and subsequently is/are pivotable in the interior in such a way that an unobstructed access to the assigned access opening can be enabled.

It can be provided that a first and a second motor are disposed between the second object, in particular the second process unit, and the lever element, wherein the second motor is disposed between the first motor and the lever element, and the first motor is disposed between the second motor and the second object. In other words, the following arrangement can be provided: second object, first motor, second motor, lever element. The first and the second motor can form a, or said, motor apparatus and/or be comprised by the latter. For example, the first motor can be a motor for initiating a linear movement, e.g. a spindle drive motor, and/or the second motor can be a motor for initiating a rotating movement, e.g. a rotational motor.

It can be provided that the first motor can move the second door element, or the mutually engaged door elements conjointly, linearly in the direction of the interior of the second object, in particular of the second process unit, and, in particular subsequently, the second motor can pivot the second door element, or the mutually engaged door elements conjointly, in the interior, preferably for an opening procedure. It can be provided that a closing procedure can take place in the reverse order.

It can be provided that a first and a second motor are disposed between the second object, in particular the second process unit, and the lever element, wherein the second motor is disposed between the first motor and the lever element, and the first motor is disposed between the second motor and the second object. In other words, the following arrangement can be provided: second object, first motor, second motor, lever element. The first and the second motor can form a, or said, motor apparatus and/or be comprised by the latter. For example, the first motor and the second motor can each be a motor for initiating a rotating movement, e.g. a rotational motor. For example, the first and the second motor can be disposed transversely and in particular perpendicularly to one another; in other words, their respective centers of rotation can be disposed transversely and in particular perpendicularly to one another.

For example, it can be provided that the lever element is disposed and/or fixed centrically or eccentrically on the second motor relative to a, or the, center of rotation of the second motor.

It can be provided that the second motor can rotate the second door element, or the mutually engaged door elements conjointly, in the direction of the interior of the second object, in particular of the second process unit, and, in particular subsequently, the first motor can pivot the second door element, or the mutually engaged door elements conjointly, in the interior, preferably for an opening procedure. It can be provided that a closing procedure can take place in the reverse order.

For example, it can be provided that the lever element is connected to the second motor by way of a guide kinematics, in particular a parallelogram kinematics, so that a rotating movement which is able to be initiated, or is initiated, by the second motor can cause a circular movement of the lever element and thus of the second door element, or the mutually engaged door elements, away from the access opening. Preferably, at least the second door element, or the mutually engaged door elements, can remain so as to be aligned substantially parallel to the access opening during this circular movement.

Additionally or alternatively, it can be provided that the pivotable lever element is formed from one or a plurality of, e.g. two, lever links, which can be in particular movably connected to one another, wherein the pivotable lever element, and thus at least partially the opener apparatus, can form, and/or comprise, a part of a handling apparatus.

A lever link which is assigned, or assignable, to the second door element can be releasably connected to the second door element, in particular by way of a gripper actuator mechanism and/or coupling apparatus configured on this lever link.

The handling apparatus can be or comprise a multi-axis robot arm.

Additionally or alternatively, it can be provided that the handling apparatus is, or comprises, a pick-and-place robot or a cable robot, and/or that the handling apparatus is, or comprises, a suitable gripper actuator mechanism configured on a planar stage.

Additionally or alternatively, it can be provided that the handling apparatus in the installed state in the second object, in particular the second process unit, is guided by rail for a wide and in particular larger effective range and in particular handling range.

It can be provided that by means of the handling apparatus, the second door element, or the mutually engaged door elements conjointly, is/are movable into the interior of the second object, in particular of the second process unit, and thus able to be removed from the access openings.

For example, it can be provided that by means of the handling apparatus, the second door element, or the mutually engaged door elements conjointly, are movable, preferably in the manner of a plug, out of the access openings and back into the latter, in particular into the interior of the second object, in particular of the second process unit, and thus is/are removable from the access openings, and also able to be moved back into them.

It can be provided, for example, that a linear guide and/or a pull-out guide, e.g. telescopic guide, is/are disposed on the access opening of the second object, in particular of the second process unit, along which guide the second door element, or the mutually engaged door elements conjointly, is/are able to be moved, preferably in the manner of a drawer, out of the access openings and back into the latter, in particular into the interior of the second object, in particular of the second process unit, and thus removable from the access openings, and also able to be moved back into them, by means of the handling apparatus. For example, the second door element can be able to be coupled, or can be coupled, to a displacement device, e.g. carriage and/or cart, the linear guide and/or the pull-out guide.

Furthermore, it is understood that further second door elements, or further mutually engaged door elements conjointly, is/are able to be moved into the interior of the second object, in particular of the second process unit, and thus removable from the access openings, by means of the handling apparatus.

It can be provided that the opener apparatus has at least one guide apparatus and a motor apparatus, wherein the second door element is fixed on the guide apparatus and the guide apparatus is fixed adjacent to the inlet opening, and wherein the second door element is movable, in particular linearly, from the associated access opening along a part of the guide apparatus, and subsequently movable, in particular linearly, along a further part of the guide apparatus away from the associated access opening, by means of the motor apparatus.

For example, the second door element can be moved out of the associated access opening by a movement which lies in a plane which can be oriented transversely and in particular perpendicularly to the associated access opening, or to the plane defined by the latter.

For example, the second door element can be moved away from the associated access opening by a movement which lies in a plane which can be oriented transversely and in particular perpendicularly to the previous movement.

For example, vectors of the respective movements can be transverse, in particular perpendicular, to one another.

For example, the movement from the access opening can be oriented substantially horizontally, and the subsequent movement away from the access opening can be oriented substantially perpendicularly.

It can be provided that the second door element is able to be moved in an analogous manner back into the access opening; in other words, it is movable back along the previously described movement path.

For example, it can be provided that the guide apparatus comprises a first guide element and a second guide element, which are in each case disposed on mutually opposite sides of the access opening in the interior of the second object, in particular of the second process unit.

Preferably, the guide elements can be disposed parallel to one another and/or disposed in a common plane, e.g. in a plane parallel to the access opening.

Preferably, the second door element can be movably held on the guide elements by way of a holding kinematics of the guide apparatus.

For example, the second door element can be movably held on the guide elements by way of a plurality of holding links of the holding kinematics.

For example, the second door element can be moveably held on a respective guide element by way of one, two, three or more holding links.

For example, a number of holding links per guide element, by way of which the second door element can be movably held on the guide elements, can be identical or different.

It can be provided that the second door element is able to be moved, in particular linearly, out of the access opening and/or back into the latter by way of the holding kinematics by means of the motor apparatus.

Additionally or alternatively, it can be provided that the second door element is able to be moved, in particular linearly, out of the access opening and/or back into the latter by means of one or a plurality of eccentric apparatuses which are in each case configured and/or disposed on the guide elements and which can be activatable by means of the motor apparatus. In other words, the second door element can be movable conjointly with the guide elements, in particular linearly, away from the access opening and/or toward the access opening by means of the one or the plurality of eccentric apparatuses of the guide elements.

It can be provided that the second door element, in particular in a state moved out of the access opening, can be movable away from the access opening by way of the guide elements.

It can be conceivable that a guide element is designed as, and/or comprises, a guide rail such as, for example, a magnetic rail on which the second door element can be movably held based on a magnetic force.

For example, the holding links of the holding kinematics can in each case be designed as counter-elements of the magnetic rail, and/or function as such and/or have such.

The holding links can be designed to be, for example, at least in part in the manner of slides and/or carriages.

As already described, it can be provided that the opener apparatus has a, or the, drive unit which is disposed in and/or on the second object, in particular the process unit, wherein the lever element, and thus also the second door element (by way of the lever element), is fastened to the drive unit.

By means of the opener apparatus, and in particular initiated by the drive unit, the second door element, or the mutually engaged door elements conjointly, is/are able to be moved in a pivoting and linear manner into the interior of the second object, in particular of the second process unit, and thus removable out of and/or from the access opening(s).

In other words, it can be provided that the drive unit can move the second door element, or the mutually engaged door elements conjointly, linearly in the direction of the interior of the second object, in particular of the second process unit, and, in particular subsequently, can pivot in the interior, preferably for an opening procedure.

It can be provided in particular that the drive unit can pivot the second door element, or the mutually engaged door elements conjointly, in the interior of the second object, in particular of the second process unit, and, in particular subsequently, can move the second door element, or the mutually engaged door elements conjointly, linearly in the direction of the access opening and preferably into the latter. This could be a closing procedure, for example. The pivoting herein can take place, for example, until the second door element, or the mutually engaged door elements, are congruent with the access opening(s), preferably so that they can be returned to the access openings.

It can be provided that the drive unit has a motion apparatus and a motor apparatus, e.g. the motor apparatus already described, wherein the motion apparatus is rotatable and at least partially linearly movable by means of the motor apparatus.

It can be provided, for example, that the motion apparatus has a center of rotation, e.g. the center of rotation already described, which is preferably formed as a shaft, e.g. hollow shaft, and has a displacement device, wherein the displacement device is disposed externally on the center of rotation and is linearly movable and/or displaceable along the center of rotation.

For example, the displacement device can comprise a plurality of roller elements for rolling on the center of rotation and/or one or a plurality of sliding elements for sliding on the center of rotation, in particular for enabling linear mobility of the displacement device along the center of rotation.

For example, the displacement device can be designed in the manner of a carriage and/or can have at least one carriage unit.

For example, the displacement device can be designed in the manner of a slide and/or can have at least one slide unit.

For example, the displacement device can externally surround the center of rotation in portions and/or completely.

Preferably, the center of rotation can be disposed transversely and in particular perpendicularly to the wall of the second object.

The center of rotation and the displacement device can be rotatable, and/or the displacement device can be linearly movable and/or displaceable, by means of the motor apparatus.

For example, the motor apparatus can have a rotational motor for initiating a rotation movement of the motion apparatus and a spindle drive motor for initiating a linear movement of at least one part of the motion apparatus, in particular the displacement device.

The center of rotation and the displacement device can be rotatable by means of the rotational motor, and/or the displacement device can be able to be initiated for linear movement by means of the spindle drive motor.

It can be provided that the rotational motor is disposed externally on the center of rotation and/or is fastened indirectly or directly to the wall of the second object.

It can be provided that the spindle drive motor is disposed in the center of rotation. Alternatively, it can be provided that the spindle drive motor is disposed on the center of rotation.

It can be provided that an in particular slide-like and/or carriage-like displacement element which is comprised by the motion apparatus, and which engages with a spindle of the spindle drive motor and is linearly movable and/or displaceable along the spindle within the center of rotation due to the rotation of the spindle drive motor, is disposed in the center of rotation.

It can be provided that the displacement element and the displacement device are operatively connected to one another in a contactless, preferably magnetic, manner, in particular for conjoint mobility and preferably for conjoint mobility along the center of rotation.

Thus, when the displacement element moves, preferably linearly, it can be provided that the displacement device correspondingly moves, preferably linearly, in particular along the center of rotation.

For example, the displacement element can have one or a plurality of entrainment magnets, and the displacement device can have one or a plurality of corresponding magnetic and/or magnetizable counter-elements, as a result of which a contactless operative connection between the displacement element and the displacement device can be enabled.

It can be provided that the motor apparatus has a braking apparatus which is disposed on the center of rotation, preferably on an end thereof and/or adjacent to the rotational motor, and by means of which a rotating movement of the center of rotation can be able to be braked, in particular decelerated and/or stopped.

It can be provided that the drive unit is disposed partially or completely in the interior of the second object, in particular of the second process unit. It can be provided that the drive unit is disposed partially outside the interior of the second object, in particular of the second process unit, for example externally and/or in a separate interior space of the second object.

For example, it can be conceivable that the center of rotation extends through the wall of the second object, in particular of the second process unit, in particular so as to be sealed in relation to the outside. Preferably, that part of the center of rotation on which the displacement device is disposed can be disposed in the interior of the second object, in particular of the second process unit.

For example, that part of the center of rotation on which the rotational motor and/or the braking apparatus and/or the spindle drive motor is/are disposed can be disposed outside the interior of the second object, in particular of the second process unit, in other words disposed externally on the latter.

For example, the center of rotation can be mounted on and/or in and/or adjacent to the wall by means of at least one bearing device.

It can be provided that the transfer device comprises one or a plurality of sensor units for detecting an open state of the first door element and/or for detecting an open state of the second door element.

It can be provided that each sensor unit is connected, in particular for signaling, to a, or the, control unit of the transfer device and/or a, or the, control unit assignable, or assigned, to the transfer device, in particular for detecting the open state of the first door element and/or for detecting the open state of the second door element, and furthermore in particular for determining whether transitioning between the objects, in particular the first and second process units, is enabled.

It can be provided that the open state of the first door element and/or the open state of the second door element is/are detectable by means of detecting the position of the first door element and/or of the second door element by the one, or the plurality of, sensor unit(s).

In other words, a sensor unit can be designed as, and/or comprise, a position detection sensor.

Additionally or alternatively, it can be provided that the open state of the first door element and/or the open state of the second door element is/are able to be detected by the one, or the plurality of, sensor unit(s) based on torque and/or force.

In other words, a sensor unit can be designed as, and/or comprise, a torque sensor and/or force sensor.

Additionally or alternatively, it can be provided that the open state of the first door element and/or the open state of the second door element is/are detectable inductively and/or optically and/or capacitively and/or magnetically by the one, or the plurality of, sensor unit(s). In other words, a sensor unit can be designed as, and/or comprise, a sensor operating on the basis of the aforementioned principles. For example, a sensor unit can be designed as, and/or comprise, a pressure sensor and/or a distance sensor.

Additionally or alternatively, it can be provided that the open state of the first door element and/or the open state of the second door element is/are detectable by means of in particular mechanically or electrically detecting the contact on the first door element and/or on the second door element relative to the corresponding object, in particular the corresponding process unit, in particular the access opening of the corresponding object, in particular of the corresponding process unit, by the one, or the plurality of, sensor unit(s).

Additionally or alternatively, it can be provided that the transfer device comprises at least one sensor unit by means of which it is detectable whether the first door element and the second door element are engaged with one another.

Additionally or alternatively, it can be provided that a mass moved by the opener apparatus during opening and/or closing is detectable by means of the at least one sensor unit.

Additionally or alternatively, it can be provided that a change in a magnetic field in and/or on the second object, in particular the second process unit, which change is able to be caused by the opener apparatus when opening and/or closing, is detectable by means of the at least one sensor unit.

Additionally or alternatively, it can be provided that an electrical contact, which is closed in the engaged state of the first and the second door element, is detectable by means of the at least one sensor unit.

Additionally or alternatively, it can be provided that an air pressure between the first and second door element brought into engagement is able to be provided by means of the at least one sensor unit, and this air pressure is detectable for checking a tightness between the first and the second door element.

It can be provided that the at least one sensor unit is connected, in particular for signaling, to a, or the, control unit of the transfer device, and/or a, or the, control unit assignable, or assigned, to the transfer device, for determining whether the first door element and the second door element are mutually engaged.

It can be conceivable that one or a plurality of sensor units is/are provided on the center of rotation assigned to the opener apparatus, by means of which the open state of the first door element and/or the open state of the second door element is/are detectable, and/or it is detectable whether the first door element and the second door element are engaged with one another. In addition to or as an alternative to the previously mentioned sensor units, it is conceivable here for example that a sensor unit can detect a rotation of the center of rotation based on a motor current, in particular by means of the motor apparatus, designed in particular as a pivot motor, by means of which the opener apparatus and in particular the lever element and/or the center of rotation can be motorized.

It is understood that a detection of the respective open states can additionally comprise an opening and/or closing procedure of said door elements.

It is furthermore understood that a sensor unit for detecting an open state of the first door element and/or for detecting an open state of the second door element, and a sensor unit for detecting whether the first door element and the second door element are mutually engaged, can be implemented in one sensor unit or in a plurality of sensor units which differ from one another, for example. It goes without saying that any suitable combination of sensor units can be conceivable here.

It can be provided that the first door element comprises a holding contour which, in the closed state, is disposed internally relative to the first object, in particular the first process unit, for example disposed in the interior of the first object, in particular of the first process unit, and faces the latter.

It can be provided that the first door element comprises a holding portion which is disposed on the, in particular truncated conical or truncated pyramidal, portion of the first door element and extends away from the latter, in particular in the closed state of the first door element extends into the interior of the first object, in particular of the first process unit.

For example, the holding portion can be substantially cylindrical in shape, and said holding contour can be formed on and/or in the holding portion.

Preferably, the holding contour can be designed in the form of a depression, for example a recess and/or groove, in a shell surface of the holding portion and/or in the form of a protrusion on the shell surface of the holding portion.

For example, the holding portion, which has a holding contour formed as a depression, can be substantially mushroom-shaped and/or, in a cross-sectional view, substantially T-shaped or H-shaped.

It can be provided that the locking apparatus comprises at least one holding element which in the installed state is disposed in the first object, in particular the first process unit, and which can be selectively brought to engage with the holding contour in order to lock the first door element.

It should be understood that the at least one holding element can comprise exactly one holding element or more than one holding element. For example, a plurality of holding elements can be provided, which can be disposed at regular or irregular spacings about the holding contour, in order to be able to be selectively brought to engage with the holding contour in order to lock the first door element.

It can be provided that the locking apparatus comprises a drive apparatus by means of which the or each holding element is movable in order to be able optionally to engage it with the holding contour.

In particular, the or each holding element can be movable by means of the drive apparatus between a locked position and a release position.

The locked position can correspond with a state of the holding element engaged with the holding contour, and the release position can correspond with a state of the holding element disengaged from the holding contour.

It can be provided, for example, that the locking apparatus comprises at least one elastic element, in particular spring element, by means of which the or each holding element can be able to be preloaded, or is preloaded, into a, or the, engaged state, wherein the drive apparatus can initiate the or each holding element to move counter to the spring force of the elastic element.

It can be provided, for example, that an assigned elastic element, in particular spring element, for in each case providing said preload is provided on each holding element.

It can be provided, for example, that the elastic element is disposed between a plurality of, for example two, holding elements in order to provide said preload conjointly on the respective holding elements.

It can be provided, for example, that the locking apparatus comprises at least one elastic element, in particular spring element, and at least two holding elements formed as clamping levers, between which the elastic element is disposed, in particular in order to preload the holding elements into a, or the, engaged state. For example, the holding elements can be rotatably mounted, wherein the elastic element is fixed at a respective end region of the holding elements, and a respective opposite end region of the holding elements relative to the mounting serves for engagement. For example, the elastic element can be a compression spring. The elastic element can preload the end regions on which it is fixed into a mutually spaced apart state, and therefore preload, in particular by way of the mounting, the other end regions into the engaged state.

It can be provided, for example, that the locking apparatus comprises one or a plurality of holding elements and a number of elastic elements corresponding to the holding elements, in particular spring elements, wherein a respective elastic element is assigned to a holding element and loads the respective assigned holding element into a, or the, engaged state. For example, the holding elements can be rotatably mounted. For example, the elastic element can engage on the holding element so as to be spaced apart from the mounting. For example, the elastic element can be a compression spring.

It can be provided, for example, that the locking apparatus comprises one or a plurality of holding elements and a bell-crank lever apparatus by means of which the holding elements are able to be maintained in a, or the, engaged state. Optionally, the bell-crank lever apparatus can comprise at least one elastic element, in particular spring element, by means of which the bell-crank lever apparatus is able to be preloaded, or is preloaded, into the state in which the holding elements are in the engaged state. It can be conceivable, for example, that the bell-crank lever apparatus is activatable in a spindle-operated and/or eccentrically operated, e.g. eccentric disk-operated, and/or lever-operated and/or linear actuator-operated and/or rotational actuator-operated and/or rack-and-pinion operated manner, in order to move the holding elements into and/or out of the engaged state.

It can be provided, for example, that the locking apparatus comprises an articulated assembly comprising a plurality of links, and at least two mutually different elastic elements, in particular spring elements, for example tension and/or compression spring elements, and one or a plurality of holding elements, wherein the holding elements are able to be maintained in a, or the, engaged state by way of the articulated assembly, and the articulated assembly is able to be preloaded, or is preloaded, into the state in which the holding elements are in the engaged state by means of the elastic elements.

It can be provided that the drive apparatus in the installed state is disposed in and/or on the first object, in particular the first process unit.

For example, the drive apparatus in the installed state can be disposed in the interior of the first object, in particular of the first process unit, and/or on a wall defining the interior of the first object, in particular of the first process unit, in particular the already described shell wall, of the first object, in particular of the first process unit.

It can be provided that the transfer device and in particular the locking apparatus comprise at least one sensor unit for detecting a locked state of the first door element, by means of which a position of the holding element relative to the drive apparatus and/or the holding contour is detectable.

For example, the locked state of the first door element can be detectable by the at least one sensor unit by means of a position detection of the holding element relative to the drive apparatus and/or the holding contour and/or the first object, in particular the first process unit, by way of which the position of the holding element is able to be determined.

For example, the locked state of the first door element can be detectable by the at least one sensor unit by means of an in particular mechanical or electrical contact detection between the holding element and the drive apparatus and/or the holding contour and/or the first object, in particular the first process unit, by way of which the position of the holding element is able to be determined.

It can be provided that the at least one sensor unit is connected, in particular for signaling, to a, or the, control unit of the transfer device and/or a, or the, control unit assignable or assigned to the transfer device, for detecting the locked state of the first door element.

It can be provided that the drive apparatus comprises an elastic element, in particular spring element, by means of which the or each holding element is able to be preloaded, or is preloaded, in the direction of engagement with the holding contour, and in particular in the direction of the locked position.

It can be provided that the drive apparatus is operatively connected to the holding element by way of a shaft, and by way of which shaft the holding element is able to be brought to engage with the holding contour and brought back out of the latter, and in particular is movable into the locked position and the release position.

In other words, in the case of a plurality of holding elements, the drive apparatus can be operatively connected to respective holding elements by way of a respective shaft, and by way of which shafts the holding elements are able to be brought to engage with the holding contour and brought back out of the latter, and in particular are movable into the locked position and the release position.

It can be provided that the drive apparatus comprises a motor which is operatively connected to the or each shaft and thus to the or each holding element, and by means of which a force and/or a torque is able to be generated, by way of which force and/or torque the or each holding element is able to be initiated to move in order to selectively move the or each holding element into the locked position or into the release position.

It can be provided that the drive apparatus comprises a motor and a positive-ratio gearbox by way of which a torque able to be initiated by the motor, and/or a force able to be initiated by the motor, is able to be delivered, in particular able to be delivered to the shaft, in order to bring the holding element to engage with the holding contour, and in particular to move the holding element into the locked position and the release position.

It can be provided that the drive apparatus comprises a, in particular the, motor and a spindle drive driven by means of the motor, and formed on the shaft is an activating arm which is movably connected to the spindle drive and by way of which the holding element is able to be initiated to move in order to selectively move the holding element into the locked position or into the release position.

Preferably, a rotating movement of the spindle drive can be able to be transmitted to the shaft in the form of a pivoting movement by way of the connection of the shaft to the activating arm, which pivoting movement is able to be transmitted by way of the shaft to the holding element.

In other words, it can be provided that the drive apparatus comprises a, or the, motor and a spindle drive driven by means of the motor, and formed on the shaft is an activating arm which is movably connected to the spindle drive, wherein a rotating movement of the spindle drive is able to be transmitted to the shaft in the form of a pivoting movement by way of the connection of the shaft to the activating arm, which pivoting movement is able to be transmitted by way of the shaft to the holding element.

As previously described, the locking apparatus can comprise a plurality of holding elements. It is to be understood in this context that the relevant explanations pertaining to the at least one holding element can also apply in an analogous manner to each holding element.

Preferably, the locking apparatus can comprise, for example, a further, e.g. second, holding element which is movably connected to the spindle drive by way of a further, e.g. second, shaft by means of a further, e.g. second, activating arm formed on the further shaft. It is to be understood that the previously described holding element and the components connected thereto, such as the shaft and/or the activating arm, can correspondingly be referred to as the first holding element and as the first corresponding component.

It can be provided that the two holding elements can be brought to engage with the holding contour, and brought back out of the latter, and in particular are movable into the locked position and the release position, by means of a mutually opposing and in particular synchronous pivoting movement.

It can be provided that the drive apparatus and in particular the motor of the drive apparatus are actuatable by means of a, or the, control unit of the transfer device and/or by means of a, or the, control unit assignable or assigned to the transfer device, in particular in order to be able to initiate the or each holding element to move, and in particular in order to be able to move the latter into the locked position and the release position.

Additionally or alternatively, it can be provided that the locking apparatus comprises one or a plurality of magnetic elements.

Preferably, the magnetic elements can be disposed on and/or in the assigned wall portion of the first object, in particular of the first process unit, preferably adjacent to and/or on the assigned access opening.

For example, the magnetic elements can be disposed internally, or in the interior of the first object, in particular of the first process unit, on the assigned wall portion.

It can be provided that the first door element is designed partially or completely as a magnetic counter-element, or comprises at least one of the latter.

For example, a respective counter-element comprised by the first door element can be disposed so as to correspond to said magnetic elements of the locking apparatus.

For example, due to the corresponding arrangement, the magnetic elements and the counter-element(s) can rest on one another or be mutually adjacent when the first door element is closed, or is received in the corresponding access opening, so that a contact pressure and/or maintaining tightness based on a magnetic force is advantageously able to be effected.

Said counter-element can be able to be manufactured, or be manufactured, for example, from a magnetizable metal or alloy, e.g. magnetizable steel.

Additionally or alternatively, said counter-element can be able to be manufactured, or be manufactured, for example, from a magnetic material. It can be conceivable, for example, that this counter-element is fixed in and/or on the respective door element, for example in an in particular integrated enclosure, e.g. stainless steel enclosure, of the respective door element and/or by means of overmolding with a plastics material, e.g. epoxy resin. This can advantageously enable a hygienically compliant design embodiment, in particular a hygienic design.

For example, a magnetic element can be designed as a solenoid. It can be conceivable, for example, that the latter can effect the contact pressure and/or maintaining tightness when de-energized. In other words, energizing can initiate a release of the first door element.

It can be provided, for example, that the locking apparatus can function as a contact pressure apparatus for generating a contact pressure and/or for keeping the first door element tight in relation to the wall portion and/or access opening assigned to the first door element, and/or can form such a contact pressure apparatus.

It can preferably be provided that the magnetic elements of the locking apparatus and/or contact pressure apparatus of the first door element are spaced apart from the magnetic elements of the locking apparatus and/or contact pressure apparatus of the second door element, so as to allow a mutual spacing of their magnetic fields.

It can be provided that the respective magnetic elements of the one locking apparatus and/or contact pressure apparatus are provided in a substantially vertical or a substantially horizontal arrangement on the respective assigned wall portion, and the respective magnetic elements of the other locking apparatus and/or contact pressure apparatus in a transversely, in particular perpendicularly, oriented arrangement on the respective assigned other wall portion.

Additionally or alternatively, it can be provided that the magnetic elements of the locking apparatus and/or contact pressure apparatus assignable or assigned to the first and/or the second door element are in each case disposed equidistantly from the respective access opening.

It can be provided that the transfer device comprises a further locking apparatus for locking the second door element in a closed state and for releasing the second door element for opening the latter.

The further locking apparatus in the installed state can be disposed in and/or on the second object, in particular the second process unit.

It can be provided, for example, that the first door element and the second door element are able to be brought to engage with one another, and in the engaged state, when the first door element is released by means of the locking apparatus and the second door element is released by means of the further locking apparatus, are able to be removed conjointly from the access openings by means of the opener apparatus, in order to enable the in particular aseptic transition between the objects, in particular the first process unit and the second process unit.

It can be provided, for example, that the further locking apparatus is formed as the already described contact pressure apparatus which comprises one or a plurality of magnetic elements. In all other respects, reference can be made here to the respective description of the contact pressure apparatus comprising the magnetic element(s).

It can be provided, for example, that the further locking apparatus is designed as, and/or comprises, a bayonet apparatus. Male and/or female bayonet elements can be provided in and/or on the second door element and/or the opener apparatus, in particular the lever element. It is understood that corresponding female and/or male bayonet counter-elements can be able to be disposed, or in the installed state can be disposed, in and/or on the second object, in particular the second process unit, in order to be able to be brought to engage with the bayonet elements. The bayonet apparatus and in particular the bayonet elements and/or the bayonet counter-elements can be motorized in order to be able to bring the bayonet elements and/or bayonet counter-elements to engage, in particular in a rotating manner, so as to lock and/or release the second door element. It can be provided, for example, that the motorized bayonet elements and/or bayonet counter-elements are actuatable by means of a control unit of the transfer device and/or by means of a control unit assignable or assigned to the transfer device, in particular so as to lock and/or to release the second door element.

It can be provided, for example, that the further locking apparatus is designed as, and/or comprises, a clamping and/or tensioning apparatus. For example, at least one clamping and/or tensioning lever can be able to be disposed, or in the installed state can be disposed, in and/or on the second object, in particular the second process unit, in order to be able to be brought to engage with the second door element and/or the opener apparatus, in particular the lever element, and to exert on those selectively a force directed toward the access opening of the second object, in particular of the second process unit, so as to lock the second door element. The clamping and/or tensioning apparatus and in particular the clamping and/or tensioning lever can be motorized to bring the clamping and/or tensioning lever to engage with the second door element and/or the opener apparatus, in particular the lever element. It can be provided, for example, that the motorized clamping and/or tensioning apparatus and in particular the motorized clamping and/or tensioning lever by means of a control unit of the transfer device and/or by means of a control unit assignable or assigned to the transfer device can be controlled, in particular for locking and/or for releasing the second door element.

It can be provided, for example, that the further locking apparatus is designed as, and/or comprises, a bell-crank lever apparatus, which, for example, is able to be disposed, or in the installed state is disposed, in and/or on the second object, in particular the second process unit, in order to keep the second door element in a, or the, locked state. Optionally, the bell-crank lever apparatus can comprise an elastic element, in particular a spring element, by means of which the second door element is able to be preloaded, or is preloaded, into the locked state. The bell-crank lever apparatus can be connected to the second door element and/or the opener apparatus, in particular the lever element, and in the installed state can be connected to the second object, in particular the second process unit. The spring element can be connected to the remainder of the bell-crank lever apparatus, and in the installed state can be connected to the second object, in particular the second process unit. For example, a force directed toward the access opening of the second object, in particular of the second process unit, for locking the second door element can be able to be exerted by means of the bell-crank lever apparatus.

It can be provided, for example, that the further locking apparatus is designed as, and/or comprises, an eccentric apparatus. The eccentric apparatus can comprise at least one shaft on which an eccentric element is formed. By means of a rotation of the shaft, a portion of the eccentric element which is eccentric relative to the shaft can be able to be brought to engage with the second door element and/or the opener apparatus, in particular the lever element, for locking the second door element, in particular in order to be able to exert on those a force directed toward the access opening of the second object, in particular of the second process unit, so as to lock the second door element. The eccentric apparatus and in particular the shaft can be motorized in order to be able to bring the eccentric element to engage with the second door element and/or the opener apparatus, in particular the lever element. It can be provided, for example, that the motorized eccentric apparatus and in particular the motorized shaft are actuatable by means of a control unit of the transfer device and/or by means of a control unit assignable or assigned to the transfer device, in particular for locking and/or for releasing the second door element.

It can be provided that the transfer device and in particular the further locking apparatus comprise at least one sensor unit for detecting a locked state of the second door element.

For example, the locked state of the second door element can be detectable by the at least one sensor unit by means of position detection.

For example, the locked state of the second door element can be detectable by the at least one sensor unit by means of an in particular mechanical or electrical contact detection between the second door element and the second object, in particular the second process unit, and in particular the respective access opening.

For example, the locked state of the second door element can be detectable inductively and/or optically and/or capacitively by the at least one sensor unit.

It can be provided that the at least one sensor unit is connected, in particular for signaling, to a, or the, control unit of the transfer device and/or a, or the, control unit assignable or assigned to the transfer device, in order to detect the locked state of the second door element.

It can furthermore be provided that the transfer device comprises one or a plurality of sensor units for detecting foreign matter in the respective access openings of the objects, in particular of the first and the second process unit.

For example, the presence of foreign matter in the respective access openings can be detectable optically, e.g. in a camera-based and/or light barrier-based, and/or force-based and/or position-based manner, by means of said sensor units.

It can also be provided that the transfer device comprises one or a plurality of sensor units for detecting a contact pressure of the first and/or the second door element in the respective access openings of the objects, in particular of the first and the second process unit.

For example, the contact pressure can be detectable in a torque-based and/or force-based and/or motor current-based manner by means of said sensor units.

It can be provided that the drive apparatus in the installed state is disposed on the first object, in particular the first process unit, and outside the interior of the first object, in particular of the first process unit.

It can be provided that the drive apparatus in the installed state is disposed within the first object, in particular the first process unit, wherein the drive apparatus is partially or completely disposed in a separate interior space of the first object, in particular of the first process unit, which differs from the interior of the first object, in particular of the first process unit, and, optionally, is partially disposed in the interior of the first object, in particular of the first process unit.

Alternatively, it can be provided that the drive apparatus in the installed state is disposed within the first object, in particular the first process unit, and is designed to be at least partially autoclavable, wherein an autoclavable part of the drive apparatus and a non-autoclavable part of the drive apparatus are able to be connected, or are connected, to one another by way of at least one coupling component, and the non-autoclavable part of the drive apparatus is able to be releasably installed in the interior of the first object, in particular of the first process unit.

Alternatively, it can be provided that the drive apparatus in the installed state is disposed within the first object, in particular the first process unit, and is designed to be autoclavable, wherein in particular the drive apparatus comprises an in particular sealed housing which is partially or completely, in particular thermally, insulated in relation to its environment.

It can be provided that the drive apparatus in the installed state is disposed within the first object, in particular the first process unit, wherein the drive apparatus is releasably installed, or able to be installed, therein, in particular for selectively installing and removing after and before an autoclaving operation of at least the interior of the first object, in particular of the first process unit.

It can be provided that the transfer device comprises a, or the already described, control unit and/or the transfer device is assignable, or assigned, a, or the already described, control unit, in particular a control unit of the first and/or of the second object, preferably of the first and/or of the second process unit.

It can be provided that this control unit and/or its signal connections are able to be selectively disconnected and/or de-energized for autoclaving of the transfer device and in particular for autoclaving of the respective interior of the objects, in particular of the first and/or of the second process unit.

The present invention furthermore provides a system consisting of at least two objects, in particular process units, and/or of three or four or more objects, in particular process units.

It goes without saying that in said system not every object has to be a process unit. For example, any combination of suitable objects can be provided; for example of one or a plurality of process units and/or one or a plurality of production rooms and/or one or a plurality of conveyor.

It is furthermore to be understood that all structural and/or functional features which are related to the above-described and/or below-described transfer device according to the invention and the embodiments thereof can also be included in said system either alone or in combination, and the associated properties, embodiments and/or advantages can likewise be correspondingly included and achieved.

For example, it can be provided that the system is formed from, and/or comprises, at least two process units.

The two process units can comprise at least one transfer device according to the above and/or following embodiments.

It can be provided that each process unit serves for manufacturing a product, in particular a biological-pharmaceutical product, which has already been described at the outset.

It can be provided that the system is formed from, and/or comprises, three or four or more process units.

In each process unit, one or a plurality of first and/or one or a plurality of second parts of a transfer device according to the above and/or following embodiments can be provided. It is understood that this is conceivable and/or may apply in the context of each object.

The transfer device can be able to be formed from at least one first and at least one second part.

It can be provided that at least one first part and at least one second part of the transfer device are provided in mutually different process units.

A first part can presently be understood to be in particular that part of the transfer device that is assigned to the first door element and/or is connected thereto, or which is able to be disposed, or is disposed, in and/or on the first process unit.

A second part can presently be understood to be in particular that part of the transfer device that is assigned to the second door element and/or is connected thereto, or which is able to be disposed, or is disposed, in and/or on the second process unit.

For example, a process unit can be provided in which one or a plurality of first and/or one or a plurality of second parts of the transfer device are provided, and which is able to be connected to a further process unit, in which at least a first and/or a second part of the transfer device are/is provided, by means of the transfer device, which can be formed from at least one first and at least one second part.

In this way, an in particular aseptic transition between these process units can be enabled, for example for passing through one or a plurality of tool units and/or one or a plurality of sensor units and/or consumables and/or consumable materials.

Referring to the preceding description, a transfer device can be provided with the present invention, by means of which any item, e.g. a treatment object, a tool unit, a sensor unit, a material and/or a substance, is aseptically exchangeable between two objects and preferably between two process units.

Furthermore, the simple and robust configuration of the transfer device can enable a capability for easy cleaning and/or sterilizing, in particular autoclaving.

Moreover, the transfer device can in particular be activatable in a fully automated manner, so that it can facilitate a large-scale application and/or an industrial production of individual and/or very small batches.

Further preferred features and/or advantages of the present invention are the subject of the following description and the graphic representation of exemplary embodiments.

### BRIEF DESCRIPTION OF THE DRAWINGS

FIG. 1 shows a schematic perspective view of a transfer device for enabling an in particular aseptic transition between a first process unit and a second process unit according to an exemplary embodiment of the present invention;

FIG. 2 shows a further schematic perspective view of the transfer device of FIG. 1;

FIG. 3 shows a schematic view of the transfer device of FIG. 1, proceeding from the first process unit in the direction of the second process unit;

FIG. 4 shows a schematic sectional view of the transfer device of FIG. 3 along the section A-A;

FIG. 5 shows a schematic lateral view of the transfer device of FIG. 1;

FIG. 6 shows a schematic top view of a system consisting of at least two process units according to an exemplary embodiment of the present invention;

FIG. 7 shows a schematic top view of a system consisting of three or four or more process units according to an exemplary embodiment of the present invention;

FIGS. 8 to 18 each show a schematic view of a part and/or components of a transfer device for enabling an in particular aseptic transition between a first process unit and a second process unit according to respective further exemplary embodiments of the present invention;

FIG. 19 shows a schematic perspective view of a transfer device for enabling an in particular aseptic transition between a first process unit and a second process unit according to a further exemplary embodiment of the present invention;

FIG. 20 shows a further perspective view of the transfer device of FIG. 19 during an opening procedure;

FIG. 21 shows a further perspective view of the transfer device of FIG. 19 after an opening procedure;

FIG. 22 shows a further perspective view of the transfer device of FIG. 21;

FIG. 23 shows a further perspective view of the transfer device of FIG. 19;

FIG. 24 shows a sectional perspective view of the transfer device of FIG. 19;

FIG. 25 shows a schematic perspective view of a part and/or components of the transfer device of FIG. 19;

FIG. 26 shows a sectional perspective view of a part and/or components of the transfer device of FIG. 19;

FIG. 27 shows a further schematic view of a part and/or components of the transfer device of FIG. 19;

FIG. 28 shows a further sectional perspective view of a part and/or components of the transfer device of FIG. 19;

FIG. 29 shows a sectional detailed view of a part and/or components of the transfer device of FIG. 19;

FIG. 30 shows a further sectional perspective view of a part and/or components of the transfer device of FIG. 19;

FIG. 31 shows a further sectional perspective view of a part and/or components of the transfer device of FIG. 19;

FIG. 32 shows a further sectional perspective view of a part and/or components of the transfer device of FIG. 19;

FIG. 33 shows a schematic view of a part and/or components of the transfer device of FIG. 19;

FIG. 34 shows a schematic top view of a system consisting of two process units according to an exemplary embodiment of the present invention;

FIG. 35 shows a schematic lateral view of a part and/or components of a transfer device for enabling an in particular aseptic transition between a first process unit and a second process unit according to a further exemplary embodiment of the present invention;

FIG. 36 shows a schematic lateral view of a part and/or components of a transfer device for enabling an in particular aseptic transition between a first process unit and a second process unit according to a further exemplary embodiment of the present invention;

FIG. 37 shows a schematic top view of a part and/or components of a transfer device for enabling an in particular aseptic transition between a first process unit and a second process unit according to a further exemplary embodiment of the present invention;

FIG. 38 shows a schematic lateral view of the part and/or components of FIG. 37;

FIG. 39 shows a schematic top view of a part and/or components of a transfer device for enabling an in particular aseptic transition between a first process unit and a second process unit according to a further exemplary embodiment of the present invention;

FIG. 40 shows a schematic lateral view of the part and/or components of FIG. 39;

FIG. 41 shows a schematic top view of a part and/or components of a transfer device for enabling an in particular aseptic transition between a first process unit and a second process unit according to a further exemplary embodiment of the present invention;

FIG. 42 shows a schematic lateral view of the part and/or components of FIG. 41;

FIG. 43 shows a schematic top view of a part and/or components of a transfer device for enabling an in particular aseptic transition between a first process unit and a second process unit according to a further exemplary embodiment of the present invention;

FIG. 44 shows a schematic lateral view of the part and/or components of FIG. 43;

FIG. 45 shows a schematic top view of a part and/or components of a transfer device for enabling an in particular aseptic transition between a first process unit and a second process unit according to a further exemplary embodiment of the present invention;

FIG. 46 shows a schematic lateral view of the part and/or components of FIG. 45;

FIG. 47 shows a schematic lateral view of a part and/or components of a transfer device for enabling an in particular aseptic transition between a first process unit and a second process unit according to a further exemplary embodiment of the present invention;

FIG. 48 shows a schematic top view of the part and/or components of FIG. 47;

FIG. 49 shows a schematic lateral sectional view of a part and/or components of a transfer device for enabling an in particular aseptic transition between a first process unit and a second process unit according to a further exemplary embodiment of the present invention;

FIG. 50 shows a further schematic lateral sectional view of the part and/or components of FIG. 49;

FIG. 51 shows a schematic lateral view of a part and/or components of a transfer device for enabling an in particular aseptic transition between a first process unit and a second process unit according to a further exemplary embodiment of the present invention;

FIG. 52 shows a schematic lateral view of a part and/or components of a transfer device for enabling an in particular aseptic transition between a first process unit and a second process unit according to a further exemplary embodiment of the present invention;

FIG. 53 shows a schematic lateral sectional view of a part and/or components of a transfer device for enabling an in particular aseptic transition between a first process unit and a second process unit according to a further exemplary embodiment of the present invention; and

FIG. 54 shows a further schematic lateral sectional view of the part and/or components of FIG. 53.

Identical or functionally equivalent elements are provided with the same reference signs in all of the figures.

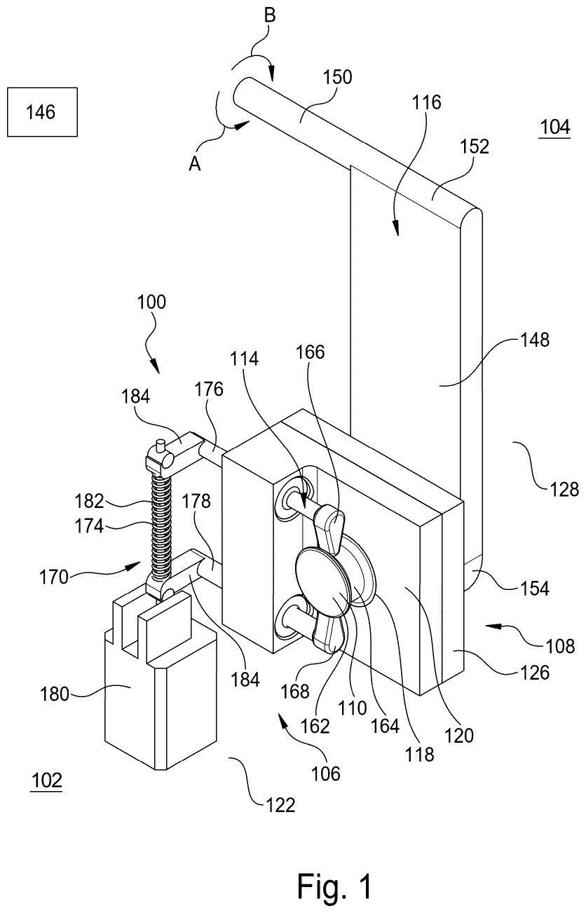

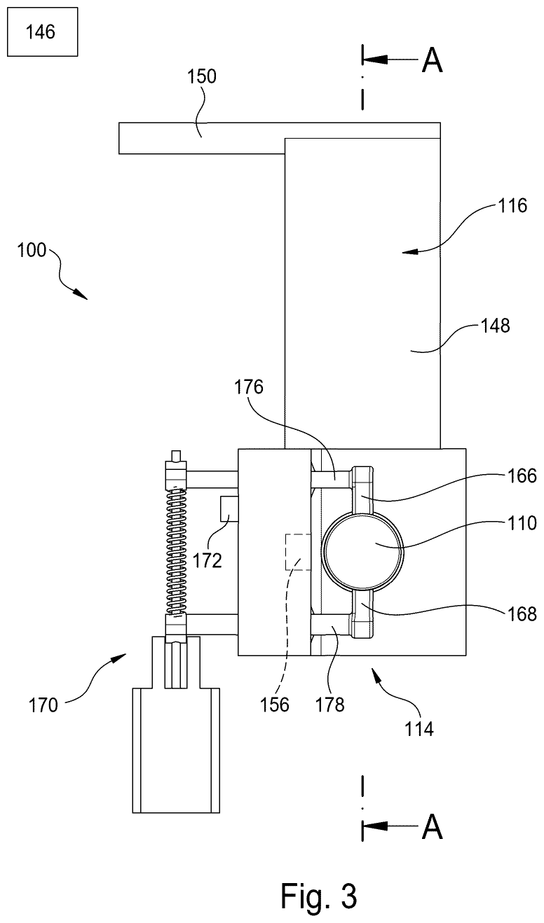

With reference to FIGS. 1 to 54, the embodiments of the present invention described therein are described in connection with objects designed as process units. This use of the present invention can be particularly advantageous, for example.

### DETAILED DESCRIPTION OF THE DRAWINGS

In FIGS. 1 to 5, a transfer device 100 for enabling an in particular aseptic transition between an, e.g. first, process unit 102 and another, e.g. second, process unit 104 according to an exemplary embodiment is schematically illustrated.

The transfer device 100 consequently serves to enable an, or the, in particular aseptic transition between the first process unit 102 and the second process unit 104.

The transfer device 100 is able to be installed at least partially in the first process unit 102 and at least partially in the second process unit 104.

In particular, a first part 106 of the transfer device 100 is able to be installed in the first process unit 102 and a second part 108 of the transfer device 100 is able to be installed in the second process unit 104.

Referring to FIGS. 1 to 5, the transfer device 100 has a first door element 110, a second door element 112, a locking apparatus 114 and an opener apparatus 116.

The first door element 110 is able to be disposed, or in the installed state is disposed, on the first process unit 102.

The first door element 110 is thus assignable or assigned to the first part 106 of the transfer device 100.

The first door element 110 serves to open and close an access opening 118 of the first process unit 102.

The access opening 118 of the first process unit 102 is formed in a wall portion 120 of the first process unit 102 and enables access to an interior 122 of the first process unit 102.

In other words, a first door element 110 which in the installed state is disposed on the first process unit 102 is provided for opening and closing an access opening 118, formed in a wall portion 120 of the first process unit 102 and leading to an interior 122 of the first process unit 102.

The second door element 112 is able to be disposed, or in the installed state is disposed, on the second process unit 104.

The second door element 112 is thus assignable or assigned to the second part 108 of the transfer device 100.

The second door element 112 serves to open and close an access opening 124 of the second process unit 104.

The access opening 124 of the second process unit 104 is formed in a wall portion 126 of the second process unit 104 and enables access to an interior 128 of the second process unit 104.

In other words, a second door element 112 which in the installed state is disposed on the second process unit 104 is provided for opening and closing an access opening 124 formed in a wall portion 126 of the second process unit 104 and leading to an interior 128 of the second process unit 104.

By means of the locking apparatus 114, the first door element 110 is able to be locked in a closed state.

In other words, the locking apparatus 114 serves to lock the first door element 110 in a closed state.

By means of the locking apparatus 114, the first door element 110 is able to be released for opening the latter.

In other words, the locking apparatus 114 serves to release the first door element 110 for opening the latter.

The locking apparatus 114 is able to be disposed, or in the installed state is disposed, in and/or on the first process unit 102.

The locking apparatus 114 is thus assignable or assigned to the first part 106 of the transfer device 100.

By means of the opener apparatus 116, at least one of the door elements 110, 112 is able to be opened and/or closed.

In other words, the opener apparatus 116 serves to open and close at least one of the door elements 110, 112.

The opener apparatus 116 is able to be disposed, or in the installed state is disposed, in and/or on the second process unit 104.

The opener apparatus 116 is thus assignable or assigned to the second part 108 of the transfer device 100.

Furthermore, the first door element 110 and the second door element 112 are able to be brought to engage with one another in order to be removable conjointly from the access openings 118, 124 by means of the opener apparatus 116.

In the engaged state of the door elements 110, 112 and, when the first door element 110 is released by means of the locking apparatus 114, the first door element 110 and the second door element 112 are removable conjointly from the access openings 118, 124 by means of the opener apparatus 116.

As a result, said in particular aseptic transition between the first process unit 102 and the second process unit 104 can be enabled and/or is implemented.

In the present exemplary embodiment, the first process unit 102 is designed as a treatment box, e.g. as a patient box, in particular for receiving and/or transporting and/or treating a treatment object.

Furthermore, in the present exemplary embodiment, the second process unit 104 is designed as a tool box, in particular for treating the treatment object, which in particular can comprise a tool unit, in particular for treating the treatment object, and/or a sensor unit, in particular for analyzing the treatment object.

It can be conceivable that the first and/or the second process unit 102, 104 are designed as a supply box, in particular for providing a material, e.g. a consumable material, and/or a substance, which can be required for treating a, or the, treatment object.

The in particular aseptic transition between the process units 102, 104 serves to supply any item, for example a treatment object, a tool unit, a sensor unit, a material and/or a substance, to one process unit 102 from the other process unit 104, in particular to supply said item temporarily and/or permanently.

The in particular aseptic transition between the process units 102, 104 is able to be used bidirectionally and/or unidirectionally, for example for exchanging any item between the process units 102, 104.

In other words, the in particular aseptic transition between the process units 102, 104 is a bidirectional and/or a unidirectional transition.

In the present exemplary embodiment, a treatment object can comprise, for example, at least one of the following:
an educt, in particular a cell-containing material, for example a cell-containing primary material, and/or a biological-pharmaceutical material and/or a biological-pharmaceutical workpiece,
an intermediate product, in particular a cell-containing intermediate product and/or a biological-pharmaceutical intermediate product,
a product, for example the product to be manufactured, in particular a biological-pharmaceutical product, for example the biological-pharmaceutical product to be manufactured.

It is to be understood that the previous enumeration is exemplary and not exhaustive. Alternatively or additionally, it can be provided that the treatment object comprises one or a plurality of, and/or a combination of, the enumeration elements described above.

In the present exemplary embodiment, a respective process unit 102, 104 is movable and/or transportable and/or displaceable, in particular in an automated manner, relative to its environment.

For example, it is conceivable that a respective process unit 102, 104 can comprise an in particular freely moving transport vehicle or can be designed as such, and/or can be able to be coupled to an in particular freely moving transport vehicle.

For example, it is further conceivable that a respective process unit 102, 104 defines a process cell, in particular an aseptic process cell. In other words, a respective process unit 102, 104 can be a process cell, in particular an aseptic process cell.

Each process unit 102, 104 comprises an in particular lockable interior space, wherein said interior space corresponds to the respective interior 122, 128 of the first and the second process unit 102, 104.

Furthermore, each process unit 102, 104 comprises a shell wall which encloses the respective interior space or the respective interior 122, 128 and thus defines the latter.

Said shell wall comprises the respective wall portion 120, 126 of the first and the second process unit 102, 104 and/or is partially formed by the latter.

Due to the interior space or the interior 122, 128 of a respective process unit 102, 104, an in particular aseptic production area is able to be defined, or is defined and/or formed. In other words, it can be provided that the interior space or the interior 122, 128 is an in particular aseptic production area.

The respective access openings 118, 124 of the first and the second process unit 102, 104 enable a respective access to a respective, in particular aseptic, production area of the first and the second process unit 102, 104.

In other words, the first door element 110 which in the installed state is disposed on the first process unit 102 serves to open and close the access opening 118 to an in particular aseptic production area in the interior 122 of the first process unit 102.

In other words, the second door element 112 which in the installed state is disposed on the second process unit 104 serves to open and close the access opening 124 to an in particular aseptic production area in the interior 128 of the second process unit 104.

The first door element 110 and the second door element 112 have in each case a shell surface which is directed outward relative to the respective process unit 102, 104 and which has a hygienically compliant design embodiment, in particular a hygienic design.

Hygienic design can be understood in particular to mean a design of parts, components and/or production systems that is suitable for cleaning and/or sterilization.

For the hygienically compliant design, the first door element 110 and the second door element 112 have no undercuts on the outside and in particular on the shell surface.

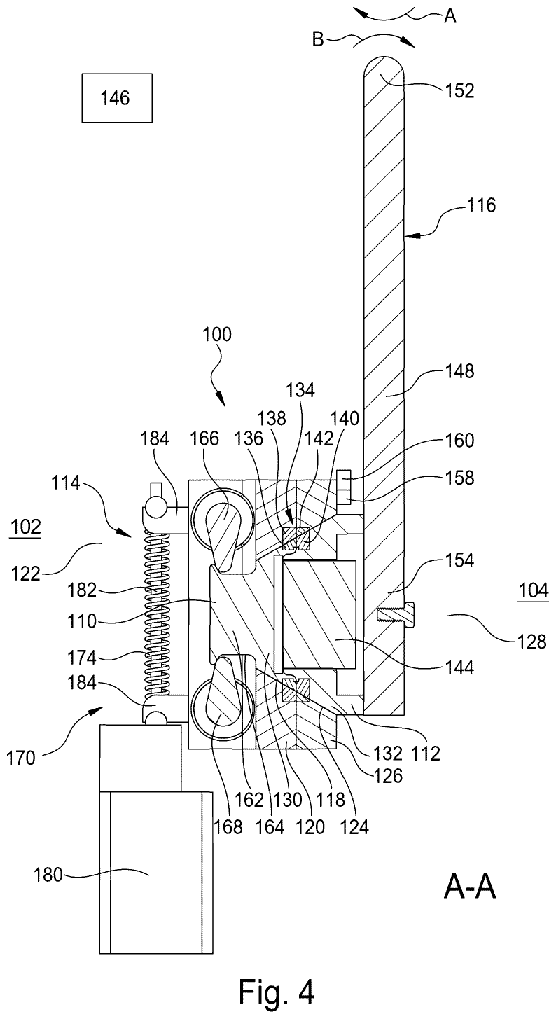

As can be seen in particular in FIG. 4, the first door element 110 comprises a truncated conical portion 130.

The truncated conical portion 130 of the first door element 110 tapers in a direction which in the closed state of the first door element 110 is directed toward the interior 122 of the first process unit 102.

A circumferential surface of said portion 130 of the first door element 110 in the closed state of the first door element 110 rests on an inner circumferential surface of the access opening 118 of the first process unit 102.

Further, it is particularly apparent in FIG. 4 that the second door element 112 comprises a truncated conical portion 132.

The truncated conical portion 132 of the second door element 112 tapers in a direction which in the closed state of the second door element 112 is directed outward relative to the second process unit 104.

A circumferential surface of said portion 132 of the second door element 112 in the closed state of the second door element 104 rests on an inner circumferential surface of the access opening 124 of the second process unit 104.

Referring to FIG. 4, the transfer device 100 has a sealing apparatus 134 which is formed from and/or comprises a plurality of sealing sub-elements 136, 138, 140, 142.

In the closed state of the first door element 110, the first door element 110 and an inner circumferential surface of the access opening 118 of the first process unit 110 are able to be sealed, or are sealed, in relation to the outside by means of the sealing apparatus 134.

Furthermore, in the closed state of the second door element 112, the second door element 112 and an inner circumferential surface of the access opening 124 of the second process unit 104 are able to be sealed, or are sealed, in relation to the outside by means of the sealing apparatus 134.

Furthermore, in the engaged state of the first and the second door element 110, 112, respective surfaces of both door elements 110, 112, which in the respective closed state of the corresponding door elements 110, 112 are directed outward relative to the corresponding process unit 102, 104, are able to be sealed, or are sealed, in relation to the outside by means of the sealing apparatus 134.

Furthermore, in a state in which the respective wall portions 120, 126 of the process units 102, 104 rest on one another to bring the first and the second door element 110, 112 to engage with one another (cf. FIGS. 1, 2, 4, and 5), respective opening regions of both access openings 118, 124 of both process units 102, 104 can be able to be sealed, or are sealed, in relation to the outside by means of the sealing apparatus 134.

The sealing apparatus 134 and in particular each sealing sub-element 136, 138, 140, 142 have a closed and circular contour.

As a result of this closed and circular contour, the above-mentioned components are consequently able to be sealed, or are sealed, radially and axially in relation to the outside.

As can be seen in FIG. 4, the sealing apparatus in the present exemplary embodiment comprises four sealing sub-elements 136, 138, 140, 142.

A first sealing sub-element 136 is disposed on a peripheral region of the first door element 110.

This peripheral region of the first door element 110 is, in the closed state of the first door element 110, adjacent to the inner circumferential surface of the access opening 118 of the first process unit 102 and directed outward relative to the first process unit 102.

A second sealing sub-element 138 is able to be disposed, or is disposed, on an edge region of the access opening 118 of the first process unit 102.

This edge region of the access opening 118 of the first process unit 102 is, in the closed state of the first door element 110, adjacent to the first door element 110 and in particular to said peripheral region of the first door element 110 and directed outward relative to the first process unit 102.

A third sealing sub-element 140 is disposed on a peripheral region of the second door element 112.

This peripheral region of the second door element 112 is, in the closed state of the second door element 112, adjacent to the inner circumferential surface of the access opening 124 of the second process unit 104 and directed outward relative to the second process unit 104.

A fourth sealing sub-element 142 is able to be disposed, or is disposed, on an edge region of the access opening 124 of the second process unit 104.

This edge region of the access opening 124 of the second process unit 104 is, in the closed state of the second door element 112, adjacent to the second door element 112 and in particular to said peripheral region of the second door element 112 and directed outward relative to the second process unit 104.

The first and the third sealing sub-element 136, 140 in the engaged state of the first and the second door element 110, 112 are disposed directly next to one another (cf. FIG. 4).

By means of the first and the third sealing sub-element 136, 140, the respective surfaces of both door elements 110, 112, which in the respective closed state of the corresponding door elements 110, 112 are directed outward relative to the corresponding process unit 102, 104 and are bordered by the respectively assigned first and third sealing sub-element 136, 140, are able to be sealed, or are sealed, in relation to the outside.

This can be understood to mean, in particular, that a respective surface of the mutually engaged door elements 110, 112, which in the engaged state of the door elements 110, 112 face one another and/or on which the door elements 110, 112 rest on one another, can be able to be sealed, or can be sealed, in relation to the outside by means of the first and third sealing sub-element 136, 140.

The second and the fourth sealing sub-element 138, 142 are able to be disposed, or are disposed, directly next to one another, for example in a, or the, state in which the respective wall portions 120, 126 of the process units 102, 104 rest on one another for a, or the, bringing into engagement of the first and the second door element 110, 112 with one another (cf. FIGS. 1, 2, 4 and 5).

By means of the second and fourth sealing sub-element 138, 142, respective opening regions of both access openings 118, 124 of both process units 102, 104 are able to be sealed, or are sealed, in relation to the outside.

In the present exemplary embodiment, a respective sealing sub-element 136, 138, 140, 142 is able to be manufactured, or is manufactured, from a plastics material. For example, a respective sealing sub-element 136, 138, 140, 142 can comprise and/or be at least partially formed from polytetrafluoroethylene (PTFE) and/or polyvinyl chloride (PVC) and/or silicone. This can advantageously enable a positive autoclaving capability.

Referring to FIG. 4, the second door element 112 comprises a coupling element 144.

The coupling element 144 is provided in and/or on the second door element 112 and forms at least a part of the second door element 112.

By means of the coupling element 144, the first door element 110 is able to be brought to engage with the second door element 112 and able to be held thereon.

In the present exemplary embodiment, the coupling element 114 is configured to bring the first door element 110 to engage with the second door element 112 in a magnet-based manner and to hold it thereon in a magnet-based manner. However, it is also conceivable that the coupling element 144 can be configured so as to be activatable in a force-fitting, form-fitting and/or vacuum-based manner, or is able to be operable in such a way.

As can be seen in FIG. 4, the coupling element 144 forms a part of the second door element 112 directed outward relative to the second process unit 104.

This part of the second door element 112 has a flush transition to the remaining part or rest of the second door element 112, the latter being directed outward relative to the second process unit 104.

The coupling element 144 is presently disposed so as to be substantially centric in the truncated conical portion 132 of the second door element 112.

In the present exemplary embodiment, the coupling element 144 is designed as a solenoid.

The first door element 110 is correspondingly partially or completely formed as a magnetic counter-element. For this purpose, the first door element 110 is able to be manufactured, or is manufactured, from a magnetic or magnetizable material, for example.

The coupling element 144, which is designed as a solenoid, is actuatable by means of a control unit 146 in order to bring the first door element 110 to engage with the second door element 112 and to hold it thereon.

The control unit 146 can be comprised by the transfer device 100 and/or be assignable, or assigned, to the transfer device 100. For example, a control unit 146 which is assignable, or assigned, to the transfer device 100 can be comprised by the first and/or the second process unit 102, 104.

As can furthermore be seen from FIGS. 1 to 5, the opener apparatus 116 has a pivotable lever element 148 and a center of rotation 150.

The lever element 148 is, in the installed state, able to be pivotably fixed, or is pivotably fixed, in the second process unit 104 on a first end region 152 of the lever element 148.

Presently, the lever element 148 is fixed in the second process unit 104 in order to be able to pivot the second door element 112 toward a lateral surface of the interior 128 of the second process unit 104.

Furthermore, the second door element 112 is fastened to a second end region 154 of the lever element 148.

The pivotable lever element 148 and in particular the second end region 154 of the lever element 148 are rigidly connected to the second door element 112.

By means of the lever element 148, the second door element 112, or the mutually engaged door elements 110, 112 conjointly, are movable, in particular in a pivoting manner, in the interior 128 of the second process unit 104.

In the present exemplary embodiment, this is a purely pivoting movement. However, it is also conceivable that a tilting and/or a linear movement may exist.

As a result, the second door element 112, or the mutually engaged door elements 110, 112 conjointly, are removable from the access openings 118, 124.

In the present exemplary embodiment, the lever element 148 is disposed by way of the center of rotation 150 in the interior 128 of the second process unit 104.

The lever element 148 is rotatable and pivotable by way of the center of rotation 150.

Presently, the center of rotation 150 is formed integrally with the first end region 152 of the lever element 148.

The opening and closing of at least one of the door elements 110, 112—presently the second door element 112 or the mutually engaged door elements 110, 112—is able to be initiated by means of a corresponding rotating and/or pivoting movement.

It is understood that this rotating and/or pivoting movement is able to take place, or takes place, about the center of rotation 150.

Specifically, opening of at least one of the door elements 110, 112—presently the second door element 112 or the mutually engaged door elements 110, 112—takes place in a direction of rotation which is indicated by A in the figures. In other words, this is an opening rotation direction A.

Specifically, closing of at least one of the door elements 110, 112—presently the second door element 112 or the mutually engaged door elements 110, 112—takes place in a direction of rotation which is indicated by B in the figures. In other words, this is a closing rotation direction B.

Referring to FIGS. 1 to 5, the center of rotation 150 is advantageously disposed in the installed state at a spacing from the access opening 124 of the second process unit 104.

Said spacing extends in particular between the center of rotation 150 and a center, in particular center point, of the access opening 124 of the second process unit 104.

This spacing is greater than, or greater than or equal to, a widest opening dimension of the access opening 124 of the second process unit 104 and specifically than an opening diameter of the access opening 124 of the second process unit 104.

For example, the spacing can be at least 100%, in particular at least 150%, preferably at least 200%, further preferably at least 250%, in particular preferably at least 300%, greater than, or greater than or equal to, the widest opening dimension of the access opening 124 of the second process unit 104, in particular than the opening diameter of the access opening 124 of the second process unit 104.

It is understood that the opener apparatus 116 and in particular the lever element 148 for said opening and closing is able to be motorized, or is motorized.

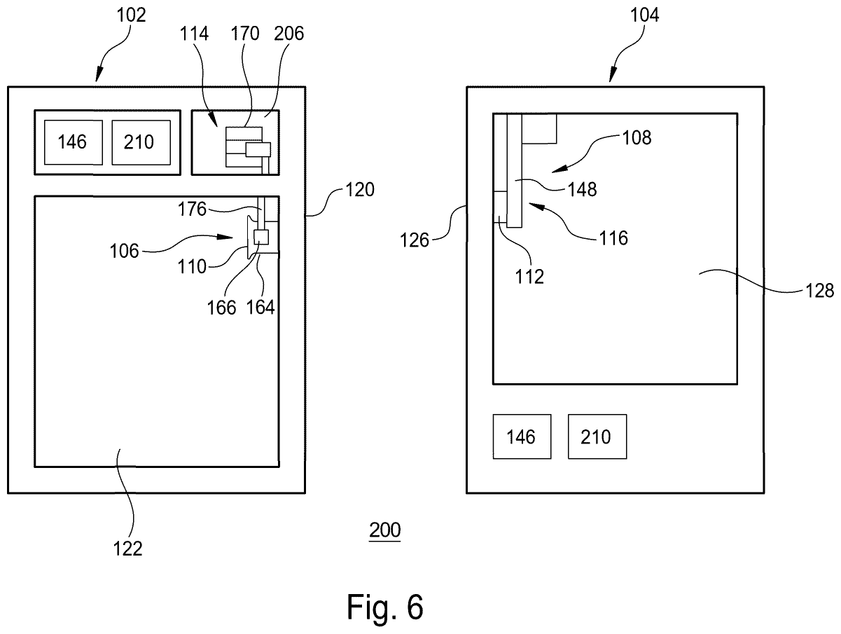

A corresponding motor apparatus is provided for this purpose (shown by way of example with the reference sign 208 in FIG. 6).

This motor apparatus is able to be disposed, or is disposed, in and/or on the second process unit 104.

The motor apparatus is advantageously comprised by the transfer device 100 and in particular the opener apparatus 116.

The opener apparatus 116 and in particular the lever element 148 are activatable, in particular in an automated manner, for said opening and closing by means of the motor apparatus.

The motor apparatus is actuatable by means of the control unit 146.

Furthermore, the transfer device 100 comprises sensor units 156, 158 for detecting an open state of the first door element 110 and for detecting an open state of the second door element 112.

Specifically, a first sensor unit 156 serves for detecting an open state of the first door element 110, and a second sensor unit 158 serves for detecting an open state of the second door element 112.

These sensor units 156, 158 are furthermore connected, in particular for signaling, to the control unit 146.

This serves in particular for detecting the open state of the first door element 110 and for detecting the open state of the second door element 112 by means of the control unit 146, and furthermore for determining whether transitioning between the first and the second process unit 102, 104 is enabled.

In the present exemplary embodiment, the open state of the first door element 110 and the open state of the second door element 112 are optically detectable by the sensor units 156, 158. It is understood that this is purely exemplary and other sensor configurations are conceivable.

Furthermore, the transfer device 100 comprises a third sensor unit 160.

By means of the third sensor unit 160, it is detectable whether the first door element 110 and the second door element 112 are engaged with one another.

For example, a change in a magnetic field in and/or on the second process unit 104 is detectable by means of the third sensor unit 160.

This change can be caused by the opener apparatus 116 when opening and/or closing.

As a result, it is detectable whether the first door element 110 and the second door element 112 are engaged with one another.

It is understood that this sensor configuration is purely exemplary and other sensor configurations are conceivable.

The third sensor unit 160 is also connected, in particular for signaling, to the control unit 146.

This serves in particular to determine whether the first door element 110 and the second door element 112 are engaged with one another.

As already described, the first door element 110 has a truncated conical portion 130.

Referring to FIGS. 1 to 5, the first door element 110 further comprises a holding portion 162.

The holding portion 162 is disposed on the truncated conical portion 130 of the first door element 110 and extends away from the latter.

In particular, this holding portion in the locked state of the first door element 110 extends into the interior 122 of the first process unit 102.

The holding portion 162 is of a substantially cylindrical shape.

A holding contour 164 is formed on and/or in said holding portion 162.

The holding contour 164 in the closed state (of the first door element 110) is disposed internally relative to the first process unit 102, in particular disposed in the interior 122 of the first process unit 102 and facing the latter.

The holding contour 164 is designed in the form of a depression, for example an encircling groove, in a shell surface of the holding portion 162.

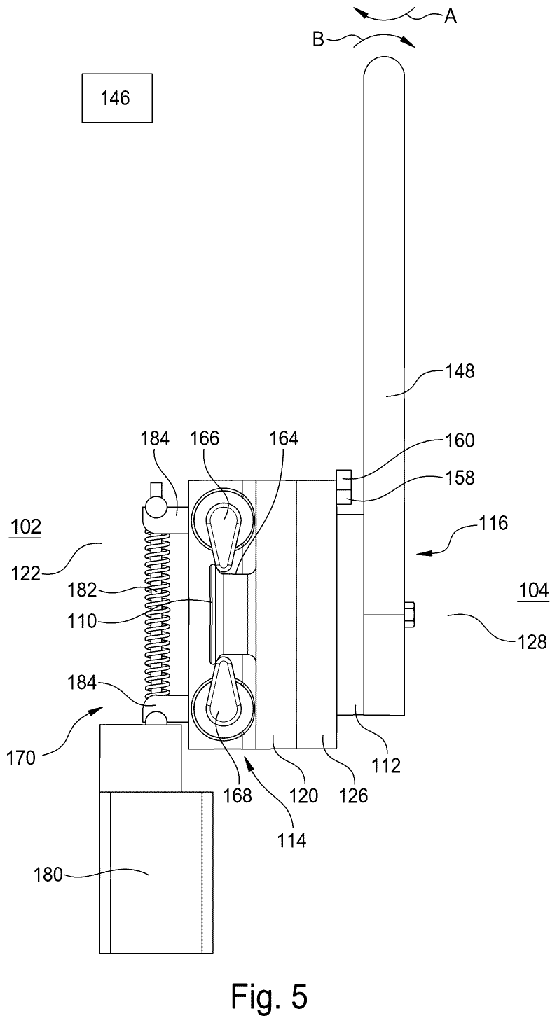

The holding portion 162 is therefore substantially mushroom-shaped (cf., e.g., FIGS. 4 and 5).

The holding portion 162 and in particular the holding contour 164 serve to lock the first door element 110.

The locking apparatus 114 comprises a first holding element 166 and a second holding element 168, which likewise serve for locking the first door element 110.

The holding elements 166, 168 in the installed state are disposed in the first process unit 102.

The holding elements 166, 168 are able to be selectively brought to engage with the holding contour 164 for locking the first door element 110.

The holding elements 166, 168 are disposed on opposite sides of the holding contour 164 in order to be able to be selectively brought to engage with the holding contour 164 for locking the first door element 110.

The holding elements 166, 168 are motorized and in particular activatable in an automated manner.

For this purpose, the locking apparatus 114 has a drive apparatus 170.

By means of the drive apparatus 170, the holding elements 166, 168 are movable in order for them to be able to be selectively brought to engage with the holding contour 164.

In particular, each holding element 166, 168 is movable by means of the drive apparatus 170 between a locked position and a release position.

For example, the locked position is shown in FIG. 4.

The locked position corresponds to a state of the holding elements 166, 168 engaged with the holding contour 164.

The release position corresponds to a state of the holding elements 166, 168 disengaged from the holding contour 164.

The drive apparatus 170 in the installed state is disposed in and/or on the first process unit 102.

In particular, the drive apparatus 170 in the installed state is disposed in the interior 122 of the first process unit 102 and on a wall defining the interior 122 of the first process unit 102, in particular the already described shell wall, of the first process unit 102.

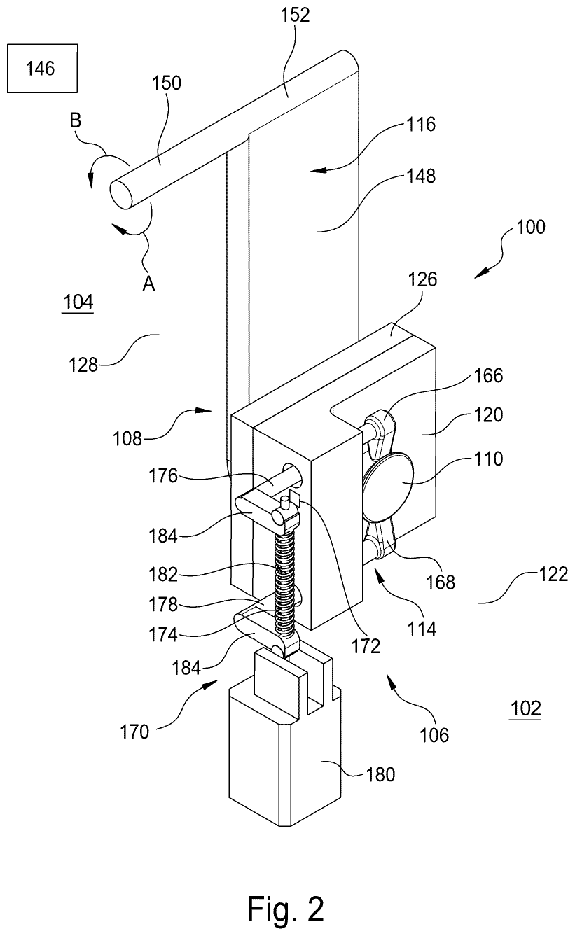

The transfer device 100 furthermore comprises a fourth sensor unit 172 (cf. FIG. 2).

The fourth sensor unit 172 is used to detect a locked state of the first door element 110.

By means of the fourth sensor unit 172, a position of the holding elements 166, 168 relative to the drive apparatus 170 and the holding contour 164 is detectable.

For example, the locked state of the first door element 110 is detectable by the fourth sensor unit 172 by means of a position detection of the holding elements 166, 168 relative to the drive apparatus 170 and the holding contour 164, by way of which the position of the holding element 166, 168 is able to be determined.

It is understood that this sensor configuration is purely exemplary and other sensor configurations are conceivable.

The fourth sensor unit 172 is likewise connected, in particular for signaling, to the control unit 146, in particular for detecting the locked state of the first door element 110.

As can be seen in FIGS. 1 to 5, the drive apparatus 170 comprises an elastic element which is presently designed as a spring element 174.

By means of the spring element 174, the first and the second holding element 166, 168 are able to be preloaded, or are preloaded, in the direction of engagement with the holding contour 164 and in particular in the direction of the locked position.

The drive apparatus 170 is operatively connected to the first holding element 166 by way of a first shaft 176.

The drive apparatus 170 is operatively connected to the second holding element 168 by way of a second shaft 178.

The first and the second holding element 166, 168 are in each case able to be brought to engage with the holding contour 164 and brought back out of the latter by way of the first and the second shaft 176, 178.

This can be understood to mean in particular that the first and second holding element 166, 168 are in each case movable to the locked position and the release position by way of the first and the second shaft 176, 178.

In other words, the drive apparatus 170 is operatively connected to the respective holding elements 166, 168 by way of a respective shaft 176, 178, the holding elements 166, 168 being able to be brought to engage with the holding contour 164 and brought back out of the latter, and in particular being movable into the locked position and the release position, by way of said shafts 176, 178.

For this purpose, the drive apparatus 170 has a motor 180 and a spindle drive 182, for example.

The motor 180 is operatively connected to each shaft 176, 178 and thus to each holding element 166, 168.

By means of the motor 180, a force and/or a torque by way of which each holding element 166, 168 is able to be initiated to move is able to be generated.

As a result, each holding element 166, 168 is movable selectively to the locked position or to the release position.

In particular, the spindle drive 182 is able to be driven by way of the motor 180, as a result of which the force and/or the torque is able to be transmitted.

For this purpose, an activating arm 184 is formed on each shaft 176, 178.

Each activating arm 184 is movably connected to the spindle drive 182.

By way of a respective activating arm 184, the respective holding elements 166, 168 are able to be initiated to move in order to move the holding elements 166, 168 selectively to the locked position or to the release position.

Preferably, a rotating movement of the spindle drive 182 is able to be transmitted in the form of a pivoting movement to the respective shaft 176, 178 by way of the connection to the activating arms 184 of the shafts 176, 178, said pivoting movement being able to be transmitted to the holding elements 166, 168 by way of the shafts 176, 178.

This can be understood to mean in particular that the drive apparatus 170 comprises a, or the, motor 180 and a spindle drive 182 driven by means of the motor 180, and formed on the respective shafts 176, 178 is in each case an activating arm 184, the latter being movably connected to the spindle drive 182, wherein a rotating movement of the spindle drive 182 is able to be transmitted in the form of a pivoting movement to the respective shaft 176, 178 by way of the connection to the respective activating arm 184 of the respective shaft 176, 178, said pivoting movement being able to be transmitted to the respective holding element 166, 168 by way of the respective shaft 176, 178.

Here, the two holding elements 166, 168 can be brought to engage with the holding contour 164 and brought back out of the latter, and in particular are movable into the locked position and into the release position, by means of a mutually opposing and in particular synchronous pivoting movement.

This can be understood to mean in particular that when the activating arms 184 are moved toward one another by way of the spindle drive 182, the holding elements 166, 168 are moved out of the holding contour 164 and, when the activating arms 184 are moved away from one another by way of the spindle drive 182, the holding elements 166, 168 are moved into the holding contour 164.

The drive apparatus 144 and in particular the motor 180 are actuatable by means of the control unit 146.

This serves in particular to be able to initiate each holding element 166, 168 to move, in particular to be able to move to the locked position and the release position.

As a result, locking of the first door element 110 can take place in a motorized, in particular automated, manner.

As can be seen in the figures, the drive apparatus 170 in the installed state is disposed within the first process unit 102.

It is conceivable here that the drive apparatus 170 is partially or completely disposed in a separate interior space of the first process unit 102, which differs from the interior 122 of the first process unit 102 (cf. FIG. 6).

It is further conceivable here that the drive apparatus 170 in the installed state is disposed within the first process unit 102 and is designed to be at least partially or completely autoclavable. In this respect, the drive apparatus 170 comprises an in particular sealed housing which is partially or completely insulated, in particular thermally, relative to its environment.

It is moreover understood that the control unit 146 and/or its signaling connections is/are able to be selectively disconnected and/or de-energized for autoclaving of the transfer device 100 and in particular for autoclaving of the respective interior 122, 128 of the first and the second process unit 102, 104.

Referring to FIGS. 1 to 5, a transfer device 100 is now provided, by means of which any item, e.g. a treatment object, a tool unit, a sensor unit, a material and/or a substance is able to be exchanged aseptically between two process units 102, 104.

In particular, the simple and robust configuration of the transfer device 100 allows a capability for easy cleaning and/or sterilizing, in particular autoclaving.

Moreover, the transfer device 100 is in particular operable in a fully automated manner, so that it can facilitate a large-scale application and/or an industrial production of individual and/or very small batches.

A system 200 according to the invention, consisting of at least two process units 102, 104, 202, 204, will be described in more detail hereunder.

It should be understood that all structural and/or functional features which are related to the transfer device 100 according to the invention described above and/or below and/or the process units 102, 104 according to the invention described above and/or below can also be contained in said system 200 either alone or in combination and the associated properties, embodiments and/or advantages can likewise be accordingly included and achieved.

The system 200 is formed from, and/or comprises, at least two process units 102, 104.

Referring to FIG. 6, a system 200 consisting of two process units 102, 104, specifically the first and the second process unit 102, 104, is provided.

The two process units 102, 104 comprise a transfer device 100 according to the above and/or following embodiments.

Each process unit 102, 104 serves to manufacture a product, in particular a biological-pharmaceutical product, as already been described at the outset.

In the present exemplary embodiment, the first process unit 102 is formed as a treatment box, e.g. as a patient box, in particular for receiving and/or transporting and/or treating a treatment object.

Furthermore, in the present exemplary embodiment, the second process unit 104 is formed as a tool box in particular for treating the treatment object, which in particular can comprise a tool unit in particular for treating the object and/or a sensor unit in particular for analyzing the treatment object.

It can be conceivable that the first and/or the second process unit 102, 104 are designed as a supply box, in particular for providing a material, e.g. a consumable material, and/or a substance, which can be required for treating a, or the, treatment object.

The first part 106 of the transfer device 100 is installed in the first process unit 102.

The second part 108 of the transfer device 100 is installed in the second process unit 104.

As can be seen in FIG. 6, the drive apparatus 170 is disposed in a separate interior space 206 of the first process unit 102, which differs from the interior 122 of the first process unit 102 (cf. FIG. 6).

A corresponding operative connection between the drive apparatus 170 and the holding elements 166, 168 is implemented by way of the shafts 176, 178.

In other words, the shafts 176, 178 presently serve as a coupling component to connect the respective portions of the transfer device 100 to one another. This can, for example, facilitate autoclavability.

The motor apparatus 208 described in the context of FIGS. 1 to 5 is furthermore schematically shown in FIG. 6.

In the present exemplary embodiment, the transfer device 100 is actuatable by way of the control unit 146 of the first and the second process unit 102, 104.

Furthermore, the transfer device 100 is supplied with power by way of an energy storage device 210 of the first and the second process unit 102, 104.

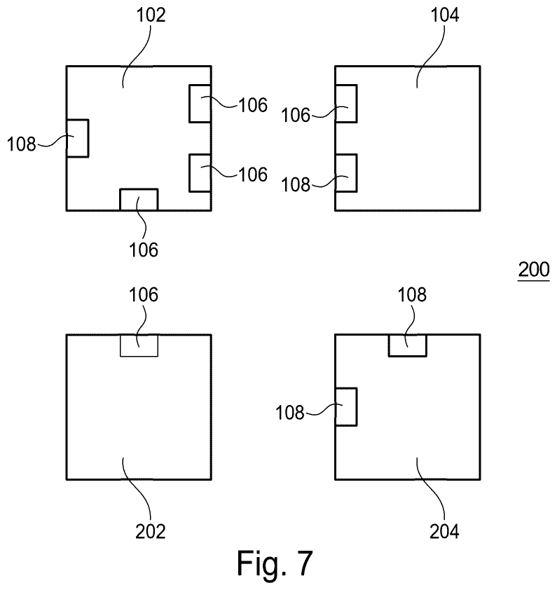

Referring to FIG. 7, a system consisting of four process units 102, 104, 202, 204, specifically the first and the second process unit 102, 104 and a third process unit 202 and a fourth process unit 204, is provided.

Presently, one or a plurality of first and/or one or a plurality of second parts 106, 108 of a transfer device 100 according to the above and/or following embodiments are provided in each process unit 102, 104, 202, 204.

The transfer device 100 is able to be formed from at least one first part 106 and at least one second part 108.

At least one first part 106 and at least one second part 108 of the transfer device 100 are provided in mutually different process units 102, 104, 202, 204.

As already described, a first part 106 can presently be understood to be in particular that part of the transfer device 100 that is assigned to the first door element 110 and/or connected thereto, or that is able to be disposed, or is disposed, in and/or on the first process unit 102.

Furthermore, as has already been described, a second part 108 can presently be understood to be in particular that part of the transfer device 100 that is assigned to the second door element 112 and/or is connected thereto, or that is able to be disposed, or is disposed, in and/or on the second process unit 102.

As can be seen in FIG. 7, process units 102, 104, 202, 204 are provided, in which one or a plurality of first and/or one or a plurality of second parts 106, 108 of the transfer device 100 are provided, and which are able to be connected by means of the transfer device 100 to a further process unit 102, 104, 202, 204, in which at least one first and/or one second part 106, 108 of the transfer device 100 are/is provided, said transfer device being able to be formed from at least one first and at least one second part 106, 108.

The first and/or second parts 106, 108 are each configured on identical sides and/or on different sides of the process units 102, 104, 202, 204.

As a result, an in particular aseptic transition between these process units 102, 104, 202, 204 can be enabled, for example for introducing one or a plurality of tool units and/or one or a plurality of sensor units and/or consumables and/or consumable materials.

In particular, these process units 102, 104, 202, 204 can be connected to one another, in particular aseptically, in a flexible manner and/or according to requirements.

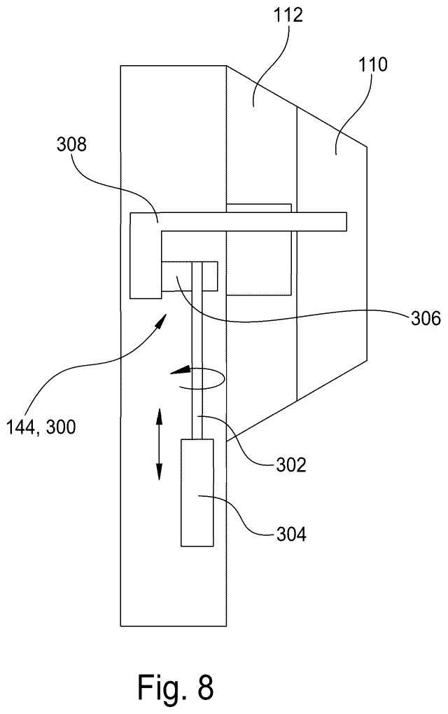

Respective parts and/or components of a transfer device 100 are in each case schematically illustrated in FIGS. 8 to 18, so as to enable an in particular aseptic transition between a first process unit 102 and a second process unit 104 according to respective further exemplary embodiments of the present invention.

It should be understood that all structural and/or functional features described in the context of the transfer device 100 of FIGS. 1 to 7 and/or the process units 102, 104 of FIGS. 1 to 7 are also able to be included in the embodiments of FIGS. 8 to 18, either alone or in combination, and the associated properties, embodiments and/or advantages can likewise be correspondingly included and achieved. It is also understood that this also applies vice versa.

In the following, since the transfer device 100 of FIGS. 1 to 7 is substantially identical to the respective transfer devices 100 of FIGS. 8 to 18, only the respective differences are described in the context of FIGS. 8 to 18.

Referring to FIG. 8, shown there is a part of a transfer device 100 according to a further exemplary embodiment, in which the coupling element 144 is configured to bring the first door element 110 to engage with the second door element 112 in particular in a force-fitting and/or form-fitting manner, and in particular to hold it thereon in a force-fitting and/or form-fitting manner.

The coupling element 144 is designed as an eccentric apparatus 300.

The eccentric apparatus 300 comprises a shaft 302 and a motion apparatus 304.

An eccentric element 306 is formed eccentrically on the shaft 302.

By means of the motion apparatus 304, the shaft 302 is movable and in particular movable linearly and rotationally, this being correspondingly schematically indicated by arrows in FIG. 8.

The shaft 302 is provided in and/or on the second door element 112.

The motion apparatus 304 is able to be disposed, or is disposed, in and/or on the second process unit 104.

A counter-element 308 is provided in and/or on the first door element 110.

The counter-element 308 is able to be brought to engage with the shaft 302 and in particular with the eccentric element 306.

The shaft 302 and in particular the eccentric element 306 is linearly positionable relative to the counter-element 308 by means of the motion apparatus 304, and is subsequently rotatable in order to bring the eccentric element 306 and the counter-element 308 to engage with one another and to push the counter-element 308 from the first door element 110 to the second door element 112.

As a result, the first and the second door element 110, 112 are able to be brought to engage with one another and to be held on one another.

The motion apparatus 304 is actuatable by means of the control unit 146 in order to bring the first door element 110 to engage with the second door element 112 and to hold it thereon.

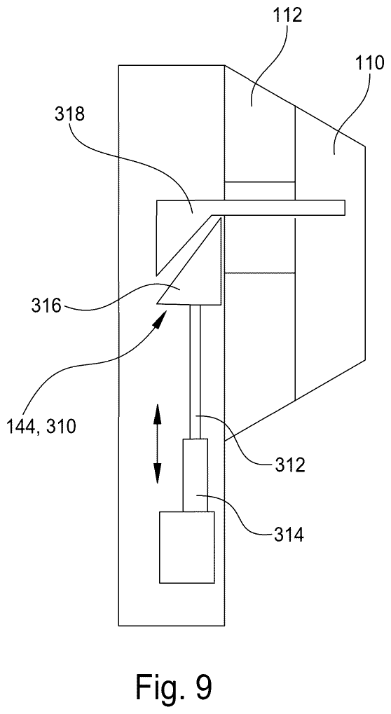

Referring to FIG. 9, shown there is a part of a transfer device 100 according to a further exemplary embodiment, in which the coupling element 144 is configured to bring the first door element 110 to engage with the second door element 112 in particular in a force-fitting and/or form-fitting manner, and in particular to hold it thereon in a force-fitting and/or form-fitting manner.

The coupling element 144 is designed as a wedge apparatus 310.

The wedge apparatus 310 comprises a displacement element 312 and a motion apparatus 314.

A wedge-shaped element 316 is formed on the displacement element 312.

By means of the motion apparatus 314, the displacement element 312 is movable and in particular linearly movable, which is correspondingly schematically indicated by an arrow in FIG. 9.

The displacement element 312 is provided in and/or on the second door element 112.

The motion apparatus 314 is able to be disposed, or is disposed, in and/or on the second process unit 104.

A wedge-shaped counter-element 318 is provided in and/or on the first door element 110.

The counter-element 318 is able to be brought to engage with the displacement element 312 and in particular with the wedge-shaped element 316.

Furthermore, a spring element 320 is provided between the motion apparatus 314 and the displacement element 312, which can initiate the wedge-shaped element 316 to move in the direction of the wedge-shaped counter-element 318 under the action of a spring force.

The displacement element 312 and in particular the wedge-shaped element 316 is linearly movable relative to the counter-element 318 by means of the motion apparatus 314, and subsequently movable along one another under contact so as to push the counter-element 318 from the first door element 110 to the second door element 112.

As a result, the first and the second door element 110, 112 are able to be brought to engage with one another and to be held on one another.

The motion apparatus 314 is actuatable by means of the control unit 146 in order to bring the first door element 110 to engage with the second door element 112 and to hold it thereon.

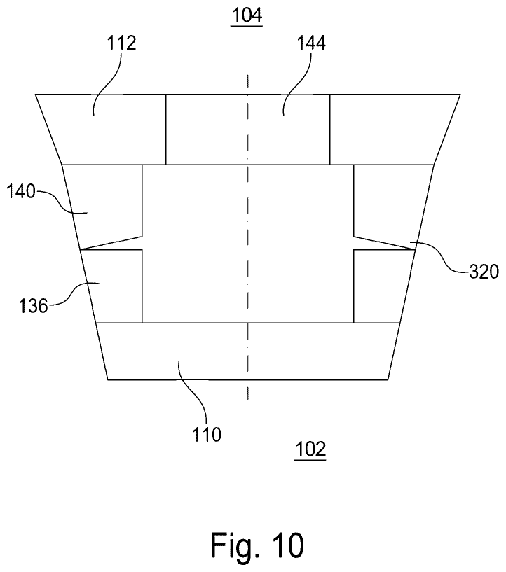

Referring to FIG. 10, shown there in a simplified and enlarged manner is in particular a part of a sealing apparatus 134 of a transfer device 100 according to a further exemplary embodiment, in which at least the first and the third sealing sub-element 136, 140 have a substantially polygonal cross section.

The first and the third sealing sub-element 136, 140 have a conically tapered portion 320 in cross section.

The tapered portion 320 tapers outwardly relative to the respective process unit 102, 104 and in particular the respective door element 110, 112, on which the respective sealing sub-element 136, 140 is disposed.

As a result, a sealing surface is able to be advantageously reduced, whereby high contact forces which can be required for large sealing surfaces can be avoidable.

It is understood that the second and the fourth sealing sub-element 138, 142 can also be configured like the first and the third sealing sub-element 136, 140 described above.

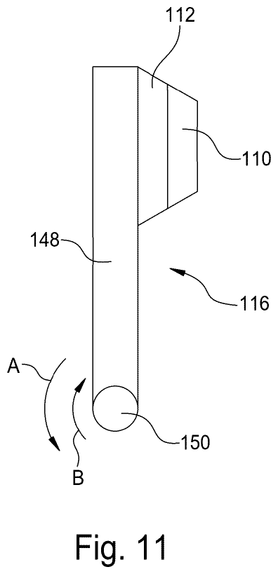

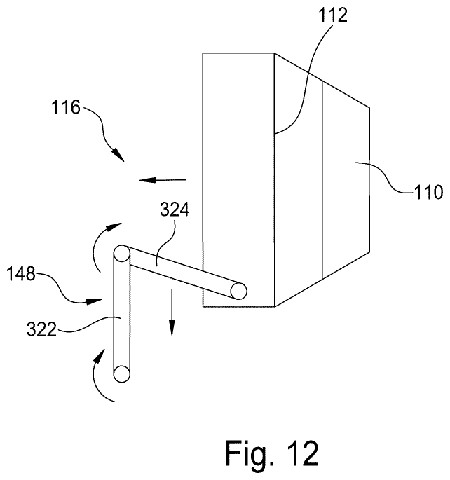

Referring to FIG. 11, the opener apparatus 116 of the transfer device 100 of FIG. 1 is shown there again in simplified form for the purposes of explaining the exemplary embodiments of FIGS. 12 to 14.

Here, the pivotable lever element 144, in particular the second end region 154 of the lever element 144, is rigidly connected to the second door element 112.

By means of the lever element 144, the second door element 112, or the mutually engaged door elements 110, 112 conjointly, is/are movable in a purely pivoting manner into the interior 128 of the second process unit 104 and thus able to be removed from the access openings 118, 124.

Referring to FIG. 12, shown there is a part of a transfer device 100 according to a further exemplary embodiment, in which the opener apparatus 116, and in particular the lever element 148, is designed differently in comparison to FIG. 11.

The pivotable lever element 148 is formed from a plurality of, presently two, lever links 322, 324.

The lever links 322, 324 are movably connected to one another.

Presently, the pivotable lever element 148 is thus formed from a first and a second lever link 322, 324.

In the installed state in the second process unit 104, the first lever link 322 is pivotably fixed on a first end region of the first lever link 322, and a first end region of the second lever link 324 is pivotably fixed on a second end region of the first lever link 322.

The second door element 112 is pivotably fixed on a second end region of the second lever link 324.

By means of the lever links 322, 324, the second door element 112, or the mutually engaged door elements 110, 112 conjointly, is/are movable into the interior 128 of the second process unit 104 and thus able to be removed from the access openings 118, 124 by a movement in a plane.

The movement in a plane is advantageously a two-dimensional linear movement in a plane, which is indicated by straight arrows in FIG. 12.

A first motor (not shown in the figures) is disposed between the second process unit 104 and the first lever link 322, and a second motor (not shown in the figures) is disposed between the first lever link 322 and the second lever link 324.

The first and the second motor can form and/or be comprised by said motor apparatus 208.

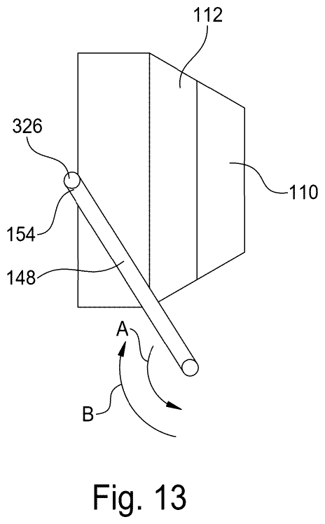

Referring to FIG. 13, shown there is a part of a transfer device 100 according to a further exemplary embodiment, in which the opener apparatus 116, and in particular the lever element 148, is designed differently in comparison to FIG. 11.

The pivotable lever element 148, in particular the second end region 154 of the lever element 148, is movably connected to the second door element 112, in particular by way of an articulation apparatus 326.

By means of the lever element 148, the second door element 112, or the mutually engaged door elements 110, 112 conjointly, is/are movable in a pivoting and linear manner into the interior 128 of the second process unit 104 and thus able to be removed from the access openings 118, 124.

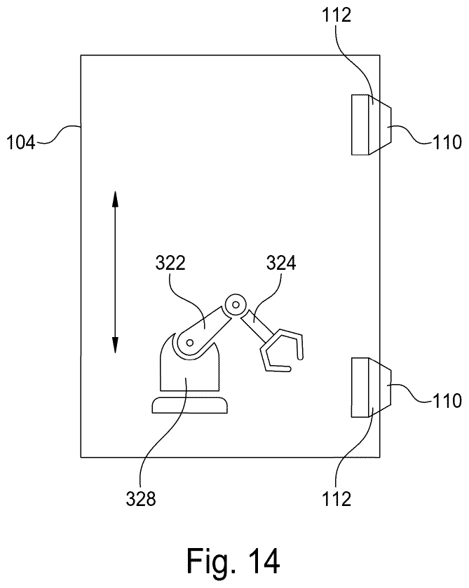

Referring to FIG. 14, shown there is a part of a transfer device 100 according to a further exemplary embodiment, in which the opener apparatus 116, and in particular the lever element 148, is designed differently in comparison to FIG. 11.

In particular, the second process unit 104 is schematically illustrated having a plurality of, presently two, second door elements 112.

The pivotable lever element 148 is formed from two lever links 322, 324 which are movably connected to one another.

The pivotable lever element 148, and thus at least partially the opener apparatus 116, are formed as a part of a handling apparatus 328 and/or are comprised by the latter.

A lever link 324 assigned or assignable to the second door elements 112 is releasably connectable to a respective second door element 112 by way of a gripper actuator mechanism 328 configured on this lever link 324.

The handling apparatus 326 is designed, for example, as a multi-axis robot arm and/or pick-and-place robot.

The handling apparatus 326 in the installed state in the second process unit 104 is rail-guided and displaceable for a wide and in particular larger effective range and in particular handling range (cf. arrow in FIG. 14).

By means of the handling apparatus 326, a respective second door element 112, or the mutually engaged door elements 110, 112 conjointly, is/are movable into the interior 128 of the second process unit 104 and thus able to be removed from respective access openings 118, 124.

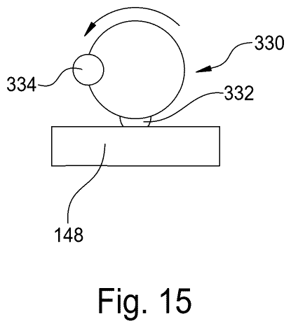

Referring to FIGS. 15 to 18, a part of a transfer device 100 according to a respective further exemplary embodiment is in each case shown therein, in which a further locking apparatus 330 is in each case provided for locking the second door element 112 in a closed state and for releasing the second door element 112 for opening the latter.

The further locking apparatus 330 in the installed state is disposed in and/or on the second process unit 104.

The first door element 110 and the second door element 112 are able to be brought to engage with one another and, in the engaged state, when the first door element 110 is released by means of the locking apparatus 114, and the second door element 112 is released by means of the further locking apparatus 330, are able to be removed conjointly from the access openings 118, 124 by means of the opener apparatus 116, in order to enable the in particular aseptic transition between the first process unit 102 and the second process unit 104.

It is understood that the transfer device 100 and in particular the further locking apparatus 330 can comprise at least one sensor unit for detecting a locked state of the second door element 112. For example, the locked state of the second door element 112 can be detectable by the at least one sensor unit by means of a position detection. For example, the locked state of the second door element 112 can be detectable by the at least one sensor unit by means of an in particular mechanical or electrical contact detection between the second door element 112 and the second process unit 104 and in particular the respective access opening 118, 124. For example, the locked state of the second door element 112 can be detectable by the at least one sensor unit inductively and/or optically and/or capacitively. It can be provided that the at least one sensor unit is connected for signaling to the control unit 146 for detecting the locked state of the second door element 112.

Referring to FIG. 15, schematically shown there is a possible further locking apparatus 330 by way of example.

The further locking apparatus 330 of FIG. 15 is designed as a bayonet apparatus.

Female bayonet elements 332 are provided in and/or on the lever element 148.

Accordingly, male bayonet counter-elements 334 are able to be disposed, or in the installed state are disposed, in and/or on the second process unit 104, in order to be able to be brought to engage with the bayonet elements 332.

The bayonet apparatus and in particular the bayonet counter-elements 334 are motorized (cf. arrow in FIG. 15), in order to be able to bring the bayonet elements and bayonet counter-elements to engage in a rotating manner for locking and/or for releasing the second door element 112.

The motorized bayonet counter-elements 334 are actuatable by means of the control unit 146 for locking and/or for releasing the second door element 112.

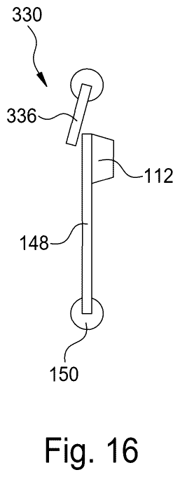

Referring to FIG. 16, schematically shown there is a possible further locking apparatus 330 by way of example.

The further locking apparatus 330 of FIG. 16 is designed as a clamping and/or tensioning apparatus.

A clamping and/or tensioning lever 336 is able to be disposed, or in the installed state is disposed, in and/or on the second process unit 104, in order to be able to be brought to engage with the second door element 112 and in particular the lever element 148, and to exert on those selectively a force directed toward the access opening 124 of the second process unit 104 for locking the second door element 112.

The clamping and/or tensioning apparatus and in particular the clamping and/or tensioning lever 336 are motorized in order to be able to bring the clamping and/or tensioning lever 336 to engage with the second door element 112 and in particular the lever element 148.

The motorized clamping and/or tensioning apparatus, in particular the motorized clamping and/or tensioning lever 336, is/are actuatable by means of the control unit 146 for locking and/or for releasing the second door element 112.

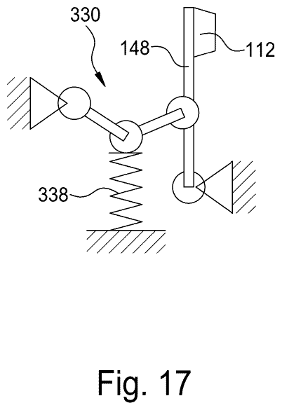

Referring to FIG. 17, schematically shown there is a possible further locking apparatus 330 by way of example.

The further locking apparatus 330 of FIG. 17 is designed as a bell-crank lever apparatus.

The bell-crank lever apparatus is able to be disposed, or in the installed state is disposed, in and/or on the second process unit 104, in order to keep the second door element 112 in a, or the, locked state.

The bell-crank lever apparatus comprises a spring element 338 by means of which the second door element 112 is able to be preloaded, or is preloaded, into the locked state.

The bell-crank lever apparatus is connected to the second door element 112, in particular to the lever element 148, and in the installed state connected to the second process unit 104.

The spring element 338 is connected to the remainder of the bell-crank lever apparatus and, in the installed state, to the second process unit 104.

By means of the bell-crank lever apparatus, a force directed toward the access opening 124 of the second process unit 104 for locking the second door element 112 is able to be exerted.

By means of the motor apparatus 208, this force is able to be overcome for opening the access opening 124 of the second process unit 104.

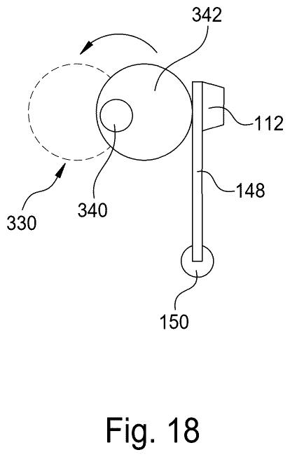

Referring to FIG. 18, schematically shown there is a possible further locking apparatus 330 by way of example.

The further locking apparatus of FIG. 18 is designed as an eccentric apparatus.

The eccentric apparatus comprises a shaft 340 on which an eccentric element 342 is formed.

By means of a rotation (cf. arrow in FIG. 18) of the shaft 340, a portion of the eccentric element 342, which is eccentric relative to the shaft 340, is able to be brought to engage with the second door element 112 and in particular the lever element 148, for locking the second door element 112, in particular in order to exert on those a force directed toward the access opening 124 of the second process unit 104 for locking the second door element 112.

The eccentric apparatus and in particular the shaft 340 are motorized in order to be able to bring the eccentric element 342 to engage with the second door element 112, in particular the lever element 148.

The motorized eccentric apparatus and in particular the motorized shaft 340 are actuatable by means of the control unit 146 for locking and/or for releasing the second door element 112.

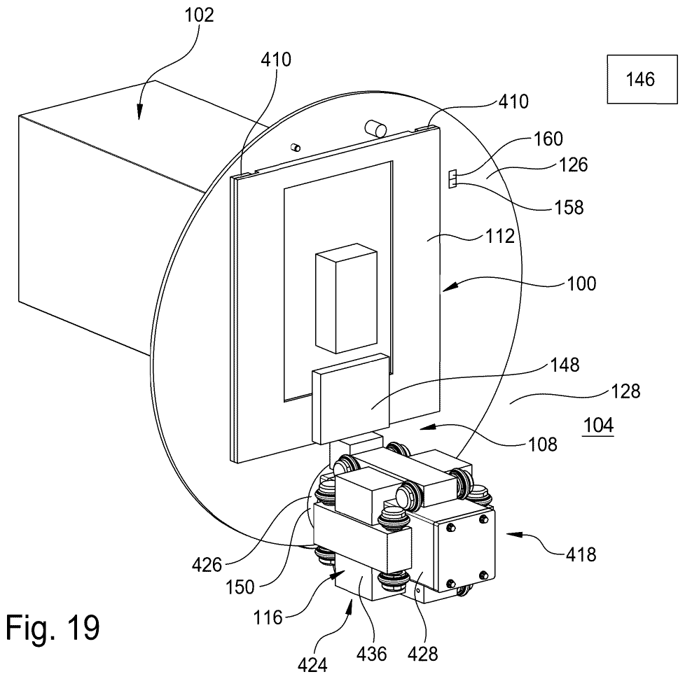

Referring to FIGS. 19 to 33, schematically illustrated there is a transfer device 100 according to a further exemplary embodiment, for enabling an in particular aseptic transition between a, e.g. first, process unit 102 and another, e.g. second, process unit 104.

The transfer device 100 of FIGS. 19 to 33 is designed substantially like the transfer device of FIGS. 1 to 7, so that only the differences are described below. In all other respects, reference is made to the above description.

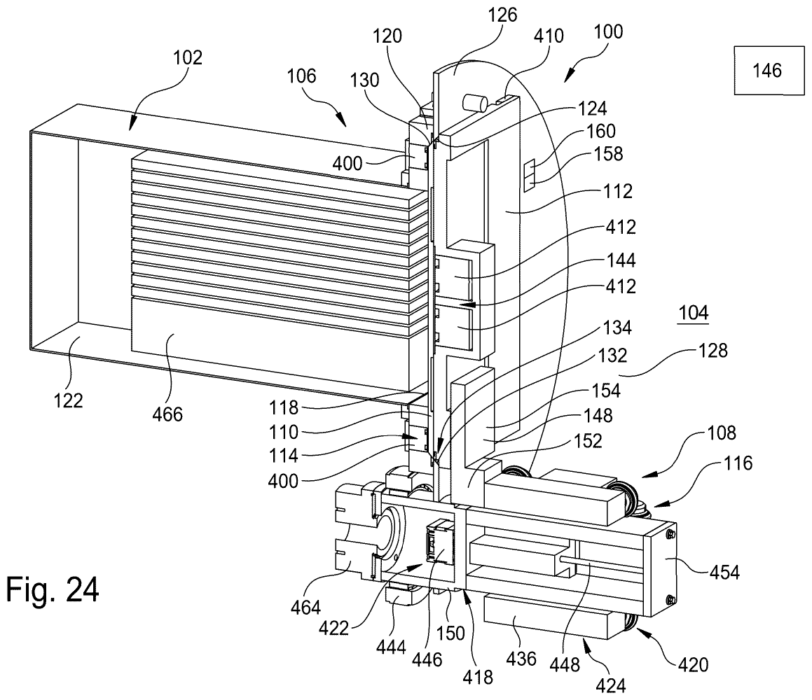

As can be seen in particular in FIG. 24, the transfer device 100 has a first door element 110, a second door element 112, a locking apparatus 114 for the first door element 110, a further locking apparatus 330 for the second door element 112 and an opener apparatus 116.

As can be seen in particular in FIG. 24 and FIGS. 28 to 30, the first door element 110 comprises a truncated pyramidal portion 130 with a rectangular base area.

The truncated pyramidal portion 130 of the first door element 110 tapers in a direction which in the closed state of the first door element 110 is directed to the interior 122 of the first process unit 102.

A circumferential surface of said portion 130 of the first door element 110 in the closed state of the first door element 110 rests on an inner circumferential surface of the access opening 118 of the first process unit 102.

Furthermore, it is particularly apparent in FIGS. 24, 28, 30 and 31 that the second door element 112 comprises a truncated pyramidal portion 132 with a rectangular base area.

The truncated pyramidal portion 132 of the second door element 112 tapers in a direction which, in the closed state of the second door element 112, is directed outward relative to the second process unit 104.

A circumferential surface of said portion 132 of the second door element 112 in the closed state of the second door element 104 rests on an inner circumferential surface of the access opening 124 of the second process unit 104.

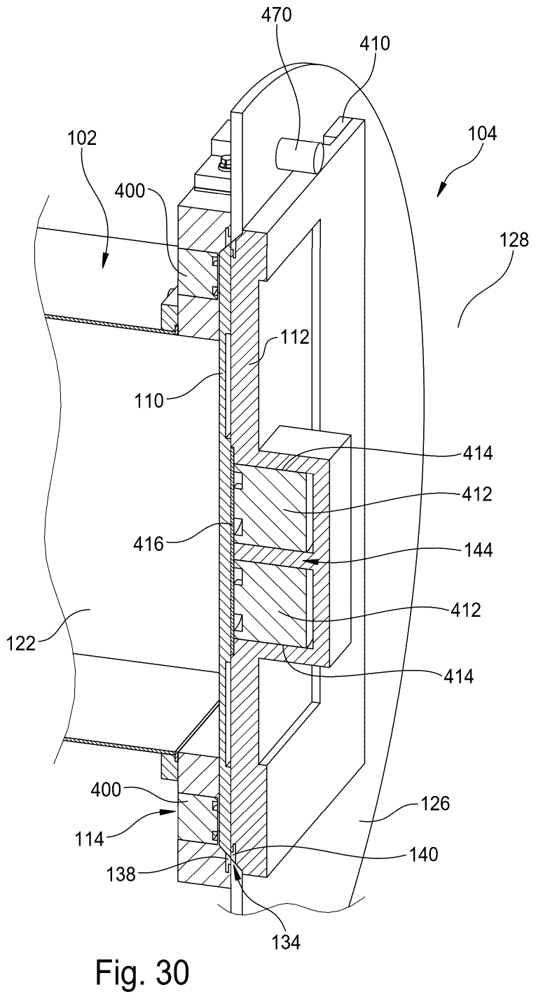

As can be readily seen in FIG. 24 in combination with FIG. 30, the sealing apparatus 134, which is comprised by the transfer device 100, presently has two sealing sub-elements 138, 140, specifically the second and the third sealing sub-element 138, 140, and/or is formed from these.

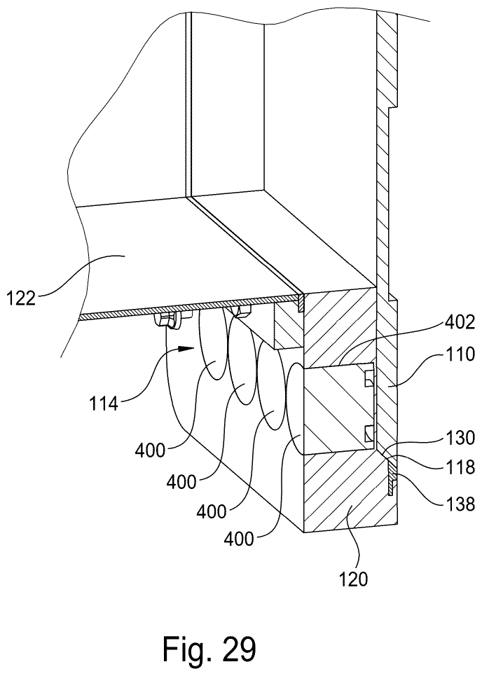

By means of the sealing apparatus 134, in the closed state of the first door element 110, the first door element 110 and an inner circumferential surface of the access opening 118 of the first process unit 110 are able to be sealed, or are sealed, in relation to the outside (cf. FIG. 29).

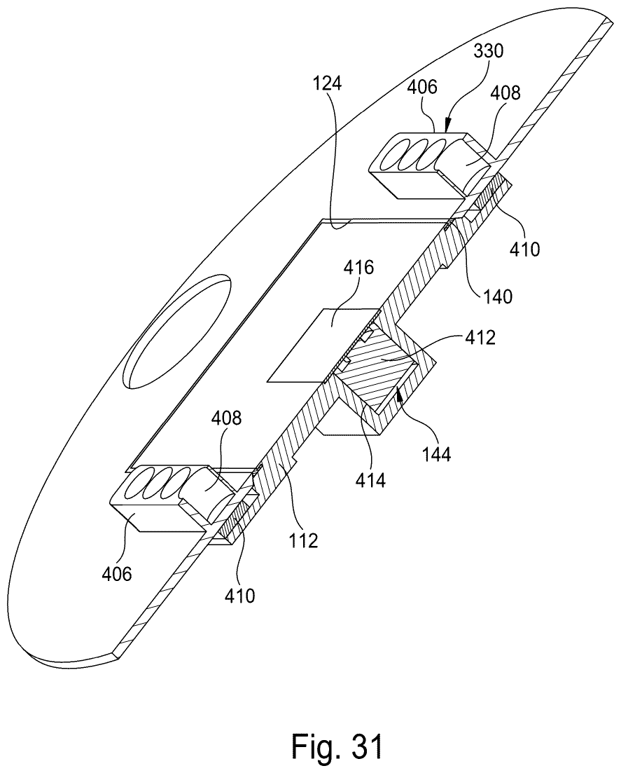

Furthermore, by means of the sealing apparatus 134, in the closed state of the second door element 112, the second door element 112 and an inner circumferential surface of the access opening 124 of the second process unit 104 are able to be sealed, or are sealed, in relation to the outside (cf. FIG. 31).

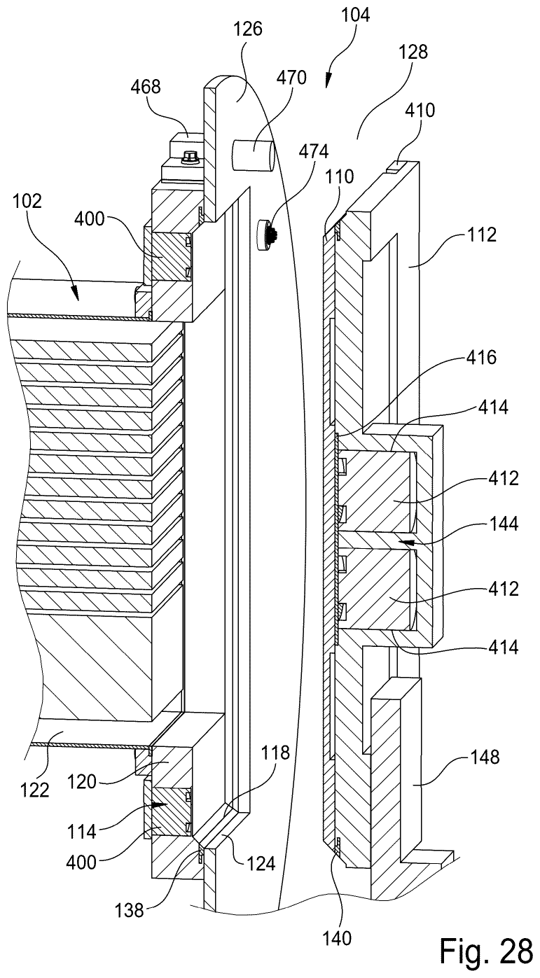

Furthermore, by means of the sealing apparatus 134, in the engaged state of the first and the second door element 110, 112, respective surfaces of both door elements 110, 112, which in the respective closed state of the corresponding door elements 110, 112 are directed outward relative to the corresponding process unit 102, 104, are able to be sealed, or are sealed, in relation to the outside (cf. FIG. 28).

Furthermore, by means of the sealing apparatus 134, in a state in which the respective wall portions 120, 126 of the process units 102, 104 rest on one another for bringing the first and the second door element 110, 112 to engage with one another (cf. FIG. 30), respective opening regions of both access openings 118, 124 of both process units 102, 104 are able to be sealed, or are sealed, in relation to the outside.

The sealing apparatus 134, in particular each sealing sub-element 138, 140, has a closed and quadrangular contour.

The second sealing sub-element 138 is able to be disposed, or is disposed, on an edge region of the access opening 118 of the first process unit 102.

This edge region of the access opening 118 of the first process unit 102 is, in the closed state of the first door element 110, adjacent to the first door element 110 and in particular to an edge region of the first door element 110, and is directed outward relative to the first process unit 102.

The contour of the second sealing sub-element 138 corresponds substantially to the edge region of the access opening 118 of the first process unit 102.

The third sealing sub-element 140 is disposed on a peripheral region of the second door element 112.

This peripheral region of the second door element 112 is, in the closed state of the second door element 112, adjacent to the inner circumferential surface of the access opening 124 of the second process unit 104 and directed outward relative to the second process unit 104.

The contour of the third sealing sub-element 140 corresponds substantially to the peripheral region of the second door element 112.

By means of the third sealing sub-element 140, the respective surfaces of both door elements 110, 112, which in the respective closed state of the corresponding door elements 110, 112 are directed outward relative to the corresponding process unit 102, 104 and are bordered by the third sealing sub-element 140 on the second door element 112, are able to be sealed, or are sealed, in relation to the outside.

This can be understood to mean in particular that a respective surface of the mutually engaged door elements 110, 112 which face one another in the engaged state of the door elements 110, 112 and/or on which the door elements 110, 112 rest on one another, is able to be sealed, or is sealed, in relation to the outside by means of the third sealing sub-element 140.

By means of the second sealing sub-element 138, respective opening regions of both access openings 118, 124 of both process units 102, 104 are able to be sealed, or are sealed, in relation to the outside when the two process units 102, 104 are coupled to one another.

In the present exemplary embodiment, a respective sealing sub-element 138, 140 is able to be manufactured, or is manufactured, from a plastics material. For example, a respective sealing sub-element 138, 140 can comprise polytetrafluoroethylene (PTFE) and/or polyvinyl chloride (PVC) and/or silicone, and/or be at least partially formed therefrom. This can advantageously enable a positive autoclaving capability.

The locking apparatus 114 serves for locking the first door element 110 in a closed state and for releasing the first door element 110 for opening the latter.

The locking apparatus 114 comprises a plurality of, presently by way of example 14, magnetic elements 400.

A magnetic element 400 is designed as a solenoid.

In the de-energized state of the magnetic elements 400, the first door element 110 is kept in the closed state. In other words, energizing can initiate a release of the first door element 110.

The magnetic elements 400 are disposed in the assigned wall portion 120 of the first process unit 102, in receptacle bores 402 provided in the latter.

The receptacle bores 402 and thus the magnetic elements 400 are configured at the assigned access opening 118.

The magnetic elements 400 face the access opening 118. When the first door element 110 is located in the access opening 118, the magnetic elements 400 face the door element 110 and are disposed directly adjacent thereto. It can be provided that the door element 110 in this state can rest on the magnetic elements 400 or can be spaced apart from the magnetic elements 400 while maintaining a magnetic operative connection, e.g. holding force, therebetween.

The receptacle bores 402 and thus the magnetic elements 400 are configured in particular on an upper and a lower region of the access opening 118.

The magnetic elements 400 are thus disposed on opposite sides relative to the access opening 118.

In the present exemplary embodiment, seven magnetic elements 400 are disposed on each side.

The magnetic elements 400 are disposed equidistantly and are presently disposed next to one another so as to be substantially directly adjacent to one another.

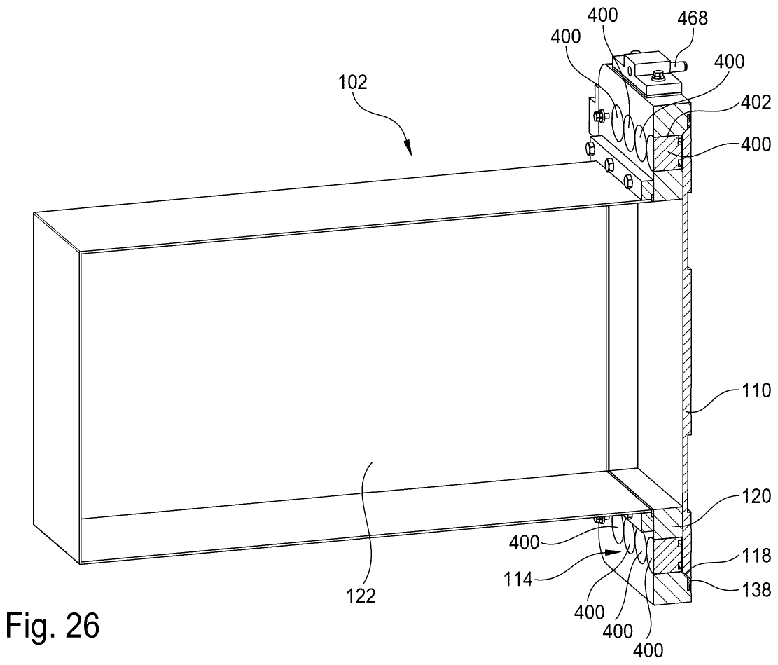

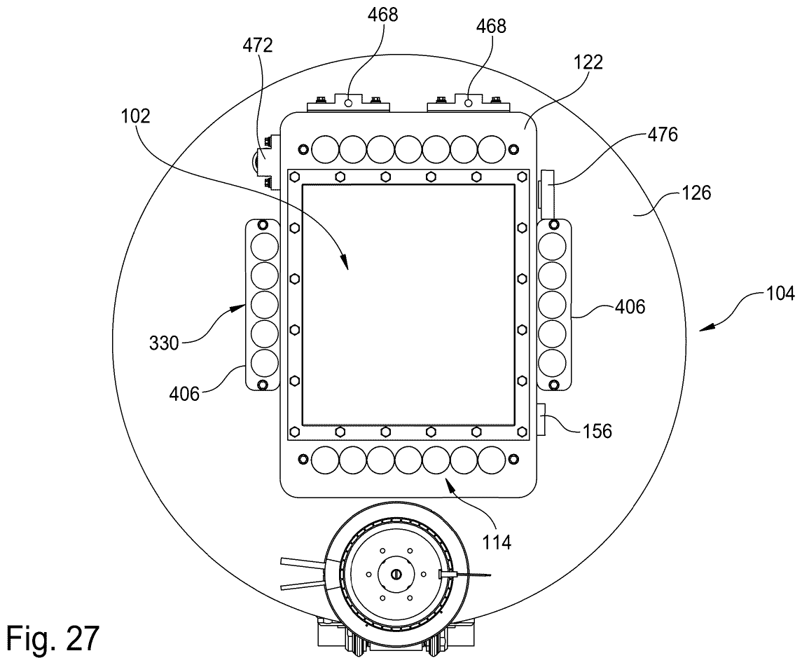

As can be seen, for example, in FIGS. 26 and 27, the magnetic elements 400 are provided in a substantially horizontal arrangement in the assigned wall portion 120.

The first door element 110 is presently designed as a magnetic counter-element, so that it can be attracted by means of the magnetic elements 400 for locking it in the closed state and can also be released again for opening it.

Advantageously, by means of the magnetic elements 400, a contact pressure and/or maintaining tightness of the first door element 110 relative to the access opening 118 can be effected based on a magnetic force. In other words, the locking apparatus 114 can function as a contact pressure apparatus for generating a contact pressure and/or for keeping the first door element 110 tight relative to the wall portion 120 and the access opening 118.

In other words, the first door element 110 is able to be pushed against the second sealing sub-element 138 based on a magnetic force by means of the locking apparatus 114, as a result of which reliable sealing therebetween can be enabled.

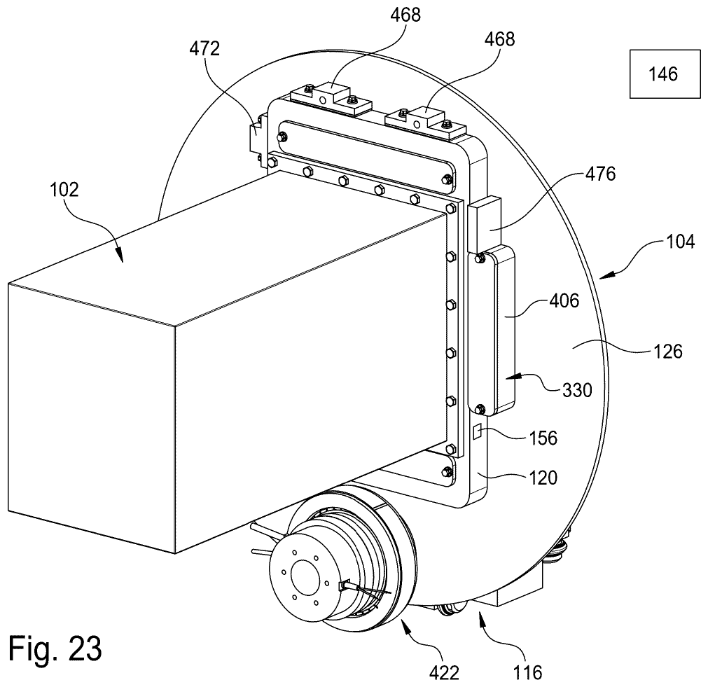

As can be seen in particular in FIG. 23, the receptacle bores 402 above the access opening 118 and below the access opening 118 are covered externally by a respective cover element fastened thereto, in order to be able to ensure a secure seating of the magnetic elements 400. Alternatively, the magnetic elements 400 can also be held in an alternative manner in the receptacle bores 402, e.g. adhesively bonded in the latter.

The further locking apparatus 330 serves for locking the second door element 112 in a closed state and for releasing the second door element 112 for opening the latter.

The further locking apparatus 330 in the installed state is disposed in and/or on the second process unit 104.

The further locking apparatus 330 is designed substantially like the further locking apparatus 330 of FIGS. 15 to 18, so that only the differences are described in the following.

The further locking apparatus 330 comprises a plurality of, presently by way of example 10, magnetic elements 404.

A magnetic element 404 is designed as a solenoid.

In the de-energized state of the magnetic elements 404, the second door element 112 is kept in the closed state. In other words, energizing can initiate a release of the second door element 112.

The magnetic elements 404 are disposed on the assigned wall portion 126 of the second process unit 104, in holding components 406 which are attached thereto and have receptacle bores 408. In other words, the magnetic elements 404 are disposed in the receptacle bores 408 which are formed in the holding components 406.

The holding components 406 with the receptacle bores 408, and thus the magnetic elements 400, are configured on the assigned access opening 124.

The holding components 406 are fastened to the outside of the wall portion 126 and laterally next to the access opening 124.

The magnetic elements 404 disposed in the receptacle bores 408 of the holding components 406 face in the direction of the access opening 124, in particular toward the interior 128 of the second process unit 104.

When the second door element 112 is located in the access opening 124, the magnetic elements 404 face the door element 112 and are disposed correspondingly to a respective counter-element 410 comprised by the second door element 112, in terms of the magnetic elements 404.

In other words, the second door element 112 has a respective counter-element 410 disposed laterally to the truncated pyramidal portion 132.

The counter-elements 410 are formed as separate components and fixed on a respective portion which is lateral relative to the truncated pyramidal portion 132.

Said counter-element 410 can be able to be manufactured, or can be manufactured, for example from a magnetizable metal or alloy, e.g. magnetizable steel. The second door element 112 can be able to be manufactured, or can be manufactured, for example from PEEK.

When the second door element 112 is disposed and/or received in the access opening 124, a respective counter-element 410 is disposed correspondingly to said magnetic elements 404, so that the magnetic elements 404 and the counter-elements 410 can be mutually adjacent in such a manner that, when the second door element 112 is closed, a contact pressure and/or maintaining tightness of the second door element 112 can be effected based on a magnetic force.

The holding components 406 and receptacle bores 408 and thus the magnetic elements 404 are configured in particular on a respective lateral region of the access opening 124.

The magnetic elements 404 are thus disposed relative to the access opening 124 on opposite sides of the access opening 124.

In the present exemplary embodiment, five magnetic elements 404 are disposed on each side.

The magnetic elements 404 are disposed equidistantly and are presently disposed one on top of the other so as to be substantially directly adjacent to one another.

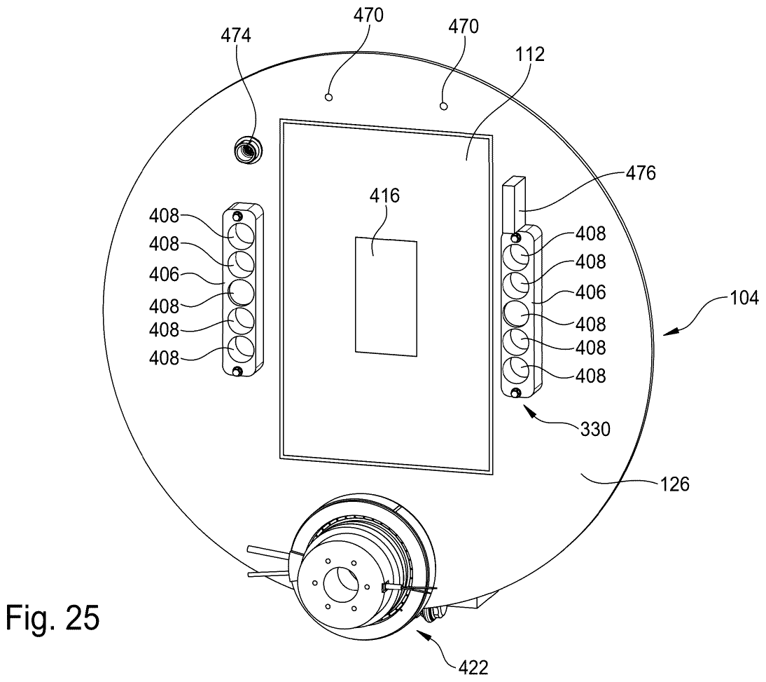

As can be seen, for example, in FIGS. 25 and 27, the magnetic elements 404 are provided in a substantially vertical arrangement on the assigned wall portion 126 in the holding components 406.

Alternatively, it can be provided that the magnetic elements 404 can be provided directly in and/or on the assigned wall portion 126.

Owing to the fact that respective counter-elements 410 are provided on the second door element 112, the second door element 112 can be attracted by means of the magnetic elements 404 for locking the latter in the closed state and can also be released again for opening the latter.

Advantageously, by means of the magnetic elements 404, said contact pressure and/or said maintaining tightness of the second door element 112 relative to the access opening 124 can be effected based on a magnetic force. In other words, the further locking apparatus 330 can function as a contact pressure apparatus for generating a contact pressure and/or for keeping the second door element 112 tight relative to the wall portion 126 and the access opening 124.

In other words, the second door element 112 with the third sealing sub-element 140 is able to be pushed against the inner circumferential surface of the access opening 124 based on a magnetic force by means of the further locking apparatus 330, as a result of which a reliable sealing therebetween can be enabled.

As can also be derived in particular from FIG. 27, the magnetic elements 400 of the locking apparatus 114 of the first door element 110 are disposed so as to be spaced apart from the magnetic elements 404 of the further locking apparatus 330 of the second door element 112. As a result, it is possible to enable a mutual spacing of their magnetic fields, so that an adverse interaction or influence between them can be avoided.

As can also be seen in FIG. 23, the receptacle bores 408 of the lateral holding components 406 are externally covered by a respective cover element attached thereto, in order to be able to ensure a secure seating of the magnetic elements 404. Alternatively, the magnetic elements 404 can also be held in an alternative manner in the receptacle bores 408, e.g. adhesively bonded in the latter.

Referring in particular to FIGS. 24, 28 and 30, the coupling element 144 of the second door element 112, by means of which the first door element 110 is able to be brought to engage with the second door element 112 and able to be held thereon, is formed from two solenoids 412.

The solenoids 412 are in each case disposed in a receptacle pocket 414 formed in the second door element 112.

The second door element 112 furthermore has a cover element 416 for covering the solenoids 412 in the receptacle pockets 414.

As can be seen in FIGS. 28 and 30, the cover element 416 forms a part of the second door element 112 directed outward relative to the second process unit 104.

This part of the second door element 112 has a flush transition to the remaining part or rest of the second door element 112, the latter being directed outward relative to the second process unit 104.

The coupling element 144 is presently disposed so as to be substantially centric in the truncated pyramidal portion 132 of the second door element 112.

Owing to the fact that the first door element 110, as already described, is designed as a magnetic counter-element—for example, can be able to be manufactured, or can be manufactured, from a magnetic or magnetizable material—the first door element 110 is able to be brought to engage with the second door element 112 and able to be held thereon by means of the coupling element 144.

The coupling element 144 formed as a solenoid 412 is actuatable by means of the control unit 146 described herein, in order to bring the first door element 110 to engage with the second door element 112 and to hold it thereon.

As already described, the control unit 146 can be comprised by the transfer device 100, and/or can be assignable, or assigned, to the transfer device 100. For example, a control unit 146 which is assignable, or assigned, to the transfer device 100 can be comprised by the first and/or the second process unit 102, 104.

It is understood that all, preferably electrically activatable, devices, apparatuses and/or components of the transfer device 100 can be connected for signaling to the control unit 146, and can be controlled by the latter for an orderly operation of the transfer device 100 and/or of the process units 102, 104.

Now referring in particular to FIGS. 19 to 24 and FIGS. 32 and 33, the opener apparatus 116 for opening and closing at least one of the door elements 110, 112 is designed differently in comparison to FIGS. 1 to 5.

As already described, the opener apparatus 116 serves for opening and closing at least one of the door elements 110, 112 and is able to be disposed, or in the installed state is disposed, in and/or on the second process unit 104.

The first door element 110 and the second door element 112 can be brought to engage with one another in order to be able to be removed conjointly from the access openings 118, 124 by means of the opener apparatus 116.

In the engaged state of the door elements 110, 112 and, when the first and the second door element 110, 112 are released by means of the locking apparatuses 114, 330, the first door element 110 and the second door element 112 are able to be removed conjointly from the access openings 118, 124 by means of the opener apparatus 116. As a result, said in particular aseptic transition between the first process unit 102 and the second process unit 104 can be enabled and/or is implemented.

The opener apparatus 116 has a drive unit 418.

The drive unit 418 is disposed and fastened in and on the second process unit 104.

In particular, the drive unit 418 is fixed in and on the wall portion 126 of the second process unit 104.

The drive unit 418 has a motion apparatus 420 and a motor apparatus 422.

The lever element 148, and thus also the second door element 112 (by way of the lever element 148), is fastened to the drive unit 418.

Specifically, the lever element 148 is fastened to the motion apparatus 420.

The motion apparatus 420 is movable relative to the wall portion 126 and to the access opening 124 by means of the motor apparatus 422.

In particular, the motion apparatus 420 is both rotatable as well as at least partially linearly movable by means of the motor apparatus 422.

In other words, the lever element 148, and thus also the second door element 112 and/or the mutually engaged door elements 110, 112, are both rotatable and linearly movable by means of the motor apparatus 422.

The motion apparatus 422 has a center of rotation 150, which is formed as a hollow shaft, and a displacement device 424.

The center of rotation 150 comprises a cylindrical portion 426 and an adjoining cuboid portion 428.

The center of rotation 150 is disposed perpendicularly to the wall portion 126 of the second process unit 104.

The center of rotation 150 extends through the wall portion 126, wherein the cuboid portion 428 is disposed completely in the interior 128 of the second process unit 104 and the cylindrical portion 426 is disposed predominantly outside the interior 128 of the second process unit 104.

The center of rotation 150 is in each case rotatably mounted in the wall portion 126 and a fastening portion 430 of the wall portion 126 by a respective bearing device 432.

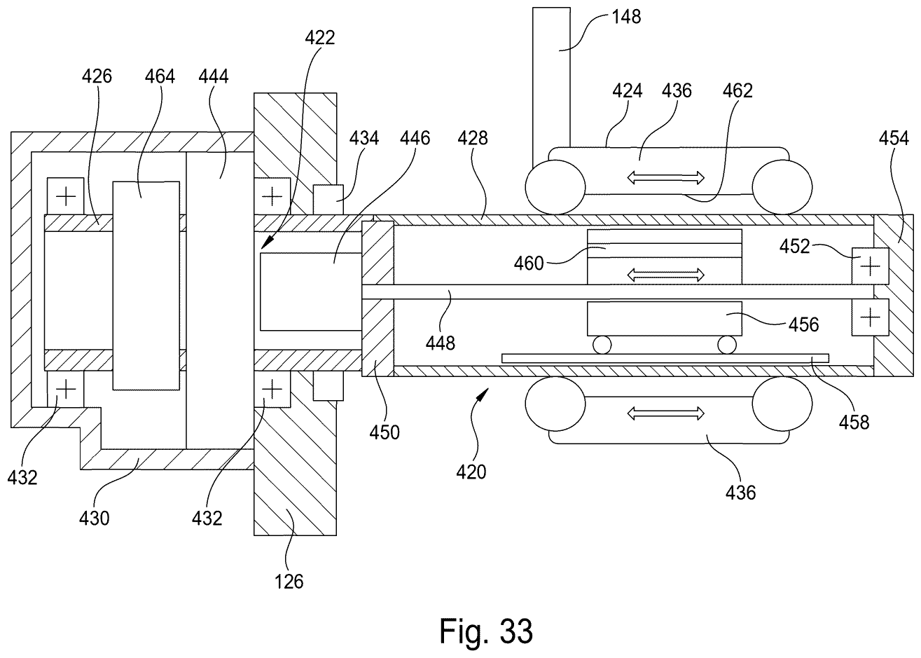

The fastening portion 430 is only shown in FIG. 33 and is omitted in the remaining figures for reasons of clarity.

The fastening portion 430 is provided externally relative to the interior 128 of the second process unit 104. The fastening portion 430 is preferably releasably fastened to the wall portion 126 and can, conjointly with the wall portion 126, form a housing, in which the motor apparatus 422 is at least partially disposed.

Specifically, a bearing device 432 is disposed between the wall portion 126 and the cylindrical portion 426, and a further bearing device 432 is disposed between the fastening portion 430 and the cylindrical portion 426.

A transition between the interior 128 and the exterior of the second process unit 104, which is obtained by extending the center of rotation 150 through the wall portion 126, is preferably aseptically sealed by means of a further sealing apparatus 434.

In other words, the drive unit 418 can have two bearing devices 432, e.g. ball bearing devices, and the further sealing apparatus 434.

The displacement device 424 is disposed externally on the center of rotation 150, in particular on the cuboid portion 428 and thus within the second process unit 104.

The displacement device 424 is linearly movable and/or displaceable along the center of rotation 150 and in particular along the cuboid portion 428, this being highlighted by the arrows illustrated in FIG. 33, for example.

The displacement device 424 is designed in the manner of a carriage, and presently has a carriage unit 436.

The carriage unit 436 is disposed externally on the cuboid portion 428 and is linearly movable and/or displaceable along the cuboid portion 428.

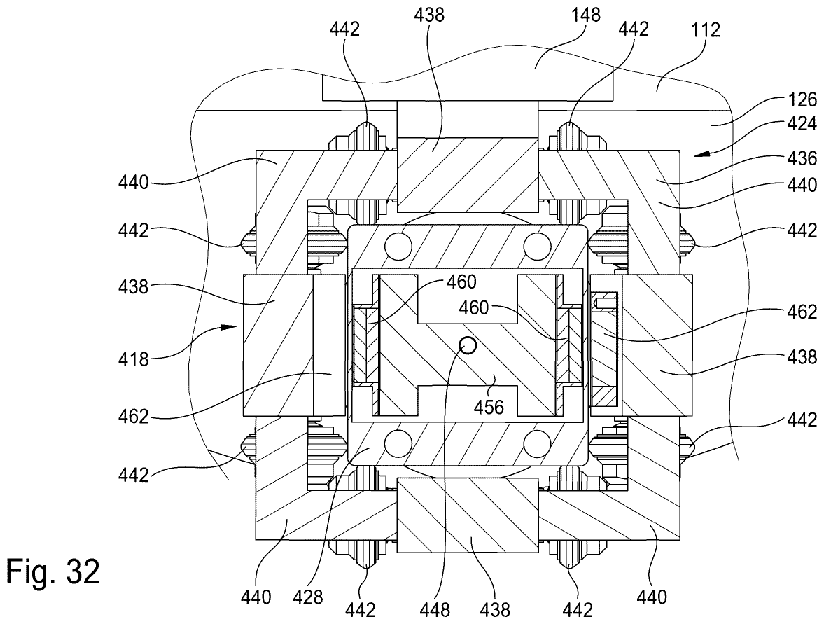

As can be seen in particular in FIG. 32, the carriage unit 436 encompasses the cuboid portion 428, or the four lateral shell surfaces of the cuboid portion 428, substantially completely.

The carriage unit 436 has four carriage portions 438 and four connecting portions 440.

A carriage portion 438 has a plurality of, presently four, roller elements 442 for rolling on the center of rotation 150, in particular on the cuboid portion 428.

One carriage portion 438 is in each case disposed on a lateral shell surface of the cuboid portion 428.

A connecting portion 440, by means of which the respective carriage portions 438 are interconnected, is disposed between respective carriage portions 438.

In other words, the connecting portions 440 bridge the edge region of the cuboid portion 428 between the individual lateral shell surfaces.

For this purpose, the connecting portions 440 have an L-shaped cross section.

As can be seen from the figures, the lever element 148 is fastened to a, or the, carriage portion 438 which in the non-activated state of the motor apparatus 422 faces the access opening 124. In the figures, for example, this is the upper carriage portion 438.

By means of the carriage unit 436, the linear mobility of the displacement device 424 along the center of rotation 150 can be enabled.

As already described, the center of rotation 150, in particular the cylindrical portion 426 and the cuboid portion 428 conjointly, and the displacement device 424 are rotatable conjointly by means of the motor apparatus 422.

Furthermore, the displacement device 424, in particular the carriage unit 436, is linearly movable and/or displaceable along the center of rotation 150, in particular along the cuboid portion 428, by means of the motor apparatus 422.

In order to be able to initiate said rotating movement, the motor apparatus 422 has a rotational motor 444. In other words, the rotational motor 444 is a rotational motor for initiating a rotating movement of the motion apparatus 420.

Furthermore, in order to be able to initiate said linear movement, the motor apparatus 422 has a linear motor, here spindle drive motor 446. In other words, the spindle drive motor 446 is a motor for initiating a linear movement of at least a part, in particular of the displacement device 424, of the motion apparatus 420. The spindle drive motor 446 has a drivable spindle 448.

In other words, the center of rotation 150 and the displacement device 424 are rotatable by means of the rotational motor 444, and the displacement device 424 is able to be initiated for linear movement by means of the spindle drive motor 446.

The motor apparatus 422, in particular the rotational motor 444 and the spindle drive motor 446, is/are controllable by means of the control unit 146.

The rotational motor 444 is fixed between the center of rotation 150, in particular the cylindrical portion 426, and the fastening portion 430 of the wall portion 126, and is preferably disposed adjacent to the wall portion 126.

In other words, the rotational motor 444 is disposed externally on the center of rotation 150, in particular the cylindrical portion 426, and fastened to the wall or the wall portion 126 of the second process unit 104.

The spindle drive motor 446 is disposed and fastened within the center of rotation 150. This can be understood to mean in particular that the spindle drive motor 446 is rotatable conjointly with the center of rotation 150.

In particular, the spindle drive motor 446 is disposed within the cylindrical portion 426 and fastened to a partition wall 450 which mutually separates the respective interior of the cylindrical portion 426 and the cuboid portion 428.

The spindle 448 extends through this partition wall 450 and is mounted by means of a spindle bearing device 452 on a cover element 454 of the cuboid portion 428 that closes the inside of the cuboid portion 428 in an end region opposite the partition wall 450 relative to the inside 128 of the second process unit 104.

A slide-type and/or carriage-type displacement element 456, which is comprised by the motion apparatus 420, is disposed in the interior of the cuboid portion 428 on a linear guide 458 disposed in the interior of the cuboid portion 428 of the motion apparatus 420, and engages with the spindle 448. It can be provided that the linear guide 458 is optional. In this case, the displacement element 456 can be linearly guided solely by the spindle 448, for example.

By a rotation of the spindle drive motor 446 and in particular of the spindle 448, the displacement element 456 is linearly movable and/or displaceable in a reciprocating manner along the spindle 448 within the cuboid portion 428.

In this case, the displacement element 456 and the displacement device 434, and in particular the carriage unit 436, are operatively connected to one another in a non-contacting and presently magnetic manner, in order to enable conjoint mobility and in particular to make it possible for a movement of the displacement element 456 to initiate a movement of the carriage unit 436.

For this purpose, the displacement element 456 has a plurality of entrainment magnets 460, and the displacement device 434 and in particular the carriage unit 436 has a plurality of corresponding magnetic and/or magnetizable counter-elements, in other words entrainment counter-elements 462, as a result of which the contactless operative connection between the displacement element 456 and the displacement device 424 is enabled.

As can be seen schematically in general in FIG. 33 and by way of example specifically in FIG. 32, entrainment magnets 460 are fixed externally on the displacement element 456 and correspondingly, entrainment counter-elements 462 are fixed internally on the carriage unit 436 and in particular on carriage portions 438, so as to be disposed opposite one another relative to a wall of the cuboid portion 428, and thus to be in mutual contactless operative connection.

As illustrated by way of example in FIG. 32, one entrainment magnet 460 is disposed on a respective side of the displacement element 456 and one entrainment counter-element 462 is disposed on a respective inward-facing side of a laterally disposed carriage portion 438.

When the displacement element 456 moves linearly (initiated by a rotation of the spindle 448), the displacement device 424, and in particular the carriage unit 436, moves correspondingly, preferably linearly, along the center of rotation 150 and in particular of the cuboid portion 428.

In particular with reference to FIG. 33 and, for example, FIG. 24, the motor apparatus 422 has a braking apparatus 464 for braking a rotation of the center of rotation 150 and/or of the motion apparatus 420.

The braking apparatus 464 is disposed on and fastened to an end region of the center of rotation 150 and, in particular, the cylindrical portion 426, which faces away from the wall portion 126.

In other words, the rotational motor 444 is disposed between the braking apparatus 464 and the wall portion 126.

By means of the braking apparatus 464, a, or the, rotating movement of the center of rotation 150, and thus also a pivoting movement of the second door element 112 or the mutually engaged door elements 110, 112, is able to be braked and preferably decelerated and/or stopped. It is understood that this can apply, or does apply, also to the other components fastened indirectly or directly to the center of rotation 150, such as the displacement device 424, for example.

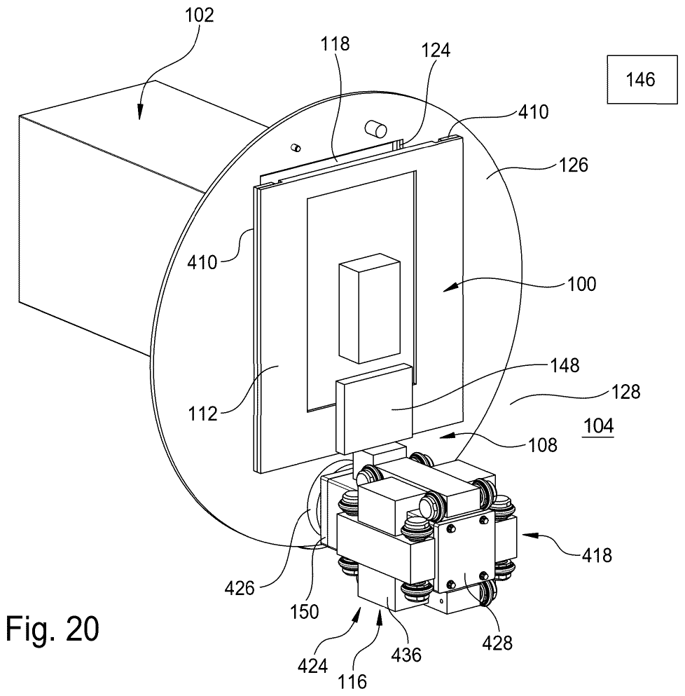

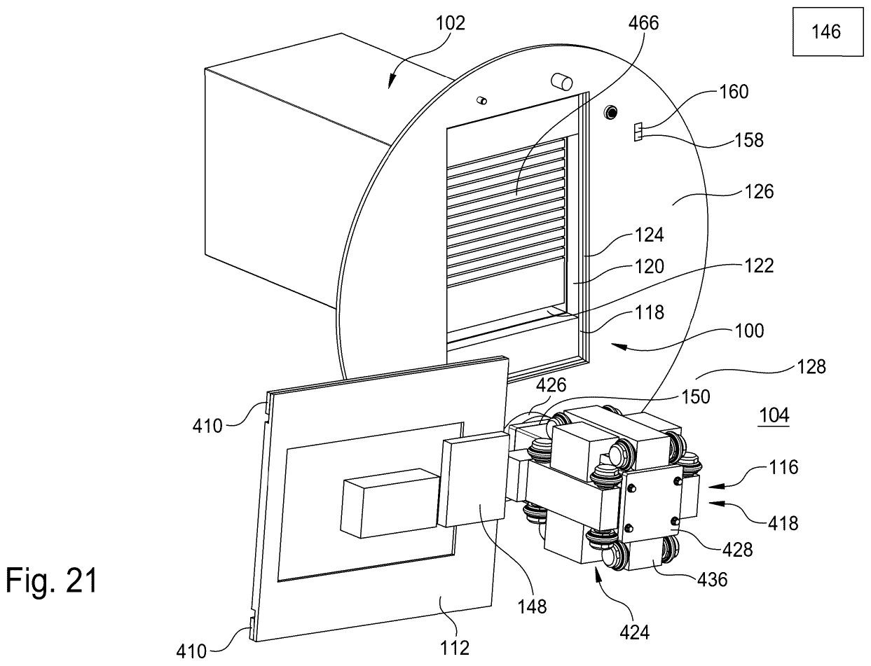

By means of the opener apparatus 116, and in particular initiated by the drive unit 418, the second door element 112 or the mutually engaged door elements 110, 112 conjointly, are movable first linearly (cf. FIGS. 19 and 20) and then in a pivoting manner (cf. FIG. 21) into the interior 128 of the second process unit 104, and thus are able to be removed from the access openings 118, 124.

In other words, the drive unit 418 can move the second door element 112, or the mutually engaged door elements 110, 112 conjointly, linearly in the direction of the interior 128 of the second process unit 104 (cf. FIGS. 19 and 20) and subsequently pivot it/them in the interior 128 (cf. FIG. 21), preferably for a, or the, opening procedure.

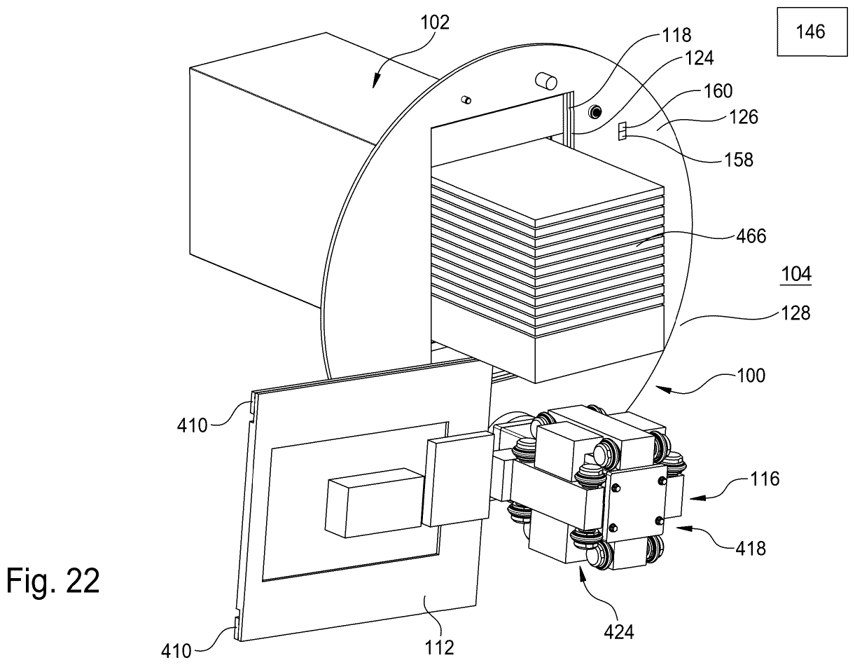

If said opening procedure is now completed, and a preferably aseptic transition between the process units 102, 104 is enabled, an exchange of treatment objects can take place between the process units 102, 104. Illustrated in FIGS. 21 and 22, for example, is a magazine device 466, for example for storing cells, which is moved in FIG. 22 from the first process unit 102 by way of a pull-out device disposed in the first process unit 102 into the second process unit 104.

The drive unit 418 can pivot the second door element 112, or the mutually engaged door elements 110, 112 conjointly, in the interior 128 of the second process unit 104, and subsequently move it/them linearly in the direction of the access opening 124 and preferably into the latter. This is preferably a, or the, closing procedure.

As already described, a process unit 102, 104 can have one or a plurality of coupling devices, by means of which the process unit 102, 104 is able to be connected, for example mechanically coupled, to one or a plurality of further process units 102, 104.

In the present exemplary embodiment, a coupling device of positioning elements 468 for positioning the first process unit 102 with the second process unit 103 is formed, which are disposed and/or fastened and/or formed on the first process unit 102 and in particular on the wall portion 120.

The second process unit 104 has positioning elements 470 which are complementary to the positioning elements 468 and are disposed and/or fastened and/or formed on the wall portion 126 of the second process unit 104 so as to correspond to the positioning elements 468.

Such a positioning element 468 is formed as a positioning protrusion for insertion into a positioning receptacle.

Such a complementary positioning element 470 is designed as a positioning receptacle for receiving a positioning protrusion.

Furthermore, a further coupling device in the present exemplary embodiment is formed by a plug element 472 and a socket element 474.

The plug element 472 and the socket element 474 serve to transfer energy and/or fluids, in particular gases, gas mixtures and/or liquids, between coupled process units 102, 104.

The plug element 472 is disposed and/or fastened and/or formed on the first process unit 102 and in particular on the wall portion 120.

The socket element 474 is disposed and/or fastened and/or formed on the second process unit 102 and in particular on the wall portion 126, so as to correspond to the plug element 472.

It can be provided that the coupling devices, in particular said positioning elements 468, 470 and the plug element 472 and the socket element 474 are able to be assigned, or are assigned, to the transfer device 100, and/or can be comprised by the latter.

The transfer device 100 furthermore has a further, e.g. fifth, sensor unit 476 for presence detection and/or identification of a first process unit 102.

In the present exemplary embodiment, this fifth sensor unit 476 is fixed on the wall portion 126 of the second process unit 104 and adjacent to a holding component 406 of the further locking apparatus 330.

The fifth sensor unit 476 is connected to the control unit 146 for signaling.

When the fifth sensor unit 476 detects the first process unit 102 in a state ready for coupling of the door elements 110, 112, the control unit 146 can actuate the coupling element 144 accordingly and subsequently initiate an opening procedure of the door elements 110, 112.

For example, the fifth sensor unit 476 can be designed as and/or comprise an RFID sensor to detect additional information about the first process unit 102, for example about its content. It is understood that the first process unit 102 can be or is provided with a corresponding RFID tag.

An operation, in particular an opening procedure, of the transfer devices 100 is now briefly described below:

In an initial state, the first and second process units 102, 104 are coupled to one another (cf. FIGS. 19, 23, 24 and 30) in order to enable a preferably aseptic transition.

The control unit 146 actuates the coupling element 144 so as to connect the first and second door elements 110, 112 to one another and/or to bring them to engage with one another.

Subsequently, the first door element 110 is released by means of the locking apparatus 114, preferably in response to a corresponding actuation by the control unit 146.

Simultaneously or subsequently, the second door element 112 is released by means of the further locking apparatus 330, preferably in response to a corresponding actuation by the control unit 146.

The first and second door elements 110, 112 are now engaged with one another and are no longer locked.

By means of the spindle drive motor 446, the spindle 448 is rotated, whereupon the displacement element 456 is moved linearly and away from the wall portion 126.

By way of the contactless operative connection between the displacement element 456 and the carriage unit 436, the carriage unit 436 is correspondingly moved linearly, and thus also the door elements 110, 112 which are connected to the carriage unit 436 by way of the lever element 148 and are mutually engaged. As a result, the mutually engaged door elements 110, 112 are moved out of the corresponding access openings 118, 124 (cf. FIGS. 20, 28 and 33).

Subsequently, by means of the rotational motor 444, the center of rotation 150 is rotated and the carriage unit 436 disposed thereon, and thus the door elements 110, 112, are pivoted. As a result, the mutually engaged door elements 110, 112 are pivoted away from the corresponding access openings 118, 124 (cf. FIG. 21).

An obstacle-free and preferably aseptic transition between the process units 102, 104 is now provided, so that, for example, a magazine device 466 from the first process unit 102 can be transferable, or can be transferred, to the second process unit 104 (cf. FIG. 22).

Conversely, the process units 102, 104 can be closed again.

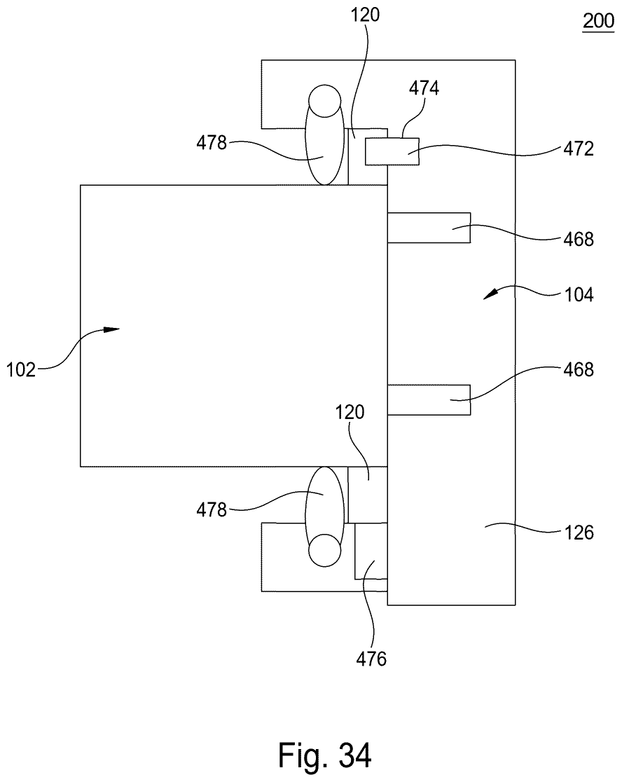

Referring to FIG. 34, shown there is a system 200 consisting of two process units 102, 104 which are substantially configured and formed as described in the context of FIGS. 19 to 33, so that only the differences are explained in the following.

As can be seen in FIG. 34, the second process unit 104 has the coupling devices already described, which are formed by the complementary positioning elements 470 and the socket element 474 respectively. Accordingly, the first process unit 102 comprises the positioning elements 468 and the plug element 472.

The second process unit 104 furthermore presently comprises a further coupling device which is formed from a plurality of, presently two, locking elements 478 for locking and/or interlocking the first process unit 102 to the second process unit 104.

The locking elements 478 are disposed externally on the wall portion 126 of the second process unit 104 and opposite one another relative to the access opening 124 of the second process unit 104.

The locking elements 478 are each formed as pivotable holding elements by means of which lateral peripheral regions of the wall portion 120 of the first process unit 102 are able to be engaged in from the rear. Such a state, in which the wall portion 120 of the first process unit 102 is engaged in from the rear by means of the locking elements 478, is illustrated by way of example in FIG. 34.

In other words, the lateral peripheral regions of the wall portion 120 of the first process unit 102 serve as holding contours for receiving the locking elements 478.

In other words, such a locking element 478 of the second process unit 104 is formed as a holding element for engaging in a holding contour, and a locking element of the first process unit 102 which is complementary to the locking element 478 is formed as a holding contour for receiving the holding element.

Presently, the locking elements 478 are motorized so that preferably a locking and/or interlocking can be carried out automatically and in particular fully automatically.

Advantageously, the first process unit 102 is able to be attracted to the second process unit 104 by means of the locking elements 478 and able to be held thereon, as a result of which a compressive force is able to be exerted, thus making it possible to enable a reliable and/or sealed coupling of the process units 102, 104.

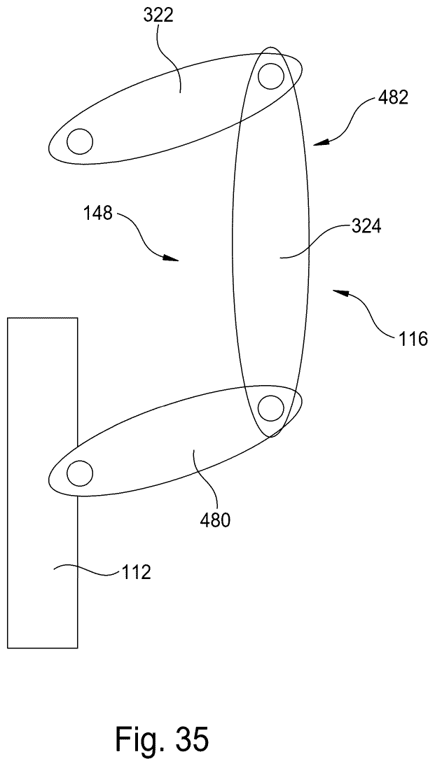

Referring to FIG. 35, shown there is a part of a transfer device 100 according to a further exemplary embodiment, in which the opener apparatus 116, and in particular the lever element 148, are formed differently in comparison to the preceding figures.

The opener apparatus 116 illustrated in FIG. 35 is formed substantially like the opener apparatus 116 illustrated in FIG. 12, so that only the differences are described in the following.

The pivotable lever element 148 is formed from a plurality of, here three, lever links 322, 324, 480.

The lever links 322, 324, 480 are movably connected to one another.

Presently, the pivotable lever element 148 is thus formed from a first, a second and a third lever link 322, 324, 480.

In the installed state in the second process unit 104, the first lever link 322 is pivotably fixed on a first end region of the first lever link 322, and a first end region of the second lever link 324 is pivotably fixed on a second end region of the first lever link 322.

A first end region of the third lever link 480 is pivotably fixed on a second end region of the second lever link 324, and a second end region of the third lever link 480 is pivotably fixed on the second door element 112.

By means of the lever links 322, 324, 480, the second door element 112 or the mutually engaged door elements 110, 112 conjointly, is/are able to be moved into the interior 128 of the second process unit 104 and thus removed from the access openings 118, 124.

A first motor (not shown in the figures) is disposed between the second process unit 104 and the first lever link 322, and optionally a second motor (not shown in the figures) is disposed between the first lever link 322 and the second lever link 324.

The first, and optionally the second, motor can form said motor apparatus 208 and/or be comprised by the latter.

Optionally, a third motor, which for example can likewise be assignable, or assigned, to the motor apparatus 208, can be provided between the second and the third lever link 324, 480.

Preferably, the first, the second and the third lever link 322, 324, 480 are disposed in a parallelogram arrangement and form a parallelogram guide 482.

The parallelogram arrangement can advantageously be space-saving in the interior 128 of the second process unit 104.

By means of the parallelogram arrangement or parallelogram guide 482, the second door element 112 or the mutually engaged door elements 110, 112 conjointly, is/are able to be moved into the interior 128 of the second process unit 104 and thus able to be removed from the access openings 118, 124.

The movement guided by means of the parallelogram arrangement or parallelogram guide 482 is advantageously a two-dimensional linear movement in a plane.

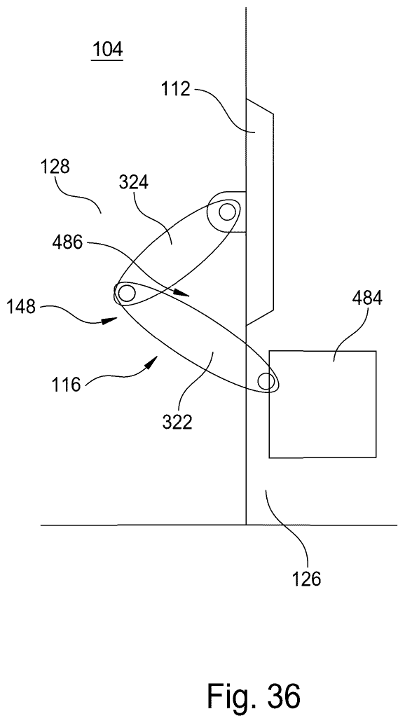

Referring to FIG. 36, shown there is a part of a transfer device 100 according to a further exemplary embodiment, in which the opener apparatus 116, and in particular the lever element 148, are formed differently in comparison to the preceding figures.

The opener apparatus 116 illustrated in FIG. 36 is formed substantially like the opener apparatus 116 illustrated in FIG. 12, so that only the differences are described in the following.

A motor 484 is disposed between the second process unit 104, in particular the wall portion 126, and the first lever link 322.

The first lever link 322 is connected by way of the motor 484 to the second process unit 104, in particular the wall portion 126.

The first lever link 322 and the second lever link 324 form a lever kinematics 486.

By means of the lever kinematics 486, the second door element 112 disposed on the second lever link 324, or the mutually engaged door elements 110, 112 conjointly, is/are able to be moved linearly into the interior 128 of the second process unit 104 and subsequently able to be pivotably or linearly moved in the interior 128, as a result of which an unobstructed access of the assigned access opening 118, 124 can be enabled.

In other words, the second door element 112 disposed on the second lever link 324, or the mutually engaged door elements 110, 112 conjointly, is/are able to be first lifted out of the access openings 118, 124 and subsequently moved laterally, as a result of which the unobstructed access to the access openings 118, 124 can be enabled.

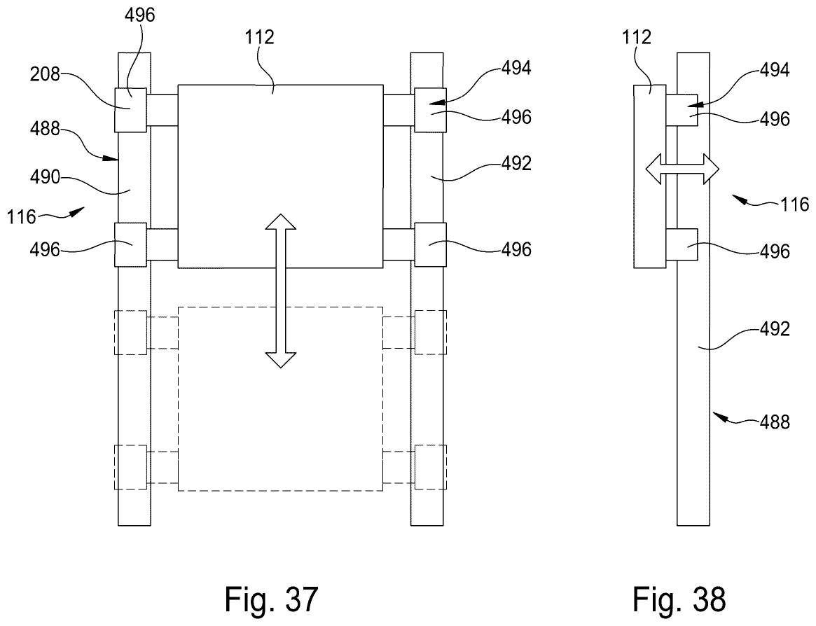

Referring to FIGS. 37 and 38, shown there is a part of a transfer device 100 according to a further exemplary embodiment, in which the opener apparatus 116 is designed differently in comparison to the preceding figures.

The opener apparatus 116 has a guide apparatus 488 and a motor apparatus 208.

The second door element 112 is fixed on the guide apparatus 488.

The guide apparatus 488 is disposed adjacent to the access opening 124.

By means of the motor apparatus 208, the second door element 112 is able to be moved linearly along a part of the guide apparatus 488 and out of the associated access opening 124 (cf. arrow in FIG. 38), and subsequently able to be moved along a further part of the guide apparatus 488 linearly away from the associated access opening 124 (cf. arrow in FIG. 37).

The guide apparatus 488 has a first guide element 490 and a second guide element 492, which are in each case disposed on mutually opposite sides of the access opening 124 in the interior 128 of the second process unit 104.

The first and the second guide element 490, 492 are disposed substantially parallel to one another and to the access opening 124.

The first and the second guide element 490, 492 are presently in each case designed as a guide railed designed as a magnetic rail.

The first and the second guide element 490, 492 are substantially the same length.

The second door element 112 is held so as to be moveable on the first and the second guide element 490, 492.

In particular, the second door element 112 is held on the first and the second guide element 490, 492 based on a magnetic force.

The guide apparatus 488 furthermore comprises a holding kinematics 494 by way of which the second door element 112 is movably held on the guide elements 490, 492.

As can be seen from the respective figure, the second door element 112 is movably held on the guide elements 490, 492 by way of a plurality of, presently four, holding links 496 of the holding kinematics 494.

The holding links 496 of the holding kinematics 494 are in each case presently designed as and/or function as and/or comprise counter-elements to the magnetic rail.

The holding links 496 of the holding kinematics 494 are preferably designed in the manner of slides.

Presently, the number of holding links 496 per guide element 490, 492 by way of which the second door element 112 is movably held on the guide elements 490, 492 is the same, presently two.

By means of the motor apparatus 208, the second door element 112 is able to be moved linearly out of the access opening 124 and/or back into the latter by way of the holding kinematics 494 (cf. arrow in FIG. 38).

In other words, the holding kinematics 494 is able to be activated by means of the motor apparatus 208 in such a manner that the second door element 112 can be initiated to move linearly out of the access opening 124 and/or back into the latter.

The second door element 112, in a state in which it has been moved out of the access opening 124, can be moved away from the access opening 124 and/or returned to the latter by means of the motor apparatus 208 by way of the guide elements 490, 492 (cf. arrow in FIG. 37).

In other words, the holding kinematics 494, in a state in which the second door element 112 has been moved out of the access opening 124, is able to be activated by means of the motor apparatus 208 in such a manner that the second door element 112 can be initiated to move linearly along the guide elements 490, 492.

In particular, the second door element 112 is able to be moved away from the associated access opening 124 by a movement which lies in a plane that is oriented transversely, in particular perpendicularly, to the previous movement.

For example, vectors of the respective movements can be mutually perpendicular.

As can be seen in the respective figures, the movement from the access opening 124 is oriented essentially horizontally (cf. arrow in FIG. 38), and the subsequent movement away from the access opening 124 is oriented substantially perpendicularly (cf. arrow in FIG. 37). This is preferably an opening procedure. The open state is illustrated by dashed lines in FIG. 37.

The second door element 112 is able to be moved in an analogous manner back into the access opening 124; in other words, the second door element 112 is able to move back along the previously described movement path. This is preferably a closing procedure.

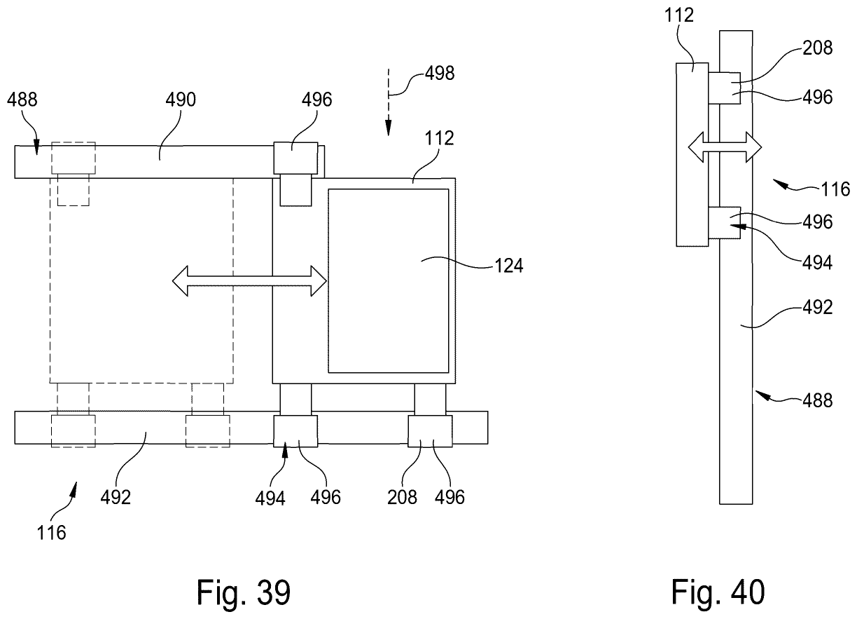

Referring to FIGS. 39 and 40, shown there is a part of a transfer device 100 according to a further exemplary embodiment, in which the opener apparatus 116 is designed differently in comparison to the preceding figures.

The opener apparatus 116 illustrated in FIGS. 39 and 40 is formed substantially like the opener apparatus 116 illustrated in FIGS. 37 and 38, so that only the differences are described below.

In comparison to the opener apparatus 116 illustrated in FIGS. 37 and 38, for example, the opener apparatus 116 can be disposed so as to be rotated by 90°.

It is understood that the respective movements which initiate the second door element 112 to open and/or close the access opening 124 are correspondingly oriented differently.

Furthermore, the first guide element 490, which is in terms of a height direction is disposed above the second guide element 492, is shorter, preferably at least by a length of the access opening 124 in a direction transverse and in particular perpendicular to the height direction.

Presently, the second door element 112 is moveably held on the first guide element 490 by way of one holding link 496, and is movably held on the second guide element 492 by way of two holding links 496. In other words, the number of holding links 496 per guide element 490, 492 differs.

Owing to the fact that the first guide element 490 is of a shorter design, and the second door element 112 thus is not guided above the access opening 124, a laminar flow 498 which can be enabled in the second process unit 104 can advantageously flow unobstructed by way of the second door element 112 and/or the access opening 124, and/or a particle contamination from above the access opening 124, e.g. due to trickling, can preferably be avoidable.

In all other respects, reference is made to the description of FIGS. 37 and 38.

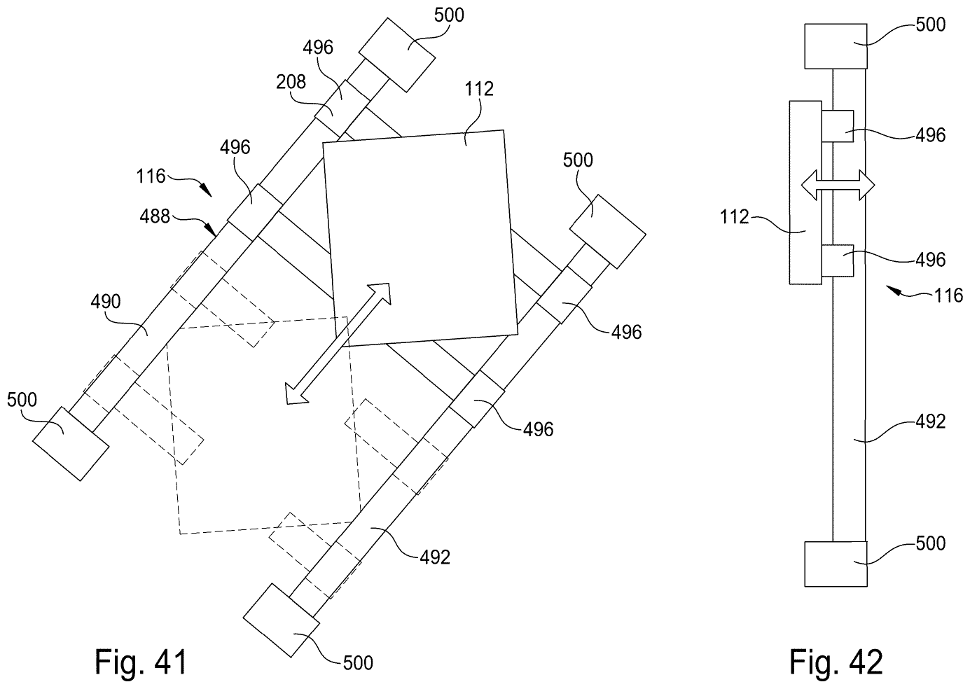

Referring to FIGS. 41 and 42, shown there is a part of a transfer device 100 according to a further exemplary embodiment, in which the opener apparatus 116 is designed differently in comparison to the preceding figures.

The opener apparatus 116 illustrated in FIGS. 41 and 42 is formed substantially like the opener apparatus 116 illustrated in FIGS. 37 and 38, so that only the differences are described below.

In comparison to the opener apparatus 116 illustrated in FIGS. 37 and 38, for example, the opener apparatus 116 can be disposed obliquely, for example so as to be rotated by 45°.

It is understood that the respective movements which initiate the second door element 112 to open and/or close the access opening 124 are correspondingly oriented differently.

The opener apparatus 116 has a plurality of, presently four, eccentric apparatuses 500, by way of which the guide elements 490, 492 are fixed on the wall portion 126.

In particular, an eccentric apparatus 500 is in each case disposed on a respective end region of a respective guide element 490, 492.

The eccentric apparatuses 500 are able to be activated by means of the motor apparatus 208 in order to move the guide elements 490, 492 away from the wall portion 126 and back toward the latter again.

The second door element 112 is thus linearly movable out of the access opening 124 and/or back into the latter by means of the eccentric apparatus 500.

In other words, the second door element 112 is movable conjointly with the guide elements 490, 492 linearly away from the access opening 124 and/or toward the access opening 124 by means of the eccentric apparatus 500.

Furthermore, by means of the motor apparatus 208, the second door element 112, in a state in which it has been moved out of the access opening 124, is able to be moved obliquely away from the access opening 124 and/or back to the latter by way of the guide elements 490, 492 (cf. arrow in FIG. 41).

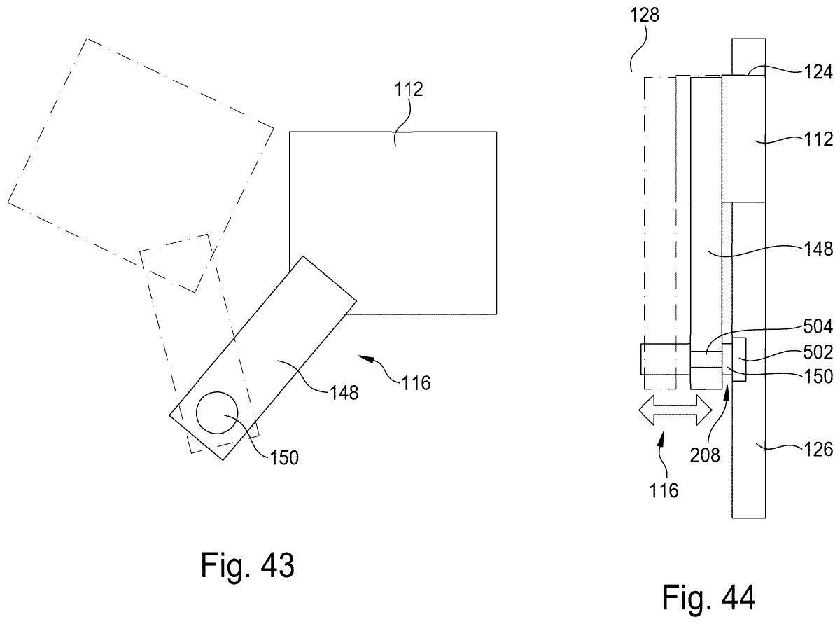

Referring to FIGS. 43 and 44, shown there is a part of a transfer device 100 according to a further exemplary embodiment, in which the opener apparatus 116 is designed differently in comparison to the preceding figures.

The opener apparatus 116 illustrated in FIGS. 43 and 44 enables substantially a motion sequence such as with the opener apparatus 116 illustrated in FIGS. 19 to 33.

A first and a second motor 502, 504 are disposed between the second process unit 104, in particular the wall portion 126, and the lever element 148.

The second motor 504 is disposed between the first motor 502 and the lever element 148.

The first motor 502 is disposed between the second motor 504 and the second process unit 104, in particular the wall portion 126.

Preferably, the first and the second motor 502, 504 are connected to one another by way of the center of rotation 150.

The first and the second motor can, for example, form a motor apparatus 208 and/or be comprised by the latter.

The first motor 502 is a motor for initiating a linear movement, e.g. a spindle drive motor, and the second motor 504 is a motor for initiating a rotating movement, e.g. a rotational motor.

The first motor 502 can move the second door element 112, or the mutually engaged door elements 110, 112 conjointly, linearly in the direction of the interior 128 of the second process unit 104 and out of the access opening 124 and also back again (cf. arrow in FIG. 44).

Subsequently, the second motor 504 can pivot the second door element 112, or the mutually engaged door elements 110, 112 conjointly, in the interior 128 and preferably away from the access opening 124 (e.g. to the dashed state in FIG. 43), preferably for an opening procedure.

A closing procedure can take place in the reverse order.

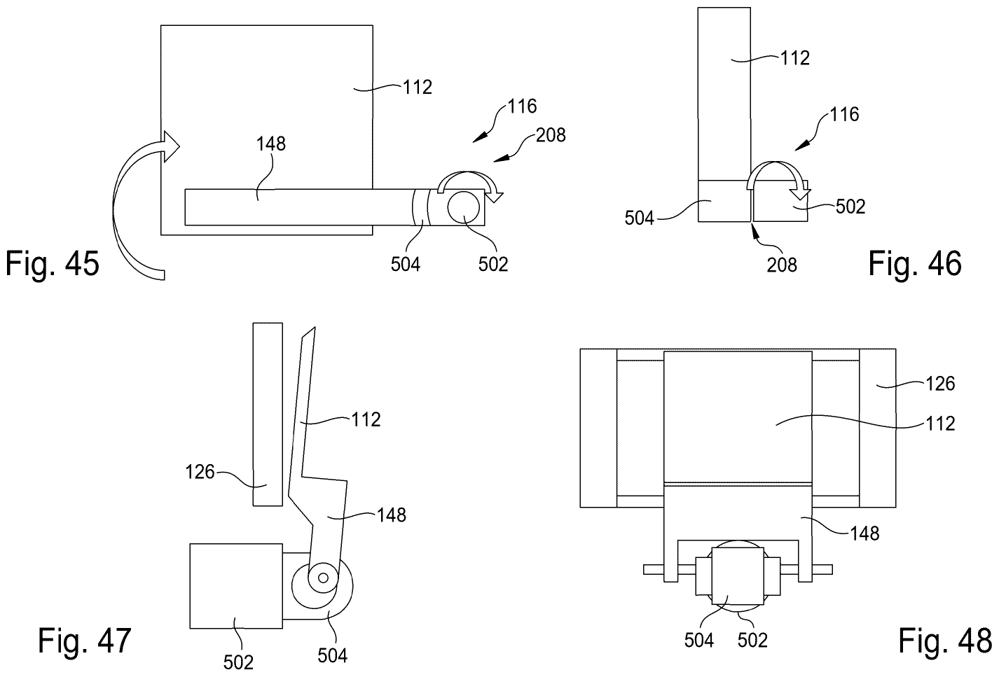

Referring to FIGS. 45 and 46, shown there is a part of a transfer device 100 according to a further exemplary embodiment, in which the opener apparatus 116 is designed differently in comparison to the preceding figures.

The opener apparatus 116 illustrated in FIGS. 45 and 46 is formed substantially like the opener apparatus 116 illustrated in FIGS. 43 and 44, so that only the differences are described below.

A first and a second motor 502, 504 are disposed between the second process unit 104, in particular the wall portion 126, and the lever element 148.

The second motor 504 is disposed between the first motor 502 and the lever element 148.

The first motor 502 is disposed between the second motor 504 and the second process unit 104, in particular the wall portion 126.

The first and the second motor can, for example, form a motor apparatus 208 and/or be comprised by the latter.

The first and second motor 502, 504 are each a motor for initiating a rotating movement, e.g. a rotational motor.

The first and the second motor 502, 504 are disposed transversely and in particular perpendicularly to one another; in other words, their respective centers of rotation are disposed transversely and in particular perpendicularly to one another.

The second motor 504 can pivot the second door element 112, or the mutually engaged door elements 110, 112 conjointly, in the direction of the interior 128 of the second process unit 104 and out of the access opening 124, and also back again (see left arrow in FIG. 45).

Subsequently, the first motor 502 can pivot the second door element 112, or the mutually engaged door elements 110, 112 conjointly, in the interior 128 and preferably away from the access opening 124 (e.g. right arrow in FIG. 45 and arrow in FIG. 46), preferably for an opening procedure.

A closing procedure can take place in the reverse order.

Referring to FIGS. 47 and 48, shown there is a part of a transfer device 100 according to a further exemplary embodiment, in which the opener apparatus 116 is designed differently in comparison to the preceding figures.

The opener apparatus 116 illustrated in FIGS. 47 and 48 is formed substantially like the opener apparatus 116 illustrated in FIGS. 45 and 46, so that only the differences are described below.

The first and the second motor 502, 504, designed as a rotational motor, are fastened directly to one another.

As can be seen from the respective figures, the lever element 148 is disposed eccentrically relative to a, or the, center of rotation of the second motor 504.

Thus, for example, a larger movement path by means of the rotation of the second motor 504 than in the opener apparatus 116 shown in FIGS. 45 and 46 can be enabled, as a result of which, for example, wear on the sealing apparatus 134, or the sealing sub-elements thereof, is able to be reduced and or avoided.

Additionally, for example, the larger movement path can allow greater construction tolerances of the components and/or parts used.

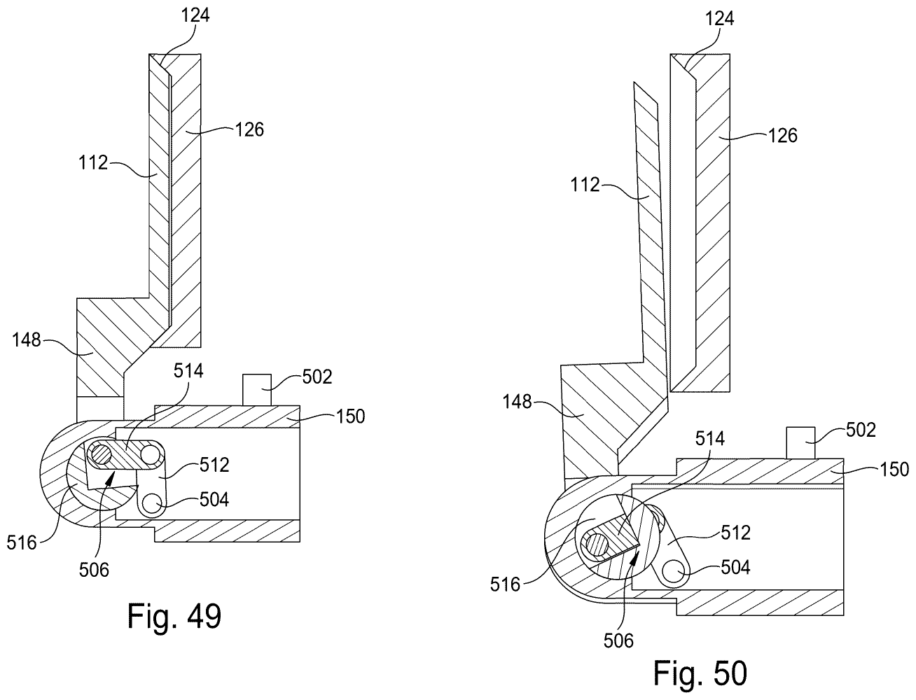

Referring to FIGS. 49 and 50, shown there is a part of a transfer device 100 according to a further exemplary embodiment, in which the opener apparatus 116 is designed differently in comparison to the preceding figures.

The opener apparatus 116 illustrated in FIGS. 49 and 50 is formed substantially like the opener apparatus 116 illustrated in FIGS. 47 and 48, so that only the differences are described below.

The lever element 148 is connected to the second motor 504 by way of a guide kinematics 506, for example a parallelogram kinematics, so that a rotating movement which is able to be initiated, or is initiated, by the second motor 504 makes it possible to initiate a circular movement of the lever element 148, and thus of the second door element 112 or the mutually engaged door elements 110, 112, away from the access opening 124.

For example, the kinematics 506 has at least two movably connected kinematics links 512, 514 and a drive disk 516 to which the lever element 148 is indirectly or directly connected. For example, the drive disk 516 can be part of a shaft and/or center of rotation, on which the lever element 148 can be held eccentrically.

The second motor 504 is connected to a first kinematics link 512 for initiating a pivoting movement of the latter, preferably a pivoting movement along a direction oriented transversely and preferably perpendicularly to the access opening 124.

The first kinematics link 512 is movably connected to the second kinematics link 514, which (second kinematics link 514) is fixed eccentrically and movably on the drive disk 516.

As a result, owing to the fact that the first kinematics link 512 is operatively connected to the drive disk 516 by way of the second kinematics link 514, a pivoting movement of the first kinematics link 512 can initiate a rotation of the drive disk 516, whereby ultimately the second door element 112, or the mutually engaged door elements 110, 112, is/are able to be moved from the access opening 124 or back into the latter.

Preferably, the second door element 112 or the mutually engaged door elements 110, 112 can remain aligned so as to be substantially parallel to the access opening 124 during this circular movement.

In other words, in order to avoid an angle between the second door element 112 and the access opening 124 in the exemplary embodiment of FIGS. 47 and 48, a guide kinematics 506, for example a parallelogram kinematics, is provided and used within the center of rotation 150, which causes the second door element 112 to move on a circular path without an opening angle. This allows the subsequent rotation by the first motor 502 to take place with a small space requirement.

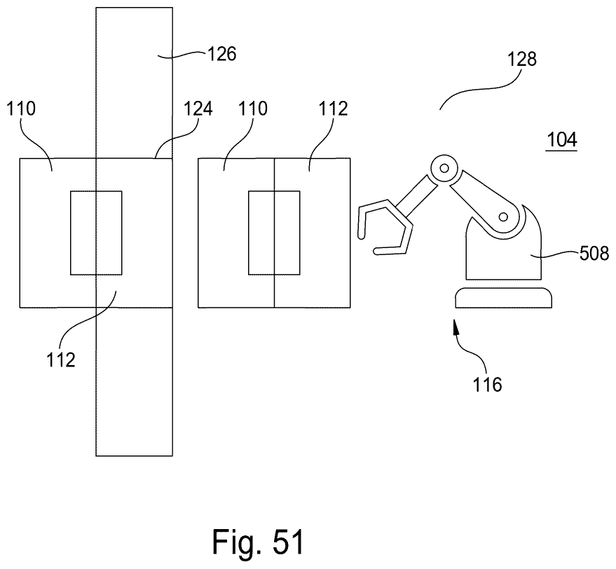

Referring to FIG. 51, shown there is a part of a transfer device 100 according to a further exemplary embodiment, in which the opener apparatus 116 is formed differently in comparison to the preceding figures.

The opener apparatus 116 is presently formed by and/or comprises a handling apparatus 508

The handling apparatus 508 is designed, for example, as a multi-axis robot arm and/or pick-and-place robot.

The handling apparatus 508 in the installed state is disposed in the second process unit 104 and is optionally rail-guided and/or displaceable for a wide and in particular larger effective range and in particular handling range.

By means of the handling apparatus 508, a respective second door element 112, or the mutually engaged door elements 110, 112 conjointly, is/are able to be moved into the interior 128 of the second process unit 104 and thus removed from respective access openings 118, 124, as is schematically illustrated in the figure.

In other words, by means of the handling apparatus 508, the second door element 112, or the mutually engaged door elements 110, 112 conjointly, is/are able to be moved, preferably in the manner of a plug, out of the access openings 118, 124 and moved back into the latter, preferably able to be moved into the interior 128 of the second process unit 104 and thus removed from the access openings 118, 124.

Further, the second door element 112 or the mutually engaged door elements 110, 112 conjointly can also be able to be moved in this way back into the access openings 118, 124.

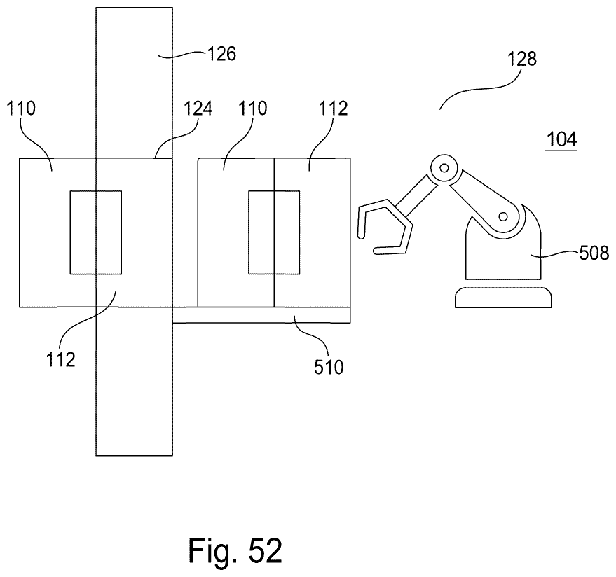

Referring to FIG. 52, shown there is a part of a transfer device 100 according to a further exemplary embodiment, in which the opener apparatus 116 is formed differently in comparison to the preceding figures.

The opener apparatus 116 illustrated in FIG. 52 is formed substantially like the opener apparatus 116 illustrated in FIG. 51, so that only the differences are described in the following.

In the present exemplary embodiment, a pull-out guide 510, e.g. telescopic guide, is disposed on the access opening 124 of the second process unit 104.

The second door element 112, or the mutually engaged door elements 110, 112 conjointly, is/are able to be moved, preferably in the manner of a drawer, along the pull-out guide 510 out of the access openings 118, 124 and back into the latter by means of the handling apparatus 508, in particular able to be moved into the interior 128 of the second process unit 104 and thus removed from the access openings 118, 124.

Further, the second door element 112 or the mutually engaged door elements 110, 112 conjointly can also be able to be moved in this way back into the access openings 118, 124.

For example, the second door element 112 can be able to be coupled, and/or can be coupled, to a displacement device, e.g. a slide and/or carriage, of the pull-out guide.

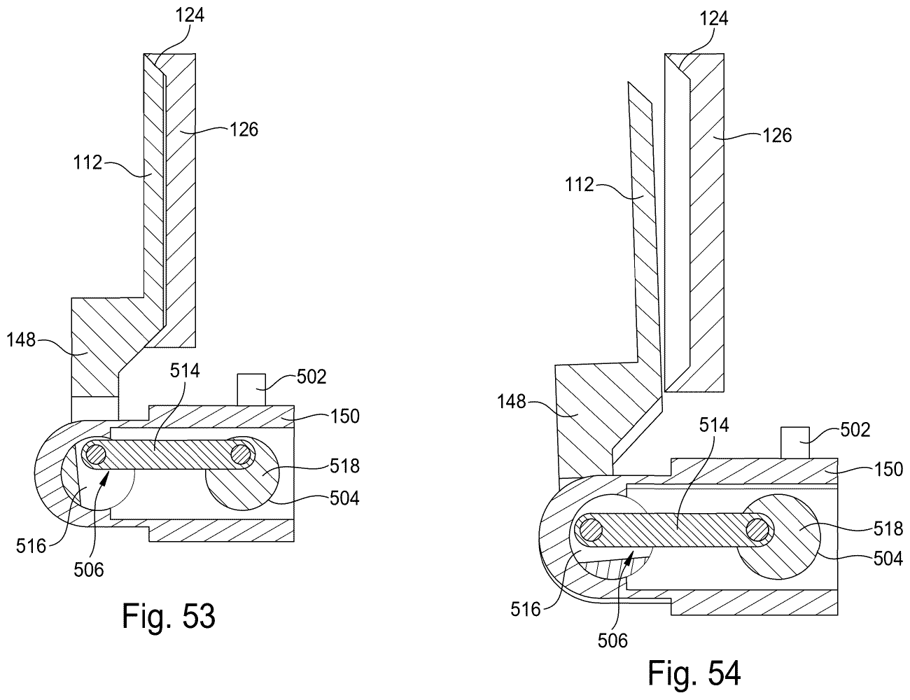

Referring to FIGS. 53 and 54, shown there is a part of a transfer device 100 according to a further exemplary embodiment, in which the opener apparatus 116 is designed differently in comparison to the preceding figures.

The opener apparatus 116 illustrated in FIGS. 53 and 54 is formed substantially like the opener apparatus 116 illustrated in FIGS. 49 and 50, so that only the differences are described below.

As can be seen in FIGS. 53 and 54, the kinematics 506 has at least one kinematics link 514, here the second kinematics link 514, the drive disk 516 and a further drive disk 518.

The further drive disk 518 is fixed on the second motor 504, for transmitting a torque and/or a rotating movement.

The kinematics link 514 is disposed eccentrically and movably on the drive disk 516 as well as on the further drive disk 518.

In other words, in comparison to FIGS. 49 and 50, the further drive disk 518 replaces the first kinematics link 512 in this exemplary embodiment.

A rotation of the further drive disk 518 can initiate the kinematics link 514 to perform a two-dimensional movement in a plane oriented transversely and in particular perpendicularly to the center of rotation of the further drive disk 518, wherein the drive disk 516 is able to be initiated to rotate by means of this movement of the kinematics link 514.

The kinematics link 514 thus enables an operative connection between the two drive disks 516, 518.

As a result, owing to the fact that the drive disks 516, 518 are operatively connected to one another by way of the kinematics link 514, a rotating movement of the further drive disk 518 (initiated by the second motor 504) can initiate a rotation of the drive disk 516, whereby ultimately the second door element 112, or the mutually engaged door elements 110, 112, is/are able to be moved from the access opening 124 or moved back into the latter.

### LIST OF REFERENCE SIGNS

100 Transfer device
102 First object, process unit, first process unit
104 Second object, further process unit, second process unit
106 First part of transfer device
108 Second part of transfer device
110 First door element
112 Second door element
114 Locking apparatus
116 Opener apparatus
118 Access opening of first process unit
120 Wall portion of first process unit
122 Interior of first process unit
124 Access opening of second process unit
126 Wall portion of second process unit
128 Interior of second process unit
130 Truncated conical portion of first door element
132 Truncated conical portion of second door element
134 Sealing apparatus
136 First sealing sub-element
138 Second sealing sub-element
140 Third sealing sub-element
142 Fourth sealing sub-element
144 Coupling element
146 Control unit
148 Lever element
150 Center of rotation
152 First end region of lever element
154 Second end region of lever element
156 First sensor unit
158 Second sensor unit
160 Third sensor unit
162 Holding portion
164 Holding contour
166 First holding element
168 Second holding element
170 Drive apparatus
172 Fourth sensor unit
174 Spring element
176 First shaft
178 Second shaft
180 Motor
182 Spindle drive
184 Activating arm
200 System
202 Third process unit
204 Fourth process unit
206 Separate interior space
208 Motor apparatus
210 Energy store
300 Eccentric apparatus
302 Shaft
304 Motion apparatus of eccentric apparatus
306 Eccentric element
308 Counter-element
310 Wedge apparatus
312 Displacement element
314 Motion apparatus of wedge apparatus
316 Wedge-shaped element
318 Wedge-shaped counter-element
320 Tapered portion
322 First lever link
324 Second lever link
326 Articulation apparatus
328 Gripper actuator mechanism
330 Further locking apparatus
332 Bayonet element
334 Bayonet counter-element
336 Clamping and/or tensioning lever
338 Spring element of bell-crank lever apparatus
340 Shaft
342 Eccentric element
400 Magnetic element of locking apparatus
402 Receptacle bore
404 Magnetic element of further locking apparatus
406 Holding component of further locking apparatus
408 Receptacle bore of holding component
410 Counter-element
412 Solenoid of coupling element
414 Receptacle pocket
416 Cover element
418 Drive unit
420 Motion apparatus
422 Motor apparatus
424 Displacement device
426 Cylindrical portion
428 Cuboid portion
430 Fastening portion
432 Bearing device
434 Further sealing apparatus
436 Carriage unit
438 Carriage portion
440 Connecting portion
442 Roller element
444 Rotational motor
446 Spindle drive motor
448 Spindle
450 Partition wall
452 Spindle bearing device
454 Lid element
456 Displacement element
458 Linear guide
460 Entrainment magnet
462 Entrainment counter-element
464 Braking apparatus
466 Magazine device
468 Positioning element
470 Complementary positioning element
472 Plug element
474 Socket element
476 Fifth sensor unit
478 Locking element
480 Third lever link
482 Parallelogram guide
484 Motor
486 Lever kinematics
488 Guide apparatus
490 First guide element
492 Second guide element
494 Holding kinematics
496 Holding link
498 Laminar flow
500 Eccentric apparatus
502 First motor
504 Second motor
506 Guide kinematics
508 Handling apparatus
510 Pull-out guide
512 First kinematics link
514 Second kinematics link
516 Drive disk
518 Further drive disk
A Opening rotation direction
B Closing rotation direction

1. A transfer device for enabling an optionally aseptic transition between a first object or a first process unit, and a second object or a second process unit, wherein the transfer device is capable of being installed at least partially in the first object and at least partially in the second object, the transfer device comprising:
a first door element, which in the installed state is disposed on the first object, for opening and closing an access opening formed in a wall portion of the first object and leading to an interior of the first object;
a second door element, which in the installed state is disposed on the second object, for opening and closing an access opening formed in a wall portion of the second object and leading to an interior of the second object;
a locking apparatus for locking the first door element in a closed state and for releasing the first door element for opening the first door element, wherein the locking apparatus in the installed state is disposed in and/or on the first object; and
an opener apparatus for opening and/or closing at least one of the door elements, wherein the opener apparatus in the installed state is disposed in and/or on the second object,
wherein the first door element and the second door element are able to be brought to engage with one another and in the engaged state, when the first door element is released by means of the locking apparatus, are able to be conjointly removed from the access openings by means of the opener apparatus, so as to enable the transition, or the aseptic transition, between the first object and the second object.

2. The transfer device as claimed in claim 1,
wherein the first door element comprises a portion, or an truncated conical or truncated pyramidal portion, which tapers in a direction which, in the closed state of the first door element, is directed toward the interior of the first object, wherein a circumferential surface of said portion of the first door element, in the closed state of the first door element, rests on an inner circumferential surface of the access opening of the first object, and/or
the second door element comprises a portion, or an truncated conical or pyramidal portion, which tapers in a direction which, in the closed state of the second door element, is directed outward relative to the second object, wherein a circumferential surface of said portion of the second door element, in the closed state of the second door element, rests on an inner circumferential surface of the access opening of the second object.

3. The transfer device as claimed in claim 1,
wherein the transfer device has a sealing apparatus which is formed from or comprises a plurality of sealing sub-elements, wherein at least one of the following applies:
in the closed state of the first door element, the first door element and an inner circumferential surface of the access opening of the first object are able to be sealed, or are sealed, in relation to the outside by means of the sealing apparatus, and/or
in the closed state of the second door element, the second door element and an inner circumferential surface of the access opening of the second object are able to be sealed, or are sealed, in relation to the outside by means of the sealing apparatus, and/or
in the engaged state of the first and the second door element, respective surfaces of both door elements, which in the respective closed state of the corresponding door elements are directed outward relative to the corresponding object, are able to be sealed, or are sealed, in relation to the outside by means of the sealing apparatus, and/or
in a state in which the respective wall portions of the objects rest on one another for bringing the first and the second door element to engage, respective opening regions of both access openings of both objects are able to be sealed, or are sealed, in relation to the outside by means of the sealing apparatus, and/or
the sealing apparatus and/or each sealing sub-element has a closed and/or circular contour.

4. The transfer device as claimed in claim 1,
wherein the second door element comprises a coupling element which is provided in and/or on the second door element and forms at least part of the second door element, wherein the first door element is able to be brought to engage with the second door element and able to be held thereon by means of the coupling element, and/or wherein at least one of the following is provided:
the coupling element forms a part of the second door element which is directed outward relative to the second object and has a flush transition to the remaining part of the second door element that is directed outward relative to the second object, and/or
the coupling element is designed as a solenoid or comprises at least one solenoid, and the first door element is partially or completely formed as or comprises a magnetic counter-element.

5. The transfer device as claimed in claim 1,
wherein the first door element and/or the second door element each have an outwardly directed shell surface with a hygienically compliant design or a hygienic design, and/or the first door element and/or the second door element have no undercuts on the outside and/or on the shell surface.

6. The transfer device as claimed in claim 1,
wherein the opener apparatus comprises a pivotable lever element which, in the installed state in the second object, is pivotably fixed on a first end region of the lever element, and on which the second door element is fastened to a second end region of the lever element, wherein the second door element, or the mutually engaged door elements conjointly, is/are able to be moved into the interior of the second object and thus able to be removed from the access openings by means of the lever element.

7. The transfer device as claimed in claim 1,
wherein at least a part of the opener apparatus is rotatable and/or pivotable about a center of rotation which is able to be disposed, or is disposed, in the interior of the second object, wherein the opening and closing of at least one of the door elements is able to be initiated by means of a corresponding rotating and/or pivoting movement, wherein the center of rotation in the installed state, relative to the access opening of the second object, is disposed at a spacing which is greater than, or greater than or equal to, a widest opening dimension of the access opening of the second object, or than an opening diameter of the access opening of the second object.

8. The transfer device as claimed in claim 1,
wherein the transfer device comprises one or a plurality of sensor units for detecting an open state of the first door element and/or for detecting an open state of the second door element, and/or wherein each sensor unit is connected, for signaling, to a control unit of the transfer device and/or to a control unit which is assignable, or is assigned, to the transfer device, for determining whether a transition between the first and the second object is enabled.

9. The transfer device as claimed in claim 1,
wherein the transfer device comprises at least one sensor unit by means of which a state is detectable whether the first door element and the second door element are engaged with one another, and/or wherein at least one of the following is provided:
a mass moved by the opener apparatus during opening and/or closing is detectable by means of the sensor unit, and/or
a change in a magnetic field in and/or on the second object is detectable by means of the sensor unit, which change is able to be caused by the opener apparatus when opening and/or closing, and/or
an electrical contact, which is closed in the engaged state of the first and the second door element, is detectable by means of the sensor unit.

10. The transfer device as claimed in claim 1,
wherein the first door element comprises a holding contour which, in the closed state, is disposed internally relative to the first object, wherein the locking apparatus comprises:
at least one holding element which, in the installed state, is disposed in the first object and which is selectively able to be brought to engage with the holding contour so as to lock the first door element, and
a drive apparatus by means of which the holding element is movable in order to selectively bring the holding element to engage with the holding contour, and/or is movable between a locked position and a release position, and/or wherein the drive apparatus in the installed state is disposed in and/or on the first object, and/or wherein the drive apparatus comprises an elastic element, or a spring element, by means of which the holding element is able to be preloaded, or is preloaded, in the direction of engagement with the holding contour and/or in the direction of the locked position, and/or
wherein the transfer device and/or the locking apparatus comprise at least one sensor unit for detecting a locked state of the first door element, by means of which a position of the holding element relative to the drive apparatus and/or the holding contour is detectable.

11. The transfer device as claimed in claim 10,
wherein the drive apparatus is operatively connected to the holding element by way of a shaft, and by way of which shaft the holding element is able to be brought to engage with the holding contour and brought back out of the engagement, and/or is movable into the locked position and the release position, and/or wherein one of the following applies:
the drive apparatus comprises a motor and a positive-ratio gearbox by way of which a torque which is able to be initiated by the motor and/or a force which is able to be initiated by the motor is able to be delivered, and/or delivered to the shaft, for bringing the holding element to engage with the holding contour, and/or for moving the holding element into the locked position and the release position; and/or
the drive apparatus comprises a motor and a spindle drive driven by means of the motor, and formed on the shaft is an activating arm, which is movably connected to the spindle drive, wherein a rotating movement of the spindle drive is able to be transmitted to the shaft by way of the connection of the shaft to the activating arm in the form of a pivoting movement which is able to be transmitted to the holding element by way of the shaft, and/or wherein the locking apparatus comprises a further holding element which is movably connected to the spindle drive by way of a further shaft, by means of an activating arm formed on the further shaft, wherein the two holding elements are able to be brought to engage with the holding contour and brought back out of the engagement, and/or are movable into the locked position and the release position, by means of a mutually opposing and/or synchronous pivoting movement.

12. The transfer device as claimed in claim 10,
wherein the drive apparatus in the installed state is disposed on the first object and outside the interior of the first object, or
the drive apparatus in the installed state is disposed within the first object and is designed to be at least partially autoclavable, wherein an autoclavable part of the drive apparatus and a non-autoclavable part of the drive apparatus are able to be connected, or are connected, to one another by way of at least one coupling component, and the non-autoclavable part of the drive apparatus can be releasably installed in the interior of the first object, or
the drive apparatus in the installed state is disposed within the first object and is designed to be autoclavable, and/or wherein the drive apparatus comprises a housing, or a sealed housing, which is partially or completely, or thermally, insulated relative to an environment of the housing.

13. The transfer device as claimed in claim 1,
wherein the opener apparatus has a drive unit by means of which the second door element, or the mutually engaged door elements conjointly, is/are movable in a pivoting and/or linear manner, or in a linear and then pivoting manner, into the interior of the second object, and/or whereby these are removable out of and/or from the access openings.

14. The transfer device as claimed in claim 13,
wherein the drive unit has a motion apparatus and a motor apparatus, wherein the motion apparatus is rotatable and at least partially linearly movable by means of the motor apparatus, and/or
wherein the motion apparatus has a center of rotation, which is formed as a shaft, and a displacement device, wherein the displacement device is disposed externally on the center of rotation and is movable and/or displaceable linearly along the center of rotation, and/or
wherein the motor apparatus has a rotational motor for initiating a rotating movement of the motion apparatus, and a linear motor, or a linear motor being designed as a spindle drive motor, for initiating a linear movement of at least one part, or the displacement device, of the motion apparatus.

15. The transfer device as claimed in claim 1,
wherein the transfer device comprises a control unit, and/or the transfer device is assignable, or is assigned, a control unit, or a control unit of the first and/or of the second object, wherein the control unit and/or its signaling connections capable of being disconnected and/or de-energized for autoclaving the transfer device.

16. A system consisting of at least two objects, or process units, wherein the two objects comprise at least one transfer device as claimed in claim 1, wherein each object serves to manufacture a product or a biological-pharmaceutical product.

17. A system consisting of three or four or more objects or process units, wherein provided in each object is one or a plurality of first and/or one or a plurality of second parts of a transfer device as claimed in claim 1, wherein the transfer device capable of being formed from at least one first and at least one second part, and wherein at least one first part and at least one second part of the transfer device are provided in mutually different objects.
